SEQUENCE LISTING (60 sequences)

SEQ ID NO: 1 - DNA, 72 bp, Homo sapiens
aagatactgataattccagtttgtcaccaccacctgatgttactttaagcttactcccttcaaacgaaacag

SEQ ID NO: 2 - DNA, 114 bp, Homo sapiens
aagatactgataattccagtttgtcaccaccacctgatgttactttaagcttactcccttcaaacgaaacagaaaaaactaaaatcactatagtaaaaaccttcaatgcttcag

SEQ ID NO: 3 - DNA, 144 bp, Homo sapiens
aagatactgataattccagtttgtcaccaccacctgctaaattatctgttgtcagttttgccccctcctccaatggtactccagaggttgaaacaacaagcctcaatgatgttactttaagcttactcccttcaaacgaaacag

SEQ ID NO: 4 - DNA, 138 bp, Homo sapiens
aagatactgataattccagtttgtcaccaccacctgaggttgaaacaacaagcctcaatgatgttactttaagcttactcccttcaaacgaaacagaaaaaactaaaatcactatagtaaaaaccttcaatgcttcag

SEQ ID NO: 5 - DNA, 96 bp, Homo sapiens
aagatactgataattccagtttgtcaccaccacctgaggttgaaacaacaagcctcaatgatgttactttaagcttactcccttcaaacgaaacag

SEQ ID NO: 6 - DNA, 118 bp, Homo sapiens
aagatactgataattccagtttgtcaccaccacctgctaaattatctgttgtcagttttgccccctcctccaatggtactccagatgttactttaagcttactcccttcaaacgaaac

SEQ ID NO: 7 - DNA, 186 bp, Homo sapiens
aagatactgataattccagtttgtcaccaccacctgctaaattatctgttgtcagttttgccccctcctccaatggtactccagaggttgaaacaacaagcctcaatgatgttactttaagcttactcccttcaaacgaaacagaaaaaactaaaatcactatagtaaaaaccttcaatgcttcag

SEQ ID NO: 8 - DNA, 91 bp, Homo sapiens
cgggagcggcgggggcggcgccgaggccgcgggcggggggaggagggccgcagccccggagaggggaaaaggcgggccggacggcgccagc

SEQ ID NO: 9 - DNA, 162 bp, Mus musculus
aaaacggtaattccagtttgttatcaccatctgctgaatcatctcttgtcagtctcatcccctactccaatggtacaccagatgctgcttcagaagttttgtcgactttaaacaaaacagaaaaatctaaaatcactatagtaaaaaccttcaatgcatcag

SEQ ID NO: 10 - DNA, 114 bp, Mus musculus
aaaacggtaattccagtttgttatcaccatctgatgctgcttcagaagttttgtcgactttaaacaaaacagaaaaatctaaaatcactatagtaaaaaccttcaatgcatcag

SEQ ID NO: 11 - DNA, 153 bp, Mus musculus
aaaacggtaattccagtttgttatcaccatctgctgaatcatctcttgtcagtctcatcccctactccaatgatgctgcttcagaagttttgtcgactttaaacaaaacagaaaaatctaaaatcactatagtaaaaaccttcaatgcatcag

SEQ ID NO: 12 - DNA, 81 bp, Mus musculus
atgctgcttcagaagttttgtcgactttaaacaaaacagaaaaatctaaaatcactatagtaaaaaccttcaatgcatcag

SEQ ID NO: 13 - DNA, 160 bp, Rattus norvegicus
aaaatgctggtaattccagtttgttgtcaccatctgctgaatcgtctcttgtcagtcttgttccctactccaatggtacaccagatgctgcttccgaagtattgtcgactttaaacagaacagaaaaatctaaaatcactatattaaaaaccttcaatgc

SEQ ID NO: 14 - DNA, 117 bp, Rattus norvegicus
aaaatgctggtaattccagtttgttgtcaccatctgatgctgcttccgaagtattgtcgactttaaacagaacagaaaaatctaaaatcactatattaaaaaccttcaatgcatcag

SEQ ID NO: 15 - DNA, 81 bp, Rattus norvegicus
atgctgcttccgaagtattgtcgactttaaacagaacagaaaaatctaaaatcactatattaaaaaccttcaatgcatcag

SEQ ID NO: 16 - DNA, 4651 bp, Homo sapiens
cgggagcggcgggggcggcgccgaggccgcgggcggggggaggagggccgcagccccggagaggggaaaaggcgggccggacggcgccagcagccagcccgaggacgcgagcggcaggtgtgcacagaggttctccactttgttttctgaactcgcggtcaggatggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgatgttactttaagcttactcccttcaaacgaaacaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgcaatagccgctttggaaagagtaaagattcgaccaatggaacactgctgctgttctgtcaggataccctgcccttcctccccagaagagttgggaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtgattcctttcttctaaaatcaaagcatgatgcttgacagtgtgaaatgtccaattttaccttttacacaatgtgagatgtatgaaaatcaactcattttattctcggcaacatctggagaagcataagctaattaagggcgatgattattattacaagaagaaaccaagacattacaccatggtttttagacatttctgatttggtttcttatctttcattttataagaaggttggttttaaacaatacactaagaatgactcctataaagaaaacaaaaaaaggtagtgaactttcagctaccttttaaagaggctaagttatctttgataacatcatataaagcaactgttgacttcagcctgttggtgagtttagttgtgcatgcctttgttgtatataagctaaattctagtgacccatgtgtcaaaaatcttacttctacatttttttgtatttattttctactgtgtaaatgtattcctttgtagaatcatggttgttttgtctcacgtgataattcagaaaatccttgctcgttccgcaaatcctaaagctccttttggagatgatataggatgtgaaatacagaaacctcagtgaaatcaagaaataatgatcccagccagactgagaaaatgtaagcagacagtgccacagttagctcatacagtgcctttgagcaagttaggaaaagatgcccccactgggcagacacagccctatgggtcatggtttgacaaacagagtgagagaccatattttagccccactcaccctcttgggtgcacgacctgtacagccaaacacagcatccaatatgaatacccatcccctgaccgcatccccagtagtcagattatagaatctgcaccaagatgtttagctttataccttggccacagagagggatgaactgtcatccagaccatgtgtcaggaaaattgtgaacgtagatgaggtacatacactgccgcttctcaaatccccagagcctttaggaacaggagagtagactaggattccttctcttaaaaaggtacatatatatggaaaaaaatcatattgccgttctttaaaaggcaactgcatggtacattgttgattgttatgactggtacactctggcccagccagagctataattgttttttaaatgtgtcttgaagaatgcacagtgacaaggggagtagctattgggaacagggaactgtcctacactgctattgttgctacatgtatcgagccttgattgctcctagttatatacagggtctatcttgcttcctacctacatctgcttgagcagtgcctcaagtacatccttattaggaacatttcaaaccccttttagttaagtctttcactaaggttctcttgcatatatttcaagtgaatgttggatctcagactaaccatagtaataatacacatttctgtgagtgctgacttgtctttgcaatatttcttttctgatttatttaattttcttgtatttatatgttaaaatcaaaaatgttaaaatcaatgaaataaatttgcagttaaga

SEQ ID NO: 17 - DNA, 4693 bp, Homo sapiens
cgggagcggcgggggcggcgccgaggccgcgggcggggggaggagggccgcagccccggagaggggaaaaggcgggccggacggcgccagcagccagcccgaggacgcgagcggcaggtgtgcacagaggttctccactttgttttctgaactcgcggtcaggatggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgatgttactttaagcttactcccttcaaacgaaacagaaaaaactaaaatcactatagtaaaaaccttcaatgcttcaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgcaatagccgctttggaaagagtaaagattcgaccaatggaacactgctgctgttctgtcaggataccctgcccttcctccccagaagagttgggaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtgattcctttcttctaaaatcaaagcatgatgcttgacagtgtgaaatgtccaattttaccttttacacaatgtgagatgtatgaaaatcaactcattttattctcggcaacatctggagaagcataagctaattaagggcgatgattattattacaagaagaaaccaagacattacaccatggtttttagacatttctgatttggtttcttatctttcattttataagaaggttggttttaaacaatacactaagaatgactcctataaagaaaacaaaaaaaggtagtgaactttcagctaccttttaaagaggctaagttatctttgataacatcatataaagcaactgttgacttcagcctgttggtgagtttagttgtgcatgcctttgttgtatataagctaaattctagtgacccatgtgtcaaaaatcttacttctacatttttttgtatttattttctactgtgtaaatgtattcctttgtagaatcatggttgttttgtctcacgtgataattcagaaaatccttgctcgttccgcaaatcctaaagctccttttggagatgatataggatgtgaaatacagaaacctcagtgaaatcaagaaataatgatcccagccagactgagaaaatgtaagcagacagtgccacagttagctcatacagtgcctttgagcaagttaggaaaagatgcccccactgggcagacacagccctatgggtcatggtttgacaaacagagtgagagaccatattttagccccactcaccctcttgggtgcacgacctgtacagccaaacacagcatccaatatgaatacccatcccctgaccgcatccccagtagtcagattatagaatctgcaccaagatgtttagctttataccttggccacagagagggatgaactgtcatccagaccatgtgtcaggaaaattgtgaacgtagatgaggtacatacactgccgcttctcaaatccccagagcctttaggaacaggagagtagactaggattccttctcttaaaaaggtacatatatatggaaaaaaatcatattgccgttctttaaaaggcaactgcatggtacattgttgattgttatgactggtacactctggcccagccagagctataattgttttttaaatgtgtcttgaagaatgcacagtgacaaggggagtagctattgggaacagggaactgtcctacactgctattgttgctacatgtatcgagccttgattgctcctagttatatacagggtctatcttgcttcctacctacatctgcttgagcagtgcctcaagtacatccttattaggaacatttcaaaccccttttagttaagtctttcactaaggttctcttgcatatatttcaagtgaatgttggatctcagactaaccatagtaataatacacatttctgtgagtgctgacttgtctttgcaatatttcttttctgatttatttaattttcttgtatttatatgttaaaatcaaaaatgttaaaatcaatgaaataaatttgcagttaaga

SEQ ID NO: 18 - DNA, 4723 bp, Homo sapiens
cgggagcggcgggggcggcgccgaggccgcgggcggggggaggagggccgcagccccggagaggggaaaaggcgggccggacggcgccagcagccagcccgaggacgcgagcggcaggtgtgcacagaggttctccactttgttttctgaactcgcggtcaggatggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgctaaattatctgttgtcagttttgccccctcctccaatggtactccagaggttgaaacaacaagcctcaatgatgttactttaagcttactcccttcaaacgaaacaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgcaatagccgctttggaaagagtaaagattcgaccaatggaacactgctgctgttctgtcaggataccctgcccttcctccccagaagagttgggaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtgattcctttcttctaaaatcaaagcatgatgcttgacagtgtgaaatgtccaattttaccttttacacaatgtgagatgtatgaaaatcaactcattttattctcggcaacatctggagaagcataagctaattaagggcgatgattattattacaagaagaaaccaagacattacaccatggtttttagacatttctgatttggtttcttatctttcattttataagaaggttggttttaaacaatacactaagaatgactcctataaagaaaacaaaaaaaggtagtgaactttcagctaccttttaaagaggctaagttatctttgataacatcatataaagcaactgttgacttcagcctgttggtgagtttagttgtgcatgcctttgttgtatataagctaaattctagtgacccatgtgtcaaaaatcttacttctacatttttttgtatttattttctactgtgtaaatgtattcctttgtagaatcatggttgttttgtctcacgtgataattcagaaaatccttgctcgttccgcaaatcctaaagctccttttggagatgatataggatgtgaaatacagaaacctcagtgaaatcaagaaataatgatcccagccagactgagaaaatgtaagcagacagtgccacagttagctcatacagtgcctttgagcaagttaggaaaagatgcccccactgggcagacacagccctatgggtcatggtttgacaaacagagtgagagaccatattttagccccactcaccctcttgggtgcacgacctgtacagccaaacacagcatccaatatgaatacccatcccctgaccgcatccccagtagtcagattatagaatctgcaccaagatgtttagctttataccttggccacagagagggatgaactgtcatccagaccatgtgtcaggaaaattgtgaacgtagatgaggtacatacactgccgcttctcaaatccccagagcctttaggaacaggagagtagactaggattccttctcttaaaaaggtacatatatatggaaaaaaatcatattgccgttctttaaaaggcaactgcatggtacattgttgattgttatgactggtacactctggcccagccagagctataattgttttttaaatgtgtcttgaagaatgcacagtgacaaggggagtagctattgggaacagggaactgtcctacactgctattgttgctacatgtatcgagccttgattgctcctagttatatacagggtctatcttgcttcctacctacatctgcttgagcagtgcctcaagtacatccttattaggaacatttcaaaccccttttagttaagtctttcactaaggttctcttgcatatatttcaagtgaatgttggatctcagactaaccatagtaataatacacatttctgtgagtgctgacttgtctttgcaatatttcttttctgatttatttaattttcttgtatttatatgttaaaatcaaaaatgttaaaatcaatgaaataaatttgcagttaaga

SEQ ID NO: 19 - DNA, 4716 bp, Homo sapiens
cgggagcggcgggggcggcgccgaggccgcgggcggggggaggagggccgcagccccggagaggggaaaaggcgggccggacggcgccagcagccagcccgaggacgcgagcggcaggtgtgcacagaggttctccactttgttttctgaactcgcggtcaggatggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgaggttgaaacaacaagcctcaatgatgttactttaagcttactcccttcaaacgaaacagaaaaaactaaaatcactatagtaaaaaccttcaatgcttcaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgcaatagccgctttggaaagagtaaagattcgaccaatggaacactgctgctgttctgtcaggataccctgcccttcctccccagaagagttgggaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtgattcctttcttctaaaatcaaagcatgatgcttgacagtgtgaaatgtccaattttaccttttacacaatgtgagatgtatgaaaatcaactcattttattctcggcaacatctggagaagcataagctaattaagggcgatgattattattacaagaagaaaccaagacattacaccatggtttttagacatttctgatttggtttcttatctttcattttataagaaggttggttttaaacaatacactaagaatgactcctataaagaaaacaaaaaaaggtagtgaactttcagctaccttttaaagaggctaagttatctttgataacatcatataaagcaactgttgacttcagcctgttggtgagtttagttgtgcatgcctttgttgtatataagctaaattctagtgacccatgtgtcaaaaatcttacttctacatttttttgtatttattttctactgtgtaaatgtattcctttgtagaatcatggttgttttgtctcacgtgataattcagaaaatccttgctcgttccgcaaatcctaaagctccttttggagatgatataggatgtgaaatacagaaacctcagtgaaatcaagaaataatgatcccagccagactgagaaaatgtaagcagacagtgccacagttagctcatacagtgcctttgagcaagttaggaaaagatgcccccactgggcagacacagccctatgggtcatggtttgacaaacagagtgagagaccatattttagccccactcaccctcttgggtgcacgacctgtacagccaaaacagcatccaatatgaatacccatcccctgaccgcatccccagtagtcagattatagaatctgcaccaagatgtttagctttataccttggccacagagagggatgaactgtcatccagaccatgtgtcaggaaaattgtgaacgtagatgaggtacatacactgccgcttctcaaatccccagagcctttaggaacaggagagtagactaggattccttctcttaaaaaggtacatatatatggaaaaaaatcatattgccgttctttaaaaggcaactgcatggtacattgttgattgttatgactggtacactctggcccagccagagctataattgttttttaaatgtgtcttgaagaatgcacagtgacaaggggagtagctattgggaacagggaactgtcctacactgctattgttgctacatgtatcgagccttgattgctcctagttatatacagggtctatcttgcttcctacctacatctgcttgagcagtgcctcaagtacatccttattaggaacatttcaaaccccttttagttaagtctttcactaaggttctcttgcatatatttcaagtgaatgttggatctcagactaaccatagtaataatacacatttctgtgagtgctgacttgtctttgcaatatttcttttctgatttatttaattttcttgtatttatatgttaaaatcaaaaatgttaaaatcaatgaaataaatttgcagttaaga

SEQ ID NO: 20 - DNA, 4675 bp, Homo sapiens
cgggagcggcgggggcggcgccgaggccgcgggcggggggaggagggccgcagccccggagaggggaaaaggcgggccggacggcgccagcagccagcccgaggacgcgagcggcaggtgtgcacagaggttctccactttgttttctgaactcgcggtcaggatggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgaggttgaaacaacaagcctcaatgatgttactttaagcttactcccttcaaacgaaacaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgcaatagccgctttggaaagagtaaagattcgaccaatggaacactgctgctgttctgtcaggataccctgcccttcctccccagaagagttgggaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtgattcctttcttctaaaatcaaagcatgatgcttgacagtgtgaaatgtccaattttaccttttacacaatgtgagatgtatgaaaatcaactcattttattctcggcaacatctggagaagcataagctaattaagggcgatgattattattacaagaagaaaccaagacattacaccatggtttttagacatttctgatttggtttcttatctttcattttataagaaggttggttttaaacaatacactaagaatgactcctataaagaaaacaaaaaaaggtagtgaactttcagctaccttttaaagaggctaagttatctttgataacatcatataaagcaactgttgacttcagcctgttggtgagtttagttgtgcatgcctttgttgtatataagctaaattctagtgacccatgtgtcaaaaatcttacttctacatttttttgtatttattttctactgtgtaaatgtattcctttgtagaatcatggttgttttgtctcacgtgataattcagaaaatccttgctcgttccgcaaatcctaaagctccttttggagatgatataggatgtgaaatacagaaacctcagtgaaatcaagaaataatgatcccagccagactgagaaaatgtaagcagacagtgccacagttagctcatacagtgcctttgagcaagttaggaaaagatgcccccactgggcagacacagccctatgggtcatggtttgacaaacagagtgagagaccatattttagccccactcaccctcttgggtgcacgacctgtacagccaaacacagcatccaatatgaatacccatcccctgaccgcatccccagtagtcagattatagaatctgcaccaagatgtttagctttataccttggccacagagagggatgaactgtcatccagaccatgtgtcaggaaaattgtgaacgtagatgaggtacatacactgccgcttctcaaatccccagagcctttaggaacaggagagtagactaggattccttctcttaaaaaggtacatatatatggaaaaaaatcatattgccgttctttaaaaggcaactgcatggtacattgttgattgttatgactggtacactctggcccagccagagctataattgttttttaaatgtgtcttgaagaatgcacagtgacaaggggagtagctattgggaacagggaactgtcctacactgctattgttgctacatgtatcgagccttgattgctcctagttatatacagggtctatcttgcttcctacctacatctgcttgagcagtgcctcaagtacatccttattaggaacatttcaaaccccttttagttaagtctttcactaaggttctcttgcatatatttcaagtgaatgttggatctcagactaaccatagtaataatacacatttctgtgagtgctgacttgtctttgcaatatttcttttctgatttatttaattttcttgtatttatatgttaaaatcaaaaatgttaaaatcaatgaaataaatttgcagttaaga

SEQ ID NO: 21 - DNA, 4699 bp, Homo sapiens
cgggagcggcgggggcggcgccgaggccgcgggcggggggaggagggccgcagccccggagaggggaaaaggcgggccggacggcgccagcagccagcccgaggacgcgagcggcaggtgtgcacagaggttctccactttgttttctgaactcgcggtcaggatggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgctaaattatctgttgtcagttttgccccctcctccaatggtactccagatgttactttaagcttactcccttcaaacgaaacaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgcaatagccgctttggaaagagtaaagattcgaccaatggaacactgctgctgttctgtcaggataccctgcccttcctccccagaagagttgggaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtgattcctttcttctaaaatcaaagcatgatgcttgacagtgtgaaatgtccaattttaccttttacacaatgtgagatgtatgaaaatcaactcattttattctcggcaacatctggagaagcataagctaattaagggcgatgattattattacaagaagaaaccaagacattacaccatggtttttagacatttctgatttggtttcttatctttcattttataagaaggttggttttaaacaatacactaagaatgactcctataaagaaaacaaaaaaaggtagtgaactttcagctaccttttaaagaggctaagttatctttgataacatcatataaagcaactgttgacttcagcctgttggtgagtttagttgtgcatgcctttgttgtatataagctaaattctagtgacccatgtgtcaaaaatcttacttctacatttttttgtatttattttctactgtgtaaatgtattcctttgtagaatcatggttgttttgtctcacgtgataattcagaaaatccttgctcgttccgcaaatcctaaagctccttttggagatgatataggatgtgaaatacagaaacctcagtgaaatcaagaaataatgatcccagccagactgagaaaatgtaagcagacagtgccacagttagctcatacagtgcctttgagcaagttaggaaaagatgcccccactgggcagacacagccctatgggtcatggtttgacaaacagagtgagagaccatattttagccccactcaccctcttgggtgcacgacctgtacagccaaacacagcatccaatatgaatacccatcccctgaccgcatccccagtagtcagattatagaatctgcaccaagatgtttagctttataccttggccacagagagggatgaactgtcatccagaccatgtgtcaggaaaattgtgaacgtagatgaggtacatacactgccgcttctcaaatccccagagcctttaggaacaggagagtagactaggattccttctcttaaaaaggtacatatatatggaaaaaaatcatattgccgttctttaaaaggcaactgcatggtacattgttgattgttatgactggtacactctggcccagccagagctataattgttttttaaatgtgtcttgaagaatgcacagtgacaaggggagtagctattgggaacagggaactgtcctacactgctattgttgctacatgtatcgagccttgattgctcctagttatatacagggtctatcttgcttcctacctacatctgcttgagcagtgcctcaagtacatccttattaggaacatttcaaaccccttttagttaagtctttcactaaggttctcttgcatatatttcaagtgaatgttggatctcagactaaccatagtaataatacacatttctgtgagtgctgacttgtctttgcaatatttcttttctgatttatttaattttcttgtatttatatgttaaaatcaaaaatgttaaaatcaatgaaataaatttgcagttaaga

SEQ ID NO: 22 - DNA, 4765 bp, Homo sapiens
cgggagcggcgggggcggcgccgaggccgcgggcggggggaggagggccgcagccccggagaggggaaaaggcgggccggacggcgccagcagccagcccgaggacgcgagcggcaggtgtgcacagaggttctccactttgttttctgaactcgcggtcaggatggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgctaaattatctgttgtcagttttgccccctcctccaatggtactccagaggttgaaacaacaagcctcaatgatgttactttaagcttactcccttcaaacgaaacagaaaaaactaaaatcactatagtaaaaaccttcaatgcttcaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgcaatagccgctttggaaagagtaaagattcgaccaatggaacactgctgctgttctgtcaggataccctgcccttcctccccagaagagttgggaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtgattcctttcttctaaaatcaaagcatgatgcttgacagtgtgaaatgtccaattttaccttttacacaatgtgagatgtatgaaaatcaactcattttattctcggcaacatctggagaagcataagctaattaagggcgatgattattattacaagaagaaaccaagacattacaccatggtttttagacatttctgatttggtttcttatctttcattttataagaaggttggttttaaacaatacactaagaatgactcctataaagaaaacaaaaaaaggtagtgaactttcagctaccttttaaagaggctaagttatctttgataacatcatataaagcaactgttgacttcagcctgttggtgagtttagttgtgcatgcctttgttgtatataagctaaattctagtgacccatgtgtcaaaaatcttacttctacatttttttgtatttattttctactgtgtaaatgtattcctttgtagaatcatggttgttttgtctcacgtgataattcagaaaatccttgctcgttccgcaaatcctaaagctccttttggagatgatataggatgtgaaatacagaaacctcagtgaaatcaagaaataatgatcccagccagactgagaaaatgtaagcagacagtgccacagttagctcatacagtgcctttgagcaagttaggaaaagatgcccccactgggcagacacagccctatgggtcatggtttgacaaacagagtgagagaccatattttagccccactcaccctcttgggtgcacgacctgtacagccaaacacagcatccaatatgaatacccatcccctgaccgcatccccagtagtcagattatagaatctgcaccaagatgtttagctttataccttggccacagagagggatgaactgtcatccagaccatgtgtcaggaaaattgtgaacgtagatgaggtacatacactgccgcttctcaaatccccagagcctttaggaacaggagagtagactaggattccttctcttaaaaaggtacatatatatggaaaaaaatcatattgccgttctttaaaaggcaactgcatggtacattgttgattgttatgactggtacactctggcccagccagagctataattgttttttaaatgtgtcttgaagaatgcacagtgacaaggggagtagctattgggaacagggaactgtcctacactgctattgttgctacatgtatcgagccttgattgctcctagttatatacagggtctatcttgcttcctacctacatctgcttgagcagtgcctcaagtacatccttattaggaacatttcaaaccccttttagttaagtctttcactaaggttctcttgcatatatttcaagtgaatgttggatctcagactaaccatagtaataatacacatttctgtgagtgctgacttgtctttgcaatatttcttttctgatttatttaattttcttgtatttatatgttaaaatcaaaaatgttaaaatcaatgaaataaatttgcagttaaga

SEQ ID NO: 23 - DNA, 4684 bp, Mus musculus
gccaaccggtcaccgggacccgcagatgcacacggagtttcctccctatttcctctgaacttcctgtcaggatgcttttctctggtgggcagtacagccctgttggcagacctgaagaggttttactgatatacaagatattccttgtcatcatttgttttcatgtcattctcgttacatccctgaaagaaaacggtaattccagtttgttatcaccatctgctgaatcatctcttgtcagtctcatcccctactccaatggtacaccagatgctgcttcagaagttttgtcgactttaaacaaaacagaaaaatctaaaatcactatagtaaaaaccttcaatgcatcaggagtcaaatcccagagaaatatctgcaatttgtcatctctttgcaatgactcagtattttttagaggtgagatagtgtttcaacatgatgaagaccacaatgttacccagaatcaagatacagctaatggcaccttcgctggagtcctgtctctaagtgaactgaagcgatcagagctcaacaaaactctacagaccttaagtgagacttactttatagtgtgtgctacagcagaggcccaaagcacggtaaactgtacattcacagtaaaactcaatgagaccatgaatgtgtgtgccatgatggttactttccaaactgtacagattcggccaatggaacagtgctgctgttccccgaggactccctgcccttcctcaccagaagagttagaaaaactacagtgtgaactgcaggatcccattgtctgtcttgctgatcaaccgcatggcccaccgttatcgtcttccagcaagcctgttgtacctcaggccaccattatttcccatgttgctagtgacttctctttggctgaaccccttgatcatgcccttatgaccccaagcacaccctctctgacacaagaaagtaaccttccatctcctcagcctacgatccccctggcttccagtcctgccactgacttgccagttcaatctgtagtggtctcttctttgcctcaaactgatctttcccacaccctgtcaccggtgcagtcctccattccctctcctaccacaccagccccatctgtccctacagaactggtcaccatcagcacacctcctggtgagacagttgtcaacactagcactgtttctgatctggaagcccaagtatcccagatggagaaagctttgtctttgggtagcttagagcctaatcttgcaggcgaaatggtaaaccgagtcagcaaactccttcactctccacctgccttgctagcccctctagctcaaaggttgctaaaagtggtagatgccattggcttacagctgaatttttcatctacaactatcagtctaacttcaccttctttggctcttgctgtgatcagagtgaatgccagtaatttcaataccacgacttttgcagcccaagacccaacaaatctccaggtttctctggaaaccccacctcctgagaatagtattggtgccattactctgccctcatcactgatgaataatttgccagctaatgatgtagaattggcttcaaggattcagttcaatttctttgaaacacccgccctgtttcaggatccttccctggagaacctcactctgataagctatgtcatatcatcaagtgtcacaaacatgaccatcaagaacttgacaagaaacgtgacagtcgcactgaaacacatcaacccaagtccggatgacttaactgtgaaatgtgtattctgggacttgggcagaaatggtggcaaaggaggttggtcatctgatggctgttccgtcaaagacaagagaatgaatgaaaccatctgtacctgtagccatcttacaagttttggcatcctattggacctatctcgaacatccttaccaccaagtcaaatgatggctctgacatttatcacgtatattggctgtgggctttcatcaatttttctgtcagttactcttgtaacctacatcgcctttgaaaagatccggagggattacccctccaaaatcctcatccagctgtgtgctgccctgcttctgctcaacctgatcttcctcctagactcctggattgcgctgtataatacccgaggtttctgcattgccgtggctgtatttcttcactattttctcttggtctcattcacatggatgggattagaagcattccacatgtacctagcactggtcaaggtgtttaatacttacatccgaaagtacatccttaaattctgcattgttggctggggcataccagctgtggttgtgtccatcgtcctgactatatccccagataactatgggattggatcctatggaaaattccccaatggcacaccagatgacttttgctggatcaacagcaatgtggtgttctatatcacggttgtgggatatttctgtgtgatatttctactgaacgtcagcatgttcatcgtggtcttggttcagctctgtcgaattaaaaagaagaagcagctgggagcccagcgcaaaactagtattcaagacctcaggagtatcgctggcctcacatttttactgggaattacttggggctttgccttctttgcctggggaccagttaatgtcaccttcatgtatctctttgccatctttaacaccttacaagggtttttcatattcattttttactgtgcagcaaaagagaatgtcagaaaacagtggaggcggtatctttgttgtggaaaattacggctggctgaaaattctgactggagtaaaactgctactaatggtttaaagaagcagactgtaaaccaaggagtatccagctcttcaaattccttacagtcaagctgtaactccactaactccaccacactcctagtgaatagtgattgctcagtgcacgcaagcgggaatggcaatgcatctaccgagaggaatggggtttctttcagtgttcagaatggagacgtgtgccttcatgatctcactggcaaacagcatatgtttagtgacaaagaggactcctgcaatggtaaaagccggatagcgctcagaaggacttcgaagcggggaagcttacactttatcgagcaaatgtgattctcttcttctaaaatcaaagcatgatgtggatggtgtgaagtgtccagttttcctttaacacaatgtgagatgtatgaaaagcgctggttttctattcagcaacctcgaaaggaacataagctaaatgagggcaaccagttactatcagaagaaaccacgactggttatggtttctagacgtttgcagtttggtttcttatctctcattttcaaaggcattggtgttaaaaccacagaataagatccaggaaagaaaaatgaaagtttgtttccactcacattttaaagaggctgttatccttgataacatcctaagaaagaaggaaaaaaagaaggaaagaaagaaagaaagagagagagagagagagaaagaaaaaagaaagaaagatgtttacttcagtctgttggtaagtttagttgtgcatgcctttgttgtatataagctaaattctagtgacccacatgtcaaaaaatcttctctttacctttttttatatttattttctactgtgtaaatttattcttttgtagaatcatggttgtgtcgccttctctaatgtgataacttaccagtgacttctaaacctcctttagaagatcatataggatgtgaaattcagaaacttcactgaaatcaagaaataatgataaaggcaatctgaacaattataaatagacaattagctcagaaagtgcctttgagcaatcaagaaaaggtgcccccactgggcagatatagccttgtgagcttatggtttggaaaacagtcgggccacattttagccctgatgatcctcctgggtaccttatgcagggtcctcatgaagggtccttatgcagggaatgacttgtatgactatatgtgacatgaaatagtgataccaatggccagacttcatccctaatgaccagattatagaatctctacaccatggttgttagaggctaaaactgtaatctaaaccatgttaggaaaatcgtgatggtagaaaagatacacactgccacttctcaaatacccagagcctttcaaaagaggggatcagagtaggattacttttccagagtgtatatatggcaaacaaagtaggtctttaaaagacgctgcatgatatgttattgttgcaactgctacattctggtccagtcatacttgtatgtatttttaaaaacatgtcaaatcttgaagagtgtatagtgacaagggggagcagctactgggaagagtgaattatcctgtgatattgttgtttgtatgtaataacatatatccaaccttgattgttcctagttctagagggcctatcctatttctcacctttaccagcttgagtggtgcctcaagcagtgtgattattaggaacatttcaaaatcctttagttaggtctttcactaaggctcccttgcatatatttcaaataaatgttgtatctgagactagccacagtagtaacatctgtttctgtgtgtgctcattgtctttgcaaaattcttctccaaattatttaattattttgtatttatatgttatgattgacaaaactgttaaaaaccaataaaaatcaatttgcagttaaaa

SEQ ID NO: 24 - DNA, 4636 bp, Mus musculus
gccaaccggtcaccgggacccgcagatgcacacggagtttcctccctatttcctctgaacttcctgtcaggatgcttttctctggtgggcagtacagccctgttggcagacctgaagaggttttactgatatacaagatattccttgtcatcatttgttttcatgtcattctcgttacatccctgaaagaaaacggtaattccagtttgttatcaccatctgatgctgcttcagaagttttgtcgactttaaacaaaacagaaaaatctaaaatcactatagtaaaaaccttcaatgcatcaggagtcaaatcccagagaaatatctgcaatttgtcatctctttgcaatgactcagtattttttagaggtgagatagtgtttcaacatgatgaagaccacaatgttacccagaatcaagatacagctaatggcaccttcgctggagtcctgtctctaagtgaactgaagcgatcagagctcaacaaaactctacagaccttaagtgagacttactttatagtgtgtgctacagcagaggcccaaagcacggtaaactgtacattcacagtaaaactcaatgagaccatgaatgtgtgtgccatgatggttactttccaaactgtacagattcggccaatggaacagtgctgctgttccccgaggactccctgcccttcctcaccagaagagttagaaaaactacagtgtgaactgcaggatcccattgtctgtcttgctgatcaaccgcatggcccaccgttatcgtcttccagcaagcctgttgtacctcaggccaccattatttcccatgttgctagtgacttctctttggctgaaccccttgatcatgcccttatgaccccaagcacaccctctctgacacaagaaagtaaccttccatctcctcagcctacgatccccctggcttccagtcctgccactgacttgccagttcaatctgtagtggtctcttctttgcctcaaactgatctttcccacaccctgtcaccggtgcagtcctccattccctctcctaccacaccagccccatctgtccctacagaactggtcaccatcagcacacctcctggtgagacagttgtcaacactagcactgtttctgatctggaagcccaagtatcccagatggagaaagctttgtctttgggtagcttagagcctaatcttgcaggcgaaatggtaaaccgagtcagcaaactccttcactctccacctgccttgctagcccctctagctcaaaggttgctaaaagtggtagatgccattggcttacagctgaatttttcatctacaactatcagtctaacttcaccttctttggctcttgctgtgatcagagtgaatgccagtaatttcaataccacgacttttgcagcccaagacccaacaaatctccaggtttctctggaaaccccacctcctgagaatagtattggtgccattactctgccctcatcactgatgaataatttgccagctaatgatgtagaattggcttcaaggattcagttcaatttctttgaaacacccgccctgtttcaggatccttccctggagaacctcactctgataagctatgtcatatcatcaagtgtcacaaacatgaccatcaagaacttgacaagaaacgtgacagtcgcactgaaacacatcaacccaagtccggatgacttaactgtgaaatgtgtattctgggacttgggcagaaatggtggcaaaggaggttggtcatctgatggctgttccgtcaaagacaagagaatgaatgaaaccatctgtacctgtagccatcttacaagttttggcatcctattggacctatctcgaacatccttaccaccaagtcaaatgatggctctgacatttatcacgtatattggctgtgggctttcatcaatttttctgtcagttactcttgtaacctacatcgcctttgaaaagatccggagggattacccctccaaaatcctcatccagctgtgtgctgccctgcttctgctcaacctgatcttcctcctagactcctggattgcgctgtataatacccgaggtttctgcattgccgtggctgtatttcttcactattttctcttggtctcattcacatggatgggattagaagcattccacatgtacctagcactggtcaaggtgtttaatacttacatccgaaagtacatccttaaattctgcattgttggctggggcataccagctgtggttgtgtccatcgtcctgactatatccccagataactatgggattggatcctatggaaaattccccaatggcacaccagatgacttttgctggatcaacagcaatgtggtgttctatatcacggttgtgggatatttctgtgtgatatttctactgaacgtcagcatgttcatcgtggtcttggttcagctctgtcgaattaaaaagaagaagcagctgggagcccagcgcaaaactagtattcaagacctcaggagtatcgctggcctcacatttttactgggaattacttggggctttgccttctttgcctggggaccagttaatgtcaccttcatgtatctctttgccatctttaacaccttacaagggtttttcatattcattttttactgtgcagcaaaagagaatgtcagaaaacagtggaggcggtatctttgttgtggaaaattacggctggctgaaaattctgactggagtaaaactgctactaatggtttaaagaagcagactgtaaaccaaggagtatccagctcttcaaattccttacagtcaagctgtaactccactaactccaccacactcctagtgaatagtgattgctcagtgcacgcaagcgggaatggcaatgcatctaccgagaggaatggggtttctttcagtgttcagaatggagacgtgtgccttcatgatctcactggcaaacagcatatgtttagtgacaaagaggactcctgcaatggtaaaagccggatagcgctcagaaggacttcgaagcggggaagcttacactttatcgagcaaatgtgattctcttcttctaaaatcaaagcatgatgtggatggtgtgaagtgtccagttttcctttaacacaatgtgagatgtatgaaaagcgctggttttctattcagcaacctcgaaaggaacataagctaaatgagggcaaccagttactatcagaagaaaccacgactggttatggtttctagacgtttgcagtttggtttcttatctctcattttcaaaggcattggtgttaaaaccacagaataagatccaggaaagaaaaatgaaagtttgtttccactcacattttaaagaggctgttatccttgataacatcctaagaaagaaggaaaaaaagaaggaaagaaagaaagaaagagagagagagagagagaaagaaaaaagaaagaaagatgtttacttcagtctgttggtaagtttagttgtgcatgcctttgttgtatataagctaaattctagtgacccacatgtcaaaaaatcttctctttacctttttttatatttattttctactgtgtaaatttattcttttgtagaatcatggttgtgtcgccttctctaatgtgataacttaccagtgacttctaaacctcctttagaagatcatataggatgtgaaattcagaaacttcactgaaatcaagaaataatgataaaggcaatctgaacaattataaatagacaattagctcagaaagtgcctttgagcaatcaagaaaaggtgcccccactgggcagatatagccttgtgagcttatggtttggaaaacagtcgggccacattttagccctgatgatcctcctgggtaccttatgcagggtcctcatgaagggtccttatgcagggaatgacttgtatgactatatgtgacatgaaatagtgataccaatggccagacttcatccctaatgaccagattatagaatctctacaccatggttgttagaggctaaaactgtaatctaaaccatgttaggaaaatcgtgatggtagaaaagatacacactgccacttctcaaatacccagagcctttcaaaagaggggatcagagtaggattacttttccagagtgtatatatggcaaacaaagtaggtctttaaaagacgctgcatgatatgttattgttgcaactgctacattctggtccagtcatacttgtatgtatttttaaaaacatgtcaaatcttgaagagtgtatagtgacaagggggagcagctactgggaagagtgaattatcctgtgatattgttgtttgtatgtaataacatatatccaaccttgattgttcctagttctagagggcctatcctatttctcacctttaccagcttgagtggtgcctcaagcagtgtgattattaggaacatttcaaaatcctttagttaggtctttcactaaggctcccttgcatatatttcaaataaatgttgtatctgagactagccacagtagtaacatctgtttctgtgtgtgctcattgtctttgcaaaattcttctccaaattatttaattattttgtatttatatgttatgattgacaaaactgttaaaaaccaataaaaatcaatttgcagttaaaa

SEQ ID NO: 25 - DNA, 4675 bp, Mus musculus
gccaaccggtcaccgggacccgcagatgcacacggagtttcctccctatttcctctgaacttcctgtcaggatgcttttctctggtgggcagtacagccctgttggcagacctgaagaggttttactgatatacaagatattccttgtcatcatttgttttcatgtcattctcgttacatccctgaaagaaaacggtaattccagtttgttatcaccatctgctgaatcatctcttgtcagtctcatcccctactccaatgatgctgcttcagaagttttgtcgactttaaacaaaacagaaaaatctaaaatcactatagtaaaaaccttcaatgcatcaggagtcaaatcccagagaaatatctgcaatttgtcatctctttgcaatgactcagtattttttagaggtgagatagtgtttcaacatgatgaagaccacaatgttacccagaatcaagatacagctaatggcaccttcgctggagtcctgtctctaagtgaactgaagcgatcagagctcaacaaaactctacagaccttaagtgagacttactttatagtgtgtgctacagcagaggcccaaagcacggtaaactgtacattcacagtaaaactcaatgagaccatgaatgtgtgtgccatgatggttactttccaaactgtacagattcggccaatggaacagtgctgctgttccccgaggactccctgcccttcctcaccagaagagttagaaaaactacagtgtgaactgcaggatcccattgtctgtcttgctgatcaaccgcatggcccaccgttatcgtcttccagcaagcctgttgtacctcaggccaccattatttcccatgttgctagtgacttctctttggctgaaccccttgatcatgcccttatgaccccaagcacaccctctctgacacaagaaagtaaccttccatctcctcagcctacgatccccctggcttccagtcctgccactgacttgccagttcaatctgtagtggtctcttctttgcctcaaactgatctttcccacaccctgtcaccggtgcagtcctccattccctctcctaccacaccagccccatctgtccctacagaactggtcaccatcagcacacctcctggtgagacagttgtcaacactagcactgtttctgatctggaagcccaagtatcccagatggagaaagctttgtctttgggtagcttagagcctaatcttgcaggcgaaatggtaaaccgagtcagcaaactccttcactctccacctgccttgctagcccctctagctcaaaggttgctaaaagtggtagatgccattggcttacagctgaatttttcatctacaactatcagtctaacttcaccttctttggctcttgctgtgatcagagtgaatgccagtaatttcaataccacgacttttgcagcccaagacccaacaaatctccaggtttctctggaaaccccacctcctgagaatagtattggtgccattactctgccctcatcactgatgaataatttgccagctaatgatgtagaattggcttcaaggattcagttcaatttctttgaaacacccgccctgtttcaggatccttccctggagaacctcactctgataagctatgtcatatcatcaagtgtcacaaacatgaccatcaagaacttgacaagaaacgtgacagtcgcactgaaacacatcaacccaagtccggatgacttaactgtgaaatgtgtattctgggacttgggcagaaatggtggcaaaggaggttggtcatctgatggctgttccgtcaaagacaagagaatgaatgaaaccatctgtacctgtagccatcttacaagttttggcatcctattggacctatctcgaacatccttaccaccaagtcaaatgatggctctgacatttatcacgtatattggctgtgggctttcatcaatttttctgtcagttactcttgtaacctacatcgcctttgaaaagatccggagggattacccctccaaaatcctcatccagctgtgtgctgccctgcttctgctcaacctgatcttcctcctagactcctggattgcgctgtataatacccgaggtttctgcattgccgtggctgtatttcttcactattttctcttggtctcattcacatggatgggattagaagcattccacatgtacctagcactggtcaaggtgtttaatacttacatccgaaagtacatccttaaattctgcattgttggctggggcataccagctgtggttgtgtccatcgtcctgactatatccccagataactatgggattggatcctatggaaaattccccaatggcacaccagatgacttttgctggatcaacagcaatgtggtgttctatatcacggttgtgggatatttctgtgtgatatttctactgaacgtcagcatgttcatcgtggtcttggttcagctctgtcgaattaaaaagaagaagcagctgggagcccagcgcaaaactagtattcaagacctcaggagtatcgctggcctcacatttttactgggaattacttggggctttgccttctttgcctggggaccagttaatgtcaccttcatgtatctctttgccatctttaacaccttacaagggtttttcatattcattttttactgtgcagcaaaagagaatgtcagaaaacagtggaggcggtatctttgttgtggaaaattacggctggctgaaaattctgactggagtaaaactgctactaatggtttaaagaagcagactgtaaaccaaggagtatccagctcttcaaattccttacagtcaagctgtaactccactaactccaccacactcctagtgaatagtgattgctcagtgcacgcaagcgggaatggcaatgcatctaccgagaggaatggggtttctttcagtgttcagaatggagacgtgtgccttcatgatctcactggcaaacagcatatgtttagtgacaaagaggactcctgcaatggtaaaagccggatagcgctcagaaggacttcgaagcggggaagcttacactttatcgagcaaatgtgattctcttcttctaaaatcaaagcatgatgtggatggtgtgaagtgtccagttttcctttaacacaatgtgagatgtatgaaaagcgctggttttctattcagcaacctcgaaaggaacataagctaaatgagggcaaccagttactatcagaagaaaccacgactggttatggtttctagacgtttgcagtttggtttcttatctctcattttcaaaggcattggtgttaaaaccacagaataagatccaggaaagaaaaatgaaagtttgtttccactcacattttaaagaggctgttatccttgataacatcctaagaaagaaggaaaaaaagaaggaaagaaagaaagaaagagagagagagagagagaaagaaaaaagaaagaaagatgtttacttcagtctgttggtaagtttagttgtgcatgcctttgttgtatataagctaaattctagtgacccacatgtcaaaaaatcttctctttacctttttttatatttattttctactgtgtaaatttattcttttgtagaatcatggttgtgtcgccttctctaatgtgataacttaccagtgacttctaaacctcctttagaagatcatataggatgtgaaattcagaaacttcactgaaatcaagaaataatgataaaggcaatctgaacaattataaatagacaattagctcagaaagtgcctttgagcaatcaagaaaaggtgcccccactgggcagatatagccttgtgagcttatggtttggaaaacagtcgggccacattttagccctgatgatcctcctgggtaccttatgcagggtcctcatgaagggtccttatgcagggaatgacttgtatgactatatgtgacatgaaatagtgataccaatggccagacttcatccctaatgaccagattatagaatctctacaccatggttgttagaggctaaaactgtaatctaaaccatgttaggaaaatcgtgatggtagaaaagatacacactgccacttctcaaatacccagagcctttcaaaagaggggatcagagtaggattacttttccagagtgtatatatggcaaacaaagtaggtctttaaaagacgctgcatgatatgttattgttgcaactgctacattctggtccagtcatacttgtatgtatttttaaaaacatgtcaaatcttgaagagtgtatagtgacaagggggagcagctactgggaagagtgaattatcctgtgatattgttgtttgtatgtaataacatatatccaaccttgattgttcctagttctagagggcctatcctatttctcacctttaccagcttgagtggtgcctcaagcagtgtgattattaggaacatttcaaaatcctttagttaggtctttcactaaggctcccttgcatatatttcaaataaatgttgtatctgagactagccacagtagtaacatctgtttctgtgtgtgctcattgtctttgcaaaattcttctccaaattatttaattattttgtatttatatgttatgattgacaaaactgttaaaaaccaataaaaatcaatttgcagttaaaa

SEQ ID NO: 26 - DNA, 4602 bp, Mus musculus
gccaaccggtcaccgggacccgcagatgcacacggagtttcctccctatttcctctgaacttcctgtcaggatgcttttctctggtgggcagtacagccctgttggcagacctgaagaggttttactgatatacaagatattccttgtcatcatttgttttcatgtcattctcgttacatccctgaaagatgctgcttcagaagttttgtcgactttaaacaaaacagaaaaatctaaaatcactatagtaaaaaccttcaatgcatcaggagtcaaatcccagagaaatatctgcaatttgtcatctctttgcaatgactcagtattttttagaggtgagatagtgtttcaacatgatgaagaccacaatgttacccagaatcaagatacagctaatggaccttcgctggagtcctgtctctaagtgaactgaagcgatcagagctcaacaaaactctacagaccttaagtgagacttactttatagtgtgtgctacagcagaggcccaaagcacggtaaactgtacattcacagtaaaactcaatgagaccatgaatgtgtgtgccatgatggttactttccaaactgtacagattcggccaatggaacagtgctgctgttccccgaggactccctgcccttcctcaccagaagagttagaaaaactacagtgtgaactgcaggatcccattgtctgtcttgctgatcaaccgcatggcccaccgttatcgtcttccagcaagcctgttgtacctcaggccaccattatttcccatgttgctagtgacttctctttggctgaaccccttgatcatgcccttatgaccccaagcacaccctctctgacacaagaaagtaaccttccatctcctcagcctacgatccccctggcttccagtcctgccactgacttgccagttcaatctgtagtggtctcttctttgcctcaaactgatctttcccacaccctgtcaccggtgcagtcctccattccctctcctaccacaccagccccatctgtccctacagaactggtcaccatcagcacacctcctggtgagacagttgtcaacactagcactgtttctgatctggaagcccaagtatcccagatggagaaagctttgtctttgggtagcttagagcctaatcttgcaggcgaaatggtaaaccgagtcagcaaactccttcactctccacctgccttgctagcccctctagctcaaaggttgctaaaagtggtagatgccattggcttacagctgaatttttcatctacaactatcagtctaacttcaccttctttggctcttgctgtgatcagagtgaatgccagtaatttcaataccacgacttttgcagcccaagacccaacaaatctccaggtttctctggaaaccccacctcctgagaatagtattggtgccattactctgccctcatcactgatgaataatttgccagctaatgatgtagaattggcttcaaggattcagttcaatttctttgaaacacccgccctgtttcaggatccttccctggagaacctcactctgataagctatgtcatatcatcaagtgtcacaaacatgaccatcaagaacttgacaagaaacgtgacagtcgcactgaaacacatcaacccaagtccggatgacttaactgtgaaatgtgtattctgggacttgggcagaaatggtggcaaaggaggttggtcatctgatggctgttccgtcaaagacaagagaatgaatgaaaccatctgtacctgtagccatcttacaagttttggcatcctattggacctatctcgaacatccttaccaccaagtcaaatgatggctctgacatttatcacgtatattggctgtgggctttcatcaatttttctgtcagttactcttgtaacctacatcgcctttgaaaagatccggagggattacccctccaaaatcctcatccagctgtgtgctgccctgcttctgctcaacctgatcttcctcctagactcctggattgcgctgtataatacccgaggtttctgcattgccgtggctgtatttcttcactattttctcttggtctcattcacatggatgggattagaagcattccacatgtacctagcactggtcaaggtgtttaatacttacatccgaaagtacatccttaaattctgcattgttggctggggcataccagctgtggttgtgtccatcgtcctgactatatccccagataactatgggattggatcctatggaaaattccccaatggcacaccagatgacttttgctggatcaacagcaatgtggtgttctatatcacggttgtgggatatttctgtgtgatatttctactgaacgtcagcatgttcatcgtggtcttggttcagctctgtcgaattaaaaagaagaagcagctgggagcccagcgcaaaactagtattcaagacctcaggagtatcgctggcctcacatttttactgggaattacttggggctttgccttctttgcctggggaccagttaatgtcaccttcatgtatctctttgccatctttaacaccttacaagggtttttcatattcattttttactgtgcagcaaaagagaatgtcagaaaacagtggaggcggtatctttgttgtggaaaattacggctggctgaaaattctgactggagtaaaactgctactaatggtttaaagaagcagactgtaaaccaaggagtatccagctcttcaaattccttacagtcaagctgtaactccactaactccaccacactcctagtgaatagtgattgctcagtgcacgcaagcgggaatggcaatgcatctaccgagaggaatggggtttctttcagtgttcagaatggagacgtgtgccttcatgatctcactggcaaacagcatatgtttagtgacaaagaggactcctgcaatggtaaaagccggatagcgctcagaaggacttcgaagcggggaagcttacactttatcgagcaaatgtgattctcttcttctaaaatcaaagcatgatgtggatggtgtgaagtgtccagttttcctttaacacaatgtgagatgtatgaaaagcgctggttttctattcagcaacctcgaaaggaacataagctaaatgagggcaaccagttactatcagaagaaaccacgactggttatggtttctagacgtttgcagtttggtttcttatctctcattttcaaaggcattggtgttaaaaccacagaataagatccaggaaagaaaaatgaaagtttgtttccactcacattttaaagaggctgttatccttgataacatcctaagaaagaaggaaaaaaagaaggaaagaaagaaagaaagagagagagagagagagaaagaaaaaagaaagaaagatgtttacttcagtctgttggtaagtttagttgtgcatgcctttgttgtatataagctaaattctagtgacccacatgtcaaaaaatcttctctttacctttttttatatttattttctactgtgtaaatttattcttttgtagaatcatggttgtgtcgccttctctaatgtgataacttaccagtgacttctaaacctcctttagaagatcatataggatgtgaaattcagaaacttcactgaaatcaagaaataatgataaaggcaatctgaacaattataaatagacaattagctcagaaagtgcctttgagcaatcaagaaaaggtgcccccactgggcagatatagccttgtgagcttatggtttggaaaacagtcgggccacattttagccctgatgatcctcctgggtaccttatgcagggtcctcatgaagggtccttatgcagggaatgacttgtatgactatatgtgacatgaaatagtgataccaatggccagacttcatccctaatgaccagattatagaatctctacaccatggttgttagaggctaaaactgtaatctaaaccatgttaggaaaatcgtgatggtagaaaagatacacactgccacttctcaaatacccagagcctttcaaaagaggggatcagagtaggattacttttccagagtgtatatatggcaaacaaagtaggtctttaaaagacgctgcatgatatgttattgttgcaactgctacattctggtccagtcatacttgtatgtatttttaaaaacatgtcaaatcttgaagagtgtatagtgacaagggggagcagctactgggaagagtgaattatcctgtgatattgttgtttgtatgtaataacatatatccaaccttgattgttcctagttctagagggcctatcctatttctcacctttaccagcttgagtggtgcctcaagcagtgtgattattaggaacatttcaaaatcctttagttaggtctttcactaaggctcccttgcatatatttcaaataaatgttgtatctgagactagccacagtagtaacatctgtttctgtgtgtgctcattgtctttgcaaaattcttctccaaattatttaattattttgtatttatatgttatgattgacaaaactgttaaaaaccaataaaaatcaatttgcagttaaaa

SEQ ID NO: 27 - DNA, 4614 bp, Rattus norvegicus
ccaggacctgcagatgtgcacagagtctcctccctcttttctctgaacttcctgtcaggatgcttttctctgggggtcagtacagccctgttggcaggcctgaagaggttttactgatatacaagatattccttgtcatcatttgttttcatgccattcttgttacatccctgaaagaaaatgctggtaattccagtttgttgtcaccatctgctgaatcgtctcttgtcagtcttgttccctactccaatggtacaccagatgctgcttccgaagtattgtcgactttaaacagaacagaaaaatctaaaatcactatattaaaaaccttcaatgcatcaggagtcaaatcccagagaaatatctgcaatttgtcatctatttgcagtgactcagtgttttttagaggtgagatagtgtttcaacatgatgaccactacaacgttactcagaatcaagatatagttaacagcaccttcgctggagtcctgtctctaagcgaactgaagcgaacagaactcaacaaaactctacagaccttaagtgagacttactttatagtgtgtgctaccgcagaggcccaaaacacattaaactgtacattcacagtaaaactgaatgagaccatgaatgtgtgtgccatgatggttactttcaaaagtgtacagattcggccaatggaacagtgctgctgttcccccaggactccctgcccttcctcgccggaagagttagaaaaactacagtgtgatctgcaggatcccattgtctgccttgccgatcaaccacatggcccaccagtatcttcttccagcaagcctgtgccagttgtacctcaggccaccattttttcccacgttgctagtgacttctctttggctgaaccccttgatcatgcccttatgacctcaagcacaccctctctggcacaagagacacgccttccatctcctcagcctacaatctccctgacttccagtcctgccattgacttgcctgttcaacatgtagtggcttcttcctccttgccccaaactgatctttcccacaccctatcacctgtgcagtcctccattccctctcctaccacagcagctccatctgtccctgaaaaagtggtcgccatcagcacacctcctggcgagacagttgtcaacacgagtagtgttcctgatctggaagcccaagtatctcaaatggagaaagctttgtctttgggcagcttggaacctaatcttgcgggtgaaatggtgaaccgagtcagcaaactccttcactctccacttgccttgctagcccctctagctcaaaggttgctaaaagtggtagatgccattggcttacagctgaatttttcatctacaactatcagtctaacttctccttctttggctcttgctgtgatcagagtgaatgccagtaatttcaatacaacaacctttgcagcccaagacccagcaaatctccaggtgtctctggaagcccaggctcctaagaatagtattggcgccattactctaccctcatcgctgatgagtaatttgccagctagtgaggtagaattggcttcgagggttcagttcaatttctttgaaacacctgccctatttcaggacccctccctggagaacctctctctgataagctatgtcatatcatcaagtgtcacaaacatgaccatcaagaacttgacaagaaacgtgacagttgcactgaagcacatcaacccaagtcaggatgacttaactgtcaaatgtgttttctgggacttgaacagaaatggtggcagaggaggttggtcatctgatggctgttccgtcaaagagaagaggatgaacgaaactatctgtacctgtagccaccttacaagttttggcatcctattggatctatctcggacatccttaccaccaagtcaaatgatggctctgacatttatcacgtatattggctgtgggctttcatcaatttttctgtcagttactcttgtaacctatatagcctttgaaaagatccggagggattacccttccaaaatcctcattcaactgtgtgctgccctgcttctgctcaacctggtcttcctcttagactcctggattgcactgtataatgcccgaggtttctgcatctccgtggctgtatttcttcactattttctcttggtatcattcacatggatgggcttagaagcatttcatatgtacctagcactggttaaggtttttaatacttacatccgaaagtacatccttaaattctgcattgttggctgggggataccagctgtggttgtatccatcgtcctgactatatccccagataattatgggattggatcttatgggaaattccccaatggcacaccagatgacttttgctggatcaacagcagtgtggtgttctatatcacggttgtgggatatttctgtgtgatatttctactgaacgtcagcatgttcattgtggtcttggttcagctctgtcgaattaaaaagaagaagcaactgggagcgcagcgcaaaactagtattcaagacctccggagtattgctggcctcacatttttactgggaattacttggggctttgccttctttgcctggggaccagttaatctcaccttcatgtatctctttgccatctttaacaccttacagggctttttcatattcatcttttactgtgcagcaaaagagaatgtcagaaaacagtggaggcggtatctttgttgtggaaaattacgactggctgaaaactctgactggagtaaaactgctactaatggtttaaagaagcagactgtaaatcaaggagtatccagctcttcaaattccttacagtcaagctgtaactccactaactccactacactcctagtgaatagtgattgctcagtgcacgcaagcgggaatggcaatgcatctaccgagaggaatggggtttctttcagtgttcagaatggagacgtgtgccttcatgatctcactgggaaacagcacatgtttagtgacaaagaggactcctgcaatggcaaaagccggatggcactcagaaggacttcaaagcggggaagcttacactttatcgagcaaatgtgattcgcttcttctaaaatcaaagcatgatgttgatggtgtgaagtgtccagttttccttttatacaatgtgagatatatgaaaatcgctggttttctactcagcaacctcgaaaggaacataagctaattgagggcaacagtttactgtcggaagaaggaaactatgacatgattatggttcctagacattctcaatttcgtttattatctcccatttgtgaggggattggggttagaatcacagaataagatccaagaaaagaaaaatgaaagtttgtttccagtcacattttaaagaggctgttatccttgataacatcctttaaaaaatgtttacttcagtctgttggtgagttgagttgtgcatgcctttgttgtatataagctaagttctaatgacccacatgtcaaaaatcgtatctctatctttttttatatttattttctactgtgtaaatttattcttttgtagaatcatggttgtgtcgttttctctaatgtgataacttaccagtgacttctaaacctcctttcggagatcatataggatgtgaaattcagaaacttcactgaaatcaagaaataatgataaaggcagactgaacaattataaatagacagtgccacaatcagcttagctatcagcctctgagctatcaaggaaaggtgtccccattggacggacatagccttatgagcttatggtttgggaaaaagtggggtcacattttcgccctgatgatcctcttgagtaccttattcagggcccttatgtagggtctttatgcagggaatgacttatatgactatattggacactaaatagggatacctatgaccagacttcatccctaatgatcatattatagaatctctacaccatggttgttagaggataaaattgtaatctaaaccatgtagggaagactgtgaatgtgggaaagatatacactgccacttctcaaatacccagagcctttgaaaagaggaaatcagagtaggattacttttccagagtgtatatatggcaaacgagttaggtctttaaaagacaactgcatggtacgtcattgttgcaactgctacattctggtcctacttgtttgtattcttaaaacatgtcaaatattgaagagtgtatagtgacaagggggagcagctactgggaagagtgaactgtcgtgtgatattgttgtttgtaggtaataatatatatccaactctgattgttccgagtcctagagcatctatcctgtttctcaccgcttgagtggtgtctcaagcccaggatgtttattaggaacatttcaagatcctttccttaggtctttcactaagctcccttgcatatatttcaagtagatgttgtatcccagactaaccacagtagtaacgtctgtttctctgtgtgctcgttggtctttgcaaaatttcttctccaaattatttaattattttgtatttatatgttacaattgacaaaaatgataaaatcaataaaataaatttgcac

SEQ ID NO: 28 - DNA, 4530 bp, Rattus norvegicus
ccaggacctgcagatgtgcacagagtctcctccctcttttctctgaacttcctgtcaggatgcttttctctgggggtcagtacagccctgttggcaggcctgaagaggttttactgatatacaagatattccttgtcatcatttgttttcatgccattcttgttacatccctgaaagatgctgcttccgaagtattgtcgactttaaacagaacagaaaaatctaaaatcactatattaaaaaccttcaatgcatcaggagtcaaatcccagagaaatatctgcaatttgtcatctatttgcagtgactcagtgttttttagaggtgagatagtgtttcaacatgatgaccactacaacgttactcagaatcaagatatagttaacagcaccttcgctggagtcctgtctctaagcgaactgaagcgaacagaactcaacaaaactctacagaccttaagtgagacttactttatagtgtgtgctaccgcagaggcccaaaacacattaaactgtacattcacagtaaaactgaatgagaccatgaatgtgtgtgccatgatggttactttcaaaagtgtacagattcggccaatggaacagtgctgctgttcccccaggactccctgcccttcctcgccggaagagttagaaaaactacagtgtgatctgcaggatcccattgtctgccttgccgatcaaccacatggcccaccagtatcttcttccagcaagcctgtgccagttgtacctcaggccaccattttttcccacgttgctagtgacttctctttggctgaaccccttgatcatgcccttatgacctcaagcacaccctctctggcacaagagacacgccttccatctcctcagcctacaatctccctgacttccagtcctgccattgacttgcctgttcaacatgtagtggcttcttcctccttgccccaaactgatctttcccacaccctatcacctgtgcagtcctccattccctctcctaccacagcagctccatctgtccctgaaaaagtggtcgccatcagcacacctcctggcgagacagttgtcaacacgagtagtgttcctgatctggaagcccaagtatctcaaatggagaaagctttgtctttgggcagcttggaacctaatcttgcgggtgaaatggtgaaccgagtcagcaaactccttcactctccacttgccttgctagcccctctagctcaaaggttgctaaaagtggtagatgccattggcttacagctgaatttttcatctacaactatcagtctaacttctccttctttggctcttgctgtgatcagagtgaatgccagtaatttcaatacaacaacctttgcagcccaagacccagcaaatctccaggtgtctctggaagcccaggctcctaagaatagtattggcgccattactctaccctcatcgctgatgagtaatttgccagctagtgaggtagaattggcttcgagggttcagttcaatttctttgaaacacctgccctatttcaggacccctccctggagaacctctctctgataagctatgtcatatcatcaagtgtcacaaacatgaccatcaagaacttgacaagaaacgtgacagttgcactgaagcacatcaacccaagtcaggatgacttaactgtcaaatgtgttttctgggacttgaacagaaatggtggcagaggaggttggtcatctgatggctgttccgtcaaagagaagaggatgaacgaaactatctgtacctgtagccaccttacaagttttggcatcctattggatctatctcggacatccttaccaccaagtcaaatgatggctctgacatttatcacgtatattggctgtgggctttcatcaatttttctgtcagttactcttgtaacctatatagcctttgaaaagatccggagggattacccttccaaaatcctcattcaactgtgtgctgccctgcttctgctcaacctggtcttcctcttagactcctggattgcactgtataatgcccgaggtttctgcatctccgtggctgtatttcttcactattttctcttggtatcattcacatggatgggcttagaagcatttcatatgtacctagcactggttaaggtttttaatacttacatccgaaagtacatccttaaattctgcattgttggctgggggataccagctgtggttgtatccatcgtcctgactatatccccagataattatgggattggatcttatgggaaattccccaatggcacaccagatgacttttgctggatcaacagcagtgtggtgttctatatcacggttgtgggatatttctgtgtgatatttctactgaacgtcagcatgttcattgtggtcttggttcagctctgtcgaattaaaaagaagaagcaactgggagcgcagcgcaaaactagtattcaagacctccggagtattgctggcctcacatttttactgggaattacttggggctttgccttctttgcctggggaccagttaatctcaccttcatgtatctctttgccatctttaacaccttacagggctttttcatattcatcttttactgtgcagcaaaagagaatgtcagaaaacagtggaggcggtatctttgttgtggaaaattacgactggctgaaaactctgactggagtaaaactgctactaatggtttaaagaagcagactgtaaatcaaggagtatccagctcttcaaattccttacagtcaagctgtaactccactaactccactacactcctagtgaatagtgattgctcagtgcacgcaagcgggaatggcaatgcatctaccgagaggaatggggtttctttcagtgttcagaatggagacgtgtgccttcatgatctcactgggaaacagcacatgtttagtgacaaagaggactcctgcaatggcaaaagccggatggcactcagaaggacttcaaagcggggaagcttacactttatcgagcaaatgtgattcgcttcttctaaaatcaaagcatgatgttgatggtgtgaagtgtccagttttccttttatacaatgtgagatatatgaaaatcgctggttttctactcagcaacctcgaaaggaacataagctaattgagggcaacagtttactgtcggaagaaggaaactatgacatgattatggttcctagacattctcaatttcgtttattatctcccatttgtgaggggattggggttagaatcacagaataagatccaagaaaagaaaaatgaaagtttgtttccagtcacattttaaagaggctgttatccttgataacatcctttaaaaaatgtttacttcagtctgttggtgagttgagttgtgcatgcctttgttgtatataagctaagttctaatgacccacatgtcaaaaatcgtatctctatctttttttatatttattttctactgtgtaaatttattcttttgtagaatcatggttgtgtcgttttctctaatgtgataacttaccagtgacttctaaacctcctttcggagatcatataggatgtgaaattcagaaacttcactgaaatcaagaaataatgataaaggcagactgaacaattataaatagacagtgccacaatcagcttagctatcagcctctgagctatcaaggaaaggtgtccccattggacggacatagccttatgagcttatggtttgggaaaaagtggggtcacattttcgccctgatgatcctcttgagtaccttattcagggcccttatgtagggtctttatgcagggaatgacttatatgactatattggacactaaatagggatacctatgaccagacttcatccctaatgatcatattatagaatctctacaccatggttgttagaggataaaattgtaatctaaaccatgtagggaagactgtgaatgtgggaaagatatacactgccacttctcaaatacccagagcctttgaaaagaggaaatcagagtaggattacttttccagagtgtatatatggcaaacgagttaggtctttaaaagacaactgcatggtacgtcattgttgcaactgctacattctggtcctacttgtttgtattcttaaaacatgtcaaatattgaagagtgtatagtgacaagggggagcagctactgggaagagtgaactgtcgtgtgatattgttgtttgtaggtaataatatatatccaactctgattgttccgagtcctagagcatctatcctgtttctcaccgcttgagtggtgtctcaagcccaggatgtttattaggaacatttcaagatcctttccttaggtctttcactaagctcccttgcatatatttcaagtagatgttgtatcccagactaaccacagtagtaacgtctgtttctctgtgtgctcgttggtctttgcaaaatttcttctccaaattatttaattattttgtatttatatgttacaattgacaaaaatgataaaatcaataaaataaatttgcac

SEQ ID NO: 29 - DNA, 4530 bp, Rattus norvegicus; identical to SEQ ID NO: 28

SEQ ID NO: 30 - protein, 1017 aa, Homo sapiens [sequence omitted]

SEQ ID NO: 31 - protein, 1009 aa, Mus musculus
MLFSGGQYSPVGRPEEVLLIYKIFLVIICFHVILVTSLKENGNSSLLSPSAESSLVSLIPYSNGTPDAASEVLSTLNKTEKSKITIVKTFNASGVKSQRNICNLSSLCNDSVFFRGEIVFQHDEDHNVTQNQDTANGTFAGVLSLSELKRSELNKTLQTLSETYFIVCATAEAQSTVNCTFTVKLNETMNVCAMMVTFQTVQIRPMEQCCCSPRTPCPSSPEELEKLQCELQDPIVCLADQPHGPPLSSSSKPVVPQATIISHVASDFSLAEPLDHALMTPSTPSLTQESNLPSPQPTIPLASSPATDLPVQSVVVSSLPQTDLSHTLSPVQSSIPSPTTPAPSVPTELVTISTPPGETVVNTSTVSDLEAQVSQMEKALSLGSLEPNLAGEMVNRVSKLLHSPPALLAPLAQRLLKVVDAIGLQLNFSSTTISLTSPSLALAVIRVNASNFNTTTFAAQDPTNLQVSLETPPPENSIGAITLPSSLMNNLPANDVELASRIQFNFFETPALFQDPSLENLTLISYVISSSVTNMTIKNLTRNVTVALKHINPSPDDLTVKCVFWDLGRNGGKGGWSSDGCSVKDKRMNETICTCSHLTSFGILLDLSRTSLPPSQMMALTFITYIGCGLSSIFLSVTLVTYIAFEKIRRDYPSKILIQLCAALLLLNLIFLLDSWIALYNTRGFCIAVAVFLHYFLLVSFTWMGLEAFHMYLALVKVFNTYIRKYILKFCIVGWGIPAVVVSIVLTISPDNYGIGSYGKFPNGTPDDFCWINSNVVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRIALRRTSKRGSLHFIEQM

SEQ ID NO: 32 - protein, 1013 aa, Rattus norvegicus
MLFSGGQYSPVGRPEEVLLIYKIFLVIICFHAILVTSLKENAGNSSLLSPSAESSLVSLVPYSNGTPDAASEVLSTLNRTEKSKITILKTFNASGVKSQRNICNLSSICSDSVFFRGEIVFQHDDHYNVTQNQDIVNSTFAGVLSLSELKRTELNKTLQTLSETYFIVCATAEAQNTLNCTFTVKLNETMNVCAMMVTFKSVQIRPMEQCCCSPRTPCPSSPEELEKLQCDLQDPIVCLADQPHGPPVSSSSKPVPVVPQATIFSHVASDFSLAEPLDHALMTSSTPSLAQETRLPSPQPTISLTSSPAIDLPVQHVVASSSLPQTDLSHTLSPVQSSIPSPTTAAPSVPEKVVAISTPPGETVVNTSSVPDLEAQVSQMEKALSLGSLEPNLAGEMVNRVSKLLHSPLALLAPLAQRLLKVVDAIGLQLNFSSTTISLTSPSLALAVIRVNASNFNTTTFAAQDPANLQVSLEAQAPKNSIGAITLPSSLMSNLPASEVELASRVQFNFFETPALFQDPSLENLSLISYVISSSVTNMTIKNLTRNVTVALKHINPSQDDLTVKCVFWDLNRNGGRGGWSSDGCSVKEKRMNETICTCSHLTSFGILLDLSRTSLPPSQMMALTFITYIGCGLSSIFLSVTLVTYIAFEKIRRDYPSKILIQLCAALLLLNLVFLLDSWIALYNARGFCISVAVFLHYFLLVSFTWMGLEAFHMYLALVKVFNTYIRKYILKFCIVGWGIPAVVVSIVLTISPDNYGIGSYGKFPNGTPDDFCWINSSVVFYITVVGYFCVIFLLNVSMFIVVLVQLCRIKKKKQLGAQRKTSIQDLRSIAGLTFLLGITWGFAFFAWGPVNLTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRMALRRTSKRGSLHFIEQM

SEQ ID NO: 33 - DNA, 4612 bp, Homo sapiens
cgggagcggcgggggcggcgccgaggccgcgggcggggggaggagggccgcagccccggagaggggaaaaggcgggccggacggcgccagcagccagcccgaggacgcgagcggcaggtgtgcacagaggttctccactttgttttctgaactcgcggtcaggatggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgctaaattatctgttgtcagttttgccccctcctccaatggtactccagaggttgaaacaacaagcctcaatgatgttactttaagcttactcccttcaaacgaaacagaaaaaactaaaatcactatagtaaaaaccttcaatgcttcaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgcaatagccgctttggaaagagtaaagattcgaccaatggaacactgctgctgttctgtcaggataccctgcccttcctccccagaagagttgggaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtgattcctttcttctaaaatcaaagcatgatgcttgacagtgtgaaatgtccaattttaccttttacacaatgtgagatgtatgaaaatcaactcattttattctcggcaacatctggagaagcataagctaattaagggcgatgattattattacaagaagaaaccaagacattacaccatggtttttagacatttctgatttggtttcttatctttcattttataagaaggttggttttaaacaatacactaagaatgactcctataaagaaaacaaaaaaaggtagtgaactttcagctaccttttaaagaggctaagttatctttgataacatcatataaagcaactgttgacttcagcctgttggtgagtttagttgtgcatgcctttgttgtatataagctaaattctagtgacccatgtgtcaaaaatcttacttctacatttttttgtatttattttctactgtgtaaatgtattcctttgtagaatcatggttgttttgtctcacgtgataattcagaaaatccttgctcgttccgcaaatcctaaagctccttttggagatgatataggatgtgaaatacagaaacctcagtgaaatcaagaaataatgatcccagccagactgagaaaatgtaagcagacagtgccacagttagctcatacagtgcctttgagcaagttaggaaaagatgcccccactgggcagacacagccctatgggtcatggtttgacaaacagagtgagagaccatattttagccccactcaccctcttgggtgcacgacctgtacagccaaacacagcatccaatatgaatacccatcccctgaccgcatccccagtagtcagattatagaatctgcaccaagatgtttagctttataccttggccacagagagggatgaactgtcatccagaccatgtgtcaggaaaattgtgaacgtagatgaggtacatacactgccgcttctcaaatccccagagcctttaggaacaggagagtagactaggattccttctcttaaaaaggtacatatatatggaaaaaaatcatattgccgttctttaaaaggcaactgcatggtacattgttgattgttatgactggtacactctggcccagccagagctataattgttttttaaatgtgtcttgaagaatgcacagtgacaaggggagtagctattgggaacagggaactgtcctacactgctattgttgctacatgtatcgagccttgattgctcctagttatatacagggtctatcttgcttcctacctacatctgcttgagcagtgcctcaagtacatccttattaggaacatttcaaaccccttttagttaagtctttcactaaggttctcttgcatatatttcaagtgaatgttggatctcagactaaccatagtaataatacacatttctgtgagtgctgacttgtctttgcaatatttcttttctgatttatttaattttcttgtatttatatgttaaaatcaaaaatgttaaaatcaatgaaataaatttgcagttaaga

SEQ ID NO: 34 - protein, 966 aa, Homo sapiens [sequence omitted]

SEQ ID NO: 35 - protein, 1020 aa, Homo sapiens
GSGGGGAEAAGGGRRAAAPERGKGGPDGASSQPEDASGRCAQRFSTLFSELAVRMVFSVRQCGHVGRTEEVLLTFKIFLVIICLHVVLVTSLEEDTDNSSLSPPPAKLSVVSFAPSSNGTPEVETTSLNDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAAIAALERVKIRPMEHCCCSVRIPCPSSPEELGKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSGNASTERNGVSFSVQNGDVCLHDFTGKQHMFNEKEDSCNGKGRMALRRTSKRGSLHFIEQM

SEQ ID NO: 36 - protein, 1033 aa, Homo sapiens
GSGGGGAEAAGGGRRAAAPERGKGGPDGASSQPEDASGRCAQRFSTLFSELAVRMVFSVRQCGHVGRTEEVLLTFKIFLVIICLHVVLVTSLEEDTDNSSLSPPPDVTLSLLPSNETGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAAIAALERVKIRPMEHCCCSVRIPCPSSPEELGKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 37 - protein, 979 aa, Homo sapiens [sequence omitted]

SEQ ID NO: 38 - protein, 1047 aa, Homo sapiens
GSGGGGAEAAGGGRRAAAPERGKGGPDGASSQPEDASGRCAQRFSTLFSELAVRMVFSVRQCGHVGRTEEVLLTFKIFLVIICLHVVLVTSLEEDTDNSSLSPPPDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAAIAALERVKIRPMEHCCCSVRIPCPSSPEELGKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 39 - protein, 993 aa, Homo sapiens [sequence omitted]

SEQ ID NO: 40 - protein, 1057 aa, Homo sapiens
GSGGGGAEAAGGGRRAAAPERGKGGPDGASSQPEDASGRCAQRFSTLFSELAVRMVFSVRQCGHVGRTEEVLLTFKIFLVIICLHVVLVTSLEEDTDNSSLSPPPAKLSVVSFAPSSNGTPEVETTSLNDVTLSLLPSNETGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAAIAALERVKIRPMEHCCCSVRIPCPSSPEELGKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 41 - protein, 1003 aa, Homo sapiens [sequence omitted]

SEQ ID NO: 42 - protein, 1055 aa, Homo sapiens
GSGGGGAEAAGGGRRAAAPERGKGGPDGASSQPEDASGRCAQRFSTLFSELAVRMVFSVRQCGHVGRTEEVLLTFKIFLVIICLHVVLVTSLEEDTDNSSLSPPPEVETTSLNDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAAIAALERVKIRPMEHCCCSVRIPCPSSPEELGKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 43 - protein, 1001 aa, Homo sapiens [sequence omitted]

SEQ ID NO: 44 - protein, 1041 aa, Homo sapiens
GSGGGGAEAAGGGRRAAAPERGKGGPDGASSQPEDASGRCAQRFSTLFSELAVRMVFSVRQCGHVGRTEEVLLTFKIFLVIICLHVVLVTSLEEDTDNSSLSPPPEVETTSLNDVTLSLLPSNETGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAAIAALERVKIRPMEHCCCSVRIPCPSSPEELGKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 45 - protein, 987 aa, Homo sapiens [sequence omitted]

SEQ ID NO: 46 - protein, 1049 aa, Homo sapiens
GSGGGGAEAAGGGRRAAAPERGKGGPDGASSQPEDASGRCAQRFSTLFSELAVRMVFSVRQCGHVGRTEEVLLTFKIFLVIICLHVVLVTSLEEDTDNSSLSPPPAKLSVVSFAPSSNGTPDVTLSLLPSNETGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAAIAALERVKIRPMEHCCCSVRIPCPSSPEELGKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 47 - protein, 995 aa, Homo sapiens [sequence omitted]

SEQ ID NO: 48 - protein, 1071 aa, Homo sapiens
GSGGGGAEAAGGGRRAAAPERGKGGPDGASSQPEDASGRCAQRFSTLFSELAVRMVFSVRQCGHVGRTEEVLLTFKIFLVIICLHVVLVTSLEEDTDNSSLSPPPAKLSVVSFAPSSNGTPEVETTSLNDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAAIAALERVKIRPMEHCCCSVRIPCPSSPEELGKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 49 - protein, 1032 aa, Homo sapiens
QPVTGTRRCTRSFLPISSELPVRMLFSGGQYSPVGRPEEVLLIYKIFLVIICFHVILVTSLKENGNSSLLSPSAESSLVSLIPYSNGTPDAASEVLSTLNKTEKSKITIVKTFNASGVKSQRNICNLSSLCNDSVFFRGEIVFQHDEDHNVTQNQDTANGTFAGVLSLSELKRSELNKTLQTLSETYFIVCATAEAQSTVNCTFTVKLNETMNVCAMMVTFQTVQIRPMEQCCCSPRTPCPSSPEELEKLQCELQDPIVCLADQPHGPPLSSSSKPVVPQATIISHVASDFSLAEPLDHALMTPSTPSLTQESNLPSPQPTIPLASSPATDLPVQSVVVSSLPQTDLSHTLSPVQSSIPSPTTPAPSVPTELVTISTPPGETVVNTSTVSDLEAQVSQMEKALSLGSLEPNLAGEMVNRVSKLLHSPPALLAPLAQRLLKVVDAIGLQLNFSSTTISLTSPSLALAVIRVNASNFNTTTFAAQDPTNLQVSLETPPPENSIGAITLPSSLMNNLPANDVELASRIQFNFFETPALFQDPSLENLTLISYVISSSVTNMTIKNLTRNVTVALKHINPSPDDLTVKCVFWDLGRNGGKGGWSSDGCSVKDKRMNETICTCSHLTSFGILLDLSRTSLPPSQMMALTFITYIGCGLSSIFLSVTLVTYIAFEKIRRDYPSKILIQLCAALLLLNLIFLLDSWIALYNTRGFCIAVAVFLHYFLLVSFTWMGLEAFHMYLALVKVFNTYIRKYILKFCIVGWGIPAVVVSIVLTISPDNYGIGSYGKFPNGTPDDFCWINSNVVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRIALRRTSKRGSLHFIEQM

SEQ ID NO: 50 - protein, 1016 aa, Homo sapiens
QPVTGTRRCTRSFLPISSELPVRMLFSGGQYSPVGRPEEVLLIYKIFLVIICFHVILVTSLKENGNSSLLSPSDAASEVLSTLNKTEKSKITIVKTFNASGVKSQRNICNLSSLCNDSVFFRGEIVFQHDEDHNVTQNQDTANGTFAGVLSLSELKRSELNKTLQTLSETYFIVCATAEAQSTVNCTFTVKLNETMNVCAMMVTFQTVQIRPMEQCCCSPRTPCPSSPEELEKLQCELQDPIVCLADQPHGPPLSSSSKPVVPQATIISHVASDFSLAEPLDHALMTPSTPSLTQESNLPSPQPTIPLASSPATDLPVQSVVVSSLPQTDLSHTLSPVQSSIPSPTTPAPSVPTELVTISTPPGETVVNTSTVSDLEAQVSQMEKALSLGSLEPNLAGEMVNRVSKLLHSPPALLAPLAQRLLKVVDAIGLQLNFSSTTISLTSPSLALAVIRVNASNFNTTTFAAQDPTNLQVSLETPPPENSIGAITLPSSLMNNLPANDVELASRIQFNFFETPALFQDPSLENLTLISYVISSSVTNMTIKNLTRNVTVALKHINPSPDDLTVKCVFWDLGRNGGKGGWSSDGCSVKDKRMNETICTCSHLTSFGILLDLSRTSLPPSQMMALTFITYIGCGLSSIFLSVTLVTYIAFEKIRRDYPSKILIQLCAALLLLNLIFLLDSWIALYNTRGFCIAVAVFLHYFLLVSFTWMGLEAFHMYLALVKVFNTYIRKYILKFCIVGWGIPAVVVSIVLTISPDNYGIGSYGKFPNGTPDDFCWINSNVVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRIALRRTSKRGSLHFIEQM

SEQ ID NO: 51 - protein, 993 aa, Homo sapiens
MLFSGGQYSPVGRPEEVLLIYKIFLVIICFHVILVTSLKENGNSSLLSPSDAASEVLSTLNKTEKSKITIVKTFNASGVKSQRNICNLSSLCNDSVFFRGEIVFQHDEDHNVTQNQDTANGTFAGVLSLSELKRSELNKTLQTLSETYFIVCATAEAQSTVNCTFTVKLNETMNVCAMMVTFQTVQIRPMEQCCCSPRTPCPSSPEELEKLQCELQDPIVCLADQPHGPPLSSSSKPVVPQATIISHVASDFSLAEPLDHALMTPSTPSLTQESNLPSPQPTIPLASSPATDLPVQSVVVSSLPQTDLSHTLSPVQSSIPSPTTPAPSVPTELVTISTPPGETVVNTSTVSDLEAQVSQMEKALSLGSLEPNLAGEMVNRVSKLLHSPPALLAPLAQRLLKVVDAIGLQLNFSSTTISLTSPSLALAVIRVNASNFNTTTFAAQDPTNLQVSLETPPPENSIGAITLPSSLMNNLPANDVELASRIQFNFFETPALFQDPSLENLTLISYVISSSVTNMTIKNLTRNVTVALKHINPSPDDLTVKCVFWDLGRNGGKGGWSSDGCSVKDKRMNETICTCSHLTSFGILLDLSRTSLPPSQMMALTFITYIGCGLSSIFLSVTLVTYIAFEKIRRDYPSKILIQLCAALLLLNLIFLLDSWIALYNTRGFCIAVAVFLHYFLLVSFTWMGLEAFHMYLALVKVFNTYIRKYILKFCIVGWGIPAVVVSIVLTISPDNYGIGSYGKFPNGTPDDFCWINSNVVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRIALRRTSKRGSLHFIEQM

SEQ ID NO: 52 - protein, 1029 aa, Homo sapiens
QPVTGTRRCTRSFLPISSELPVRMLFSGGQYSPVGRPEEVLLIYKIFLVIICFHVILVTSLKENGNSSLLSPSAESSLVSLIPYSNDAASEVLSTLNKTEKSKITIVKTFNASGVKSQRNICNLSSLCNDSVFFRGEIVFQHDEDHNVTQNQDTANGTFAGVLSLSELKRSELNKTLQTLSETYFIVCATAEAQSTVNCTFTVKLNETMNVCAMMVTFQTVQIRPMEQCCCSPRTPCPSSPEELEKLQCELQDPIVCLADQPHGPPLSSSSKPVVPQATIISHVASDFSLAEPLDHALMTPSTPSLTQESNLPSPQPTIPLASSPATDLPVQSVVVSSLPQTDLSHTLSPVQSSIPSPTTPAPSVPTELVTISTPPGETVVNTSTVSDLEAQVSQMEKALSLGSLEPNLAGEMVNRVSKLLHSPPALLAPLAQRLLKVVDAIGLQLNFSSTTISLTSPSLALAVIRVNASNFNTTTFAAQDPTNLQVSLETPPPENSIGAITLPSSLMNNLPANDVELASRIQFNFFETPALFQDPSLENLTLISYVISSSVTNMTIKNLTRNVTVALKHINPSPDDLTVKCVFWDLGRNGGKGGWSSDGCSVKDKRMNETICTCSHLTSFGILLDLSRTSLPPSQMMALTFITYIGCGLSSIFLSVTLVTYIAFEKIRRDYPSKILIQLCAALLLLNLIFLLDSWIALYNTRGFCIAVAVFLHYFLLVSFTWMGLEAFHMYLALVKVFNTYIRKYILKFCIVGWGIPAVVVSIVLTISPDNYGIGSYGKFPNGTPDDFCWINSNVVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRIALRRTSKRGSLHFIEQM

SEQ ID NO: 53 - protein, 1006 aa, Homo sapiens
MLFSGGQYSPVGRPEEVLLIYKIFLVIICFHVILVTSLKENGNSSLLSPSAESSLVSLIPYSNDAASEVLSTLNKTEKSKITIVKTFNASGVKSQRNICNLSSLCNDSVFFRGEIVFQHDEDHNVTQNQDTANGTFAGVLSLSELKRSELNKTLQTLSETYFIVCATAEAQSTVNCTFTVKLNETMNVCAMMVTFQTVQIRPMEQCCCSPRTPCPSSPEELEKLQCELQDPIVCLADQPHGPPLSSSSKPVVPQATIISHVASDFSLAEPLDHALMTPSTPSLTQESNLPSPQPTIPLASSPATDLPVQSVVVSSLPQTDLSHTLSPVQSSIPSPTTPAPSVPTELVTISTPPGETVVNTSTVSDLEAQVSQMEKALSLGSLEPNLAGEMVNRVSKLLHSPPALLAPLAQRLLKVVDAIGLQLNFSSTTISLTSPSLALAVIRVNASNFNTTTFAAQDPTNLQVSLETPPPENSIGAITLPSSLMNNLPANDVELASRIQFNFFETPALFQDPSLENLTLISYVISSSVTNMTIKNLTRNVTVALKHINPSPDDLTVKCVFWDLGRNGGKGGWSSDGCSVKDKRMNETICTCSHLTSFGILLDLSRTSLPPSQMMALTFITYIGCGLSSIFLSVTLVTYIAFEKIRRDYPSKILIQLCAALLLLNLIFLLDSWIALYNTRGFCIAVAVFLHYFLLVSFTWMGLEAFHMYLALVKVFNTYIRKYILKFCIVGWGIPAVVVSIVLTISPDNYGIGSYGKFPNGTPDDFCWINSNVVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRIALRRTSKRGSLHFIEQM

SEQ ID NO: 54 - protein, 1005 aa, Homo sapiens
QPVTGTRRCTRSFLPISSELPVRMLFSGGQYSPVGRPEEVLLIYKIFLVIICFHVILVTSLKDAASEVLSTLNKTEKSKITIVKTFNASGVKSQRNICNLSSLCNDSVFFRGEIVFQHDEDHNVTQNQDTANGTFAGVLSLSELKRSELNKTLQTLSETYFIVCATAEAQSTVNCTFTVKLNETMNVCAMMVTFQTVQIRPMEQCCCSPRTPCPSSPEELEKLQCELQDPIVCLADQPHGPPLSSSSKPVVPQATIISHVASDFSLAEPLDHALMTPSTPSLTQESNLPSPQPTIPLASSPATDLPVQSVVVSSLPQTDLSHTLSPVQSSIPSPTTPAPSVPTELVTISTPPGETVVNTSTVSDLEAQVSQMEKALSLGSLEPNLAGEMVNRVSKLLHSPPALLAPLAQRLLKVVDAIGLQLNFSSTTISLTSPSLALAVIRVNASNFNTTTFAAQDPTNLQVSLETPPPENSIGAITLPSSLMNNLPANDVELASRIQFNFFETPALFQDPSLENLTLISYVISSSVTNMTIKNLTRNVTVALKHINPSPDDLTVKCVFWDLGRNGGKGGWSSDGCSVKDKRMNETICTCSHLTSFGILLDLSRTSLPPSQMMALTFITYIGCGLSSIFLSVTLVTYIAFEKIRRDYPSKILIQLCAALLLLNLIFLLDSWIALYNTRGFCIAVAVFLHYFLLVSFTWMGLEAFHMYLALVKVFNTYIRKYILKFCIVGWGIPAVVVSIVLTISPDNYGIGSYGKFPNGTPDDFCWINSNVVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRIALRRTSKRGSLHFIEQM

SEQ ID NO: 55 - protein, 982 aa, Homo sapiens
MLFSGGQYSPVGRPEEVLLIYKIFLVIICFHVILVTSLKDAASEVLSTLNKTEKSKITIVKTFNASGVKSQRNICNLSSLCNDSVFFRGEIVFQHDEDHNVTQNQDTANGTFAGVLSLSELKRSELNKTLQTLSETYFIVCATAEAQSTVNCTFTVKLNETMNVCAMMVTFQTVQIRPMEQCCCSPRTPCPSSPEELEKLQCELQDPIVCLADQPHGPPLSSSSKPVVPQATIISHVASDFSLAEPLDHALMTPSTPSLTQESNLPSPQPTIPLASSPATDLPVQSVVVSSLPQTDLSHTLSPVQSSIPSPTTPAPSVPTELVTISTPPGETVVNTSTVSDLEAQVSQMEKALSLGSLEPNLAGEMVNRVSKLLHSPPALLAPLAQRLLKVVDAIGLQLNFSSTTISLTSPSLALAVIRVNASNFNTTTFAAQDPTNLQVSLETPPPENSIGAITLPSSLMNNLPANDVELASRIQFNFFETPALFQDPSLENLTLISYVISSSVTNMTIKNLTRNVTVALKHINPSPDDLTVKCVFWDLGRNGGKGGWSSDGCSVKDKRMNETICTCSHLTSFGILLDLSRTSLPPSQMMALTFITYIGCGLSSIFLSVTLVTYIAFEKIRRDYPSKILIQLCAALLLLNLIFLLDSWIALYNTRGFCIAVAVFLHYFLLVSFTWMGLEAFHMYLALVKVFNTYIRKYILKFCIVGWGIPAVVVSIVLTISPDNYGIGSYGKFPNGTPDDFCWINSNVVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRIALRRTSKRGSLHFIEQM

SEQ ID NO: 56 - protein, 1032 aa, Homo sapiens
RTCRCAQSLLPLFSELPVRMLFSGGQYSPVGRPEEVLLIYKIFLVIICFHAILVTSLKENAGNSSLLSPSAESSLVSLVPYSNGTPDAASEVLSTLNRTEKSKITILKTFNASGVKSQRNICNLSSICSDSVFFRGEIVFQHDDHYNVTQNQDIVNSTFAGVLSLSELKRTELNKTLQTLSETYFIVCATAEAQNTLNCTFTVKLNETMNVCAMMVTFKSVQIRPMEQCCCSPRTPCPSSPEELEKLQCDLQDPIVCLADQPHGPPVSSSSKPVPVVPQATIFSHVASDFSLAEPLDHALMTSSTPSLAQETRLPSPQPTISLTSSPAIDLPVQHVVASSSLPQTDLSHTLSPVQSSIPSPTTAAPSVPEKVVAISTPPGETVVNTSSVPDLEAQVSQMEKALSLGSLEPNLAGEMVNRVSKLLHSPLALLAPLAQRLLKVVDAIGLQLNFSSTTISLTSPSLALAVIRVNASNFNTTTFAAQDPANLQVSLEAQAPKNSIGAITLPSSLMSNLPASEVELASRVQFNFFETPALFQDPSLENLSLISYVISSSVTNMTIKNLTRNVTVALKHINPSQDDLTVKCVFWDLNRNGGRGGWSSDGCSVKEKRMNETICTCSHLTSFGILLDLSRTSLPPSQMMALTFITYIGCGLSSIFLSVTLVTYIAFEKIRRDYPSKILIQLCAALLLLNLVFLLDSWIALYNARGFCISVAVFLHYFLLVSFTWMGLEAFHMYLALVKVFNTYIRKYILKFCIVGWGIPAVVVSIVLTISPDNYGIGSYGKFPNGTPDDFCWINSSVVFYITVVGYFCVIFLLNVSMFIVVLVQLCRIKKKKQLGAQRKTSIQDLRSIAGLTFLLGITWGFAFFAWGPVNLTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRMALRRTSKRGSLHFIEQM

SEQ ID NO: 57 - protein, 1016 aa, Homo sapiens
RTCRCAQSLLPLFSELPVRMLFSGGQYSPVGRPEEVLLIYKIFLVIICFHAILVTSLKENAGNSSLLSPSDAASEVLSTLNRTEKSKITILKTFNASGVKSQRNICNLSSICSDSVFFRGEIVFQHDDHYNVTQNQDIVNSTFAGVLSLSELKRTELNKTLQTLSETYFIVCATAEAQNTLNCTFTVKLNETMNVCAMMVTFKSVQIRPMEQCCCSPRTPCPSSPEELEKLQCDLQDPIVCLADQPHGPPVSSSSKPVPVVPQATIFSHVASDFSLAEPLDHALMTSSTPSLAQETRLPSPQPTISLTSSPAIDLPVQHVVASSSLPQTDLSHTLSPVQSSIPSPTTAAPSVPEKVVAISTPPGETVVNTSSVPDLEAQVSQMEKALSLGSLEPNLAGEMVNRVSKLLHSPLALLAPLAQRLLKVVDAIGLQLNFSSTTISLTSPSLALAVIRVNASNFNTTTFAAQDPANLQVSLEAQAPKNSIGAITLPSSLMSNLPASEVELASRVQFNFFETPALFQDPSLENLSLISYVISSSVTNMTIKNLTRNVTVALKHINPSQDDLTVKCVFWDLNRNGGRGGWSSDGCSVKEKRMNETICTCSHLTSFGILLDLSRTSLPPSQMMALTFITYIGCGLSSIFLSVTLVTYIAFEKIRRDYPSKILIQLCAALLLLNLVFLLDSWIALYNARGFCISVAVFLHYFLLVSFTWMGLEAFHMYLALVKVFNTYIRKYILKFCIVGWGIPAVVVSIVLTISPDNYGIGSYGKFPNGTPDDFCWINSSVVFYITVVGYFCVIFLLNVSMFIVVLVQLCRIKKKKQLGAQRKTSIQDLRSIAGLTFLLGITWGFAFFAWGPVNLTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRMALRRTSKRGSLHFIEQM

SEQ ID NO: 58 - protein, 997 aa, Homo sapiens
MLFSGGQYSPVGRPEEVLLIYKIFLVIICFHAILVTSLKENAGNSSLLSPSDAASEVLSTLNRTEKSKITILKTFNASGVKSQRNICNLSSICSDSVFFRGEIVFQHDDHYNVTQNQDIVNSTFAGVLSLSELKRTELNKTLQTLSETYFIVCATAEAQNTLNCTFTVKLNETMNVCAMMVTFKSVQIRPMEQCCCSPRTPCPSSPEELEKLQCDLQDPIVCLADQPHGPPVSSSSKPVPVVPQATIFSHVASDFSLAEPLDHALMTSSTPSLAQETRLPSPQPTISLTSSPAIDLPVQHVVASSSLPQTDLSHTLSPVQSSIPSPTTAAPSVPEKVVAISTPPGETVVNTSSVPDLEAQVSQMEKALSLGSLEPNLAGEMVNRVSKLLHSPLALLAPLAQRLLKVVDAIGLQLNFSSTTISLTSPSLALAVIRVNASNFNTTTFAAQDPANLQVSLEAQAPKNSIGAITLPSSLMSNLPASEVELASRVQFNFFETPALFQDPSLENLSLISYVISSSVTNMTIKNLTRNVTVALKHINPSQDDLTVKCVFWDLNRNGGRGGWSSDGCSVKEKRMNETICTCSHLTSFGILLDLSRTSLPPSQMMALTFITYIGCGLSSIFLSVTLVTYIAFEKIRRDYPSKILIQLCAALLLLNLVFLLDSWIALYNARGFCISVAVFLHYFLLVSFTWMGLEAFHMYLALVKVFNTYIRKYILKFCIVGWGIPAVVVSIVLTISPDNYGIGSYGKFPNGTPDDFCWINSSVVFYITVVGYFCVIFLLNVSMFIVVLVQLCRIKKKKQLGAQRKTSIQDLRSIAGLTFLLGITWGFAFFAWGPVNLTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRMALRRTSKRGSLHFIEQM

SEQ ID NO: 59 - protein, 1004 aa, Homo sapiens
RTCRCAQSLLPLFSELPVRMLFSGGQYSPVGRPEEVLLIYKIFLVIICFHAILVTSLKDAASEVLSTLNRTEKSKITILKTFNASGVKSQRNICNLSSICSDSVFFRGEIVFQHDDHYNVTQNQDIVNSTFAGVLSLSELKRTELNKTLQTLSETYFIVCATAEAQNTLNCTFTVKLNETMNVCAMMVTFKSVQIRPMEQCCCSPRTPCPSSPEELEKLQCDLQDPIVCLADQPHGPPVSSSSKPVPVVPQATIFSHVASDFSLAEPLDHALMTSSTPSLAQETRLPSPQPTISLTSSPAIDLPVQHVVASSSLPQTDLSHTLSPVQSSIPSPTTAAPSVPEKVVAISTPPGETVVNTSSVPDLEAQVSQMEKALSLGSLEPNLAGEMVNRVSKLLHSPLALLAPLAQRLLKVVDAIGLQLNFSSTTISLTSPSLALAVIRVNASNFNTTTFAAQDPANLQVSLEAQAPKNSIGAITLPSSLMSNLPASEVELASRVQFNFFETPALFQDPSLENLSLISYVISSSVTNMTIKNLTRNVTVALKHINPSQDDLTVKCVFWDLNRNGGRGGWSSDGCSVKEKRMNETICTCSHLTSFGILLDLSRTSLPPSQMMALTFITYIGCGLSSIFLSVTLVTYIAFEKIRRDYPSKILIQLCAALLLLNLVFLLDSWIALYNARGFCISVAVFLHYFLLVSFTWMGLEAFHMYLALVKVFNTYIRKYILKFCIVGWGIPAVVVSIVLTISPDNYGIGSYGKFPNGTPDDFCWINSSVVFYITVVGYFCVIFLLNVSMFIVVLVQLCRIKKKKQLGAQRKTSIQDLRSIAGLTFLLGITWGFAFFAWGPVNLTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRMALRRTSKRGSLHFIEQM

SEQ ID NO: 60 - protein, 985 aa, Homo sapiens
MLFSGGQYSPVGRPEEVLLIYKIFLVIICFHAILVTSLKDAASEVLSTLNRTEKSKITILKTFNASGVKSQRNICNLSSICSDSVFFRGEIVFQHDDHYNVTQNQDIVNSTFAGVLSLSELKRTELNKTLQTLSETYFIVCATAEAQNTLNCTFTVKLNETMNVCAMMVTFKSVQIRPMEQCCCSPRTPCPSSPEELEKLQCDLQDPIVCLADQPHGPPVSSSSKPVPVVPQATIFSHVASDFSLAEPLDHALMTSSTPSLAQETRLPSPQPTISLTSSPAIDLPVQHVVASSSLPQTDLSHTLSPVQSSIPSPTTAAPSVPEKVVAISTPPGETVVNTSSVPDLEAQVSQMEKALSLGSLEPNLAGEMVNRVSKLLHSPLALLAPLAQRLLKVVDAIGLQLNFSSTTISLTSPSLALAVIRVNASNFNTTTFAAQDPANLQVSLEAQAPKNSIGAITLPSSLMSNLPASEVELASRVQFNFFETPALFQDPSLENLSLISYVISSSVTNMTIKNLTRNVTVALKHINPSQDDLTVKCVFWDLNRNGGRGGWSSDGCSVKEKRMNETICTCSHLTSFGILLDLSRTSLPPSQMMALTFITYIGCGLSSIFLSVTLVTYIAFEKIRRDYPSKILIQLCAALLLLNLVFLLDSWIALYNARGFCISVAVFLHYFLLVSFTWMGLEAFHMYLALVKVFNTYIRKYILKFCIVGWGIPAVVVSIVLTISPDNYGIGSYGKFPNGTPDDFCWINSSVVFYITVVGYFCVIFLLNVSMFIVVLVQLCRIKKKKQLGAQRKTSIQDLRSIAGLTFLLGITWGFAFFAWGPVNLTFMYLFAIFNTLQGFFIFIFYCAAKENVRKQWRRYLCCGKLRLAENSDWSKTATNGLKKQTVNQGVSSSSNSLQSSCNSTNSTTLLVNSDCSVHASGNGNASTERNGVSFSVQNGDVCLHDLTGKQHMFSDKEDSCNGKSRMALRRTSKRGSLHFIEQM

(19) United States
(12) Patent Application Publication
(10) Pub. No.: US 2006/0069239 A1
(43) Pub. Date: Mar. 30, 2006

(54) Epididymis-specific gene as potential contraceptive target

(76) Inventors: Christiane Kirchhoff, Bordesholm (DE); Heike Obermann-Pless, Luneburg (DE); Annemarie Samalecos, Pinneberg (DE); Caroline Osterhoff, Hamburg (DE); Ulrich Gottwald, Berlin (DE); Reinhard Nubbemeyer, Berlin (DE)

Correspondence Address: MILLEN, WHITE, ZELANO & BRANIGAN, PC, 2200 CLARENDON BLVD, SUITE 1400, ARLINGTON, VA, 22201, US

(21) Appl. No.: 10/532,482

(22) PCT Filed: Oct. 21, 2003
(86) PCT No.: PCT/EP03/11662

§ 371 Date: Apr. 22, 2005

Related U.S. Application Data
(60) Provisional application No. 60/419,979, filed on Oct. 22, 2002.

(51) Int. Cl. A61K 48/00; C07H 21/04; C12P 21/06; A61K 39/395; C07K 14/72; C07K 16/28

(52) U.S. Cl. 530/350; 435/69.1; 435/320.1; 435/325; 530/388.22; 536/23.5; 514/44; 424/143.1

(57) ABSTRACT

Isolated splice variant polypeptides from the family of human epididymis-specific receptors called HE6, and related polypeptides from mouse (ME6) and rat (RE6), are described. Also described are isolated polynucleotides encoding the polypeptides, and fragments and variants of the polypeptides and polynucleotides. The polypeptides are involved in maturation of sperm during their passage through the epididymis, and can be used, e.g., to isolate agents for diagnosing and treating male infertility or for male contraception.

A human epididymis-specific receptor, called HE6, and a cDNA encoding it, have previously been described. See, e.g., Osterhoff et al. (1997), DNA Cell Biol 16, 379-389 and related application U.S. Ser. No. 09/041,746, filed on Mar. 13, 1998, which is incorporated by reference herein in its entirety. The present application discloses the cloning and characterization of nucleic acids encoding rodent (mouse and rat) counterparts of this human receptor and the identification of seven new members (splice variants) of the HE6 (human) family. A number of exons are identified, whose organization is conserved in the three species. Splice variants comprising different combinations of those exons are described for all three species.

The present invention is also directed, e.g., to novel polypeptides whose sequences are predicted from the above nucleic acid sequences, and to isolated fragments and variants of the polypeptides and polynucleotides. The polypeptides of the invention are involved in the maturation of sperm during their passage through the epididymis, and can be used, e.g., to isolate agents for diagnosing and treating male infertility or for male contraception.

In one aspect, the invention relates to isolated HE6-encoding polynucleotides, as represented by SEQ ID NOs: 16 to 22. Each of these polynucleotides is a different splice variant, comprising different combinations of exons selected from the splice region between exons 4-8. These exons are “mini-exons,” each having fewer than 50 nucleotides. FIG. 2 is a diagram showing at least some of the exons and introns of human (HE6), mouse (ME6) and rat (RE6) receptors. As used herein, the term “splice variant refers both to a nucleic acid generated by alternative splicing and to a polynucleotide encoded by it.

The human (HE6) splice variant polynucleotides of the invention are represented by: i) SEQ ID NO: 16 which comprises exons E4 and E7; ii) SEQ ID NO: 17 which comprises exons E4, E7 and E8; iii) SEQ ID NO: 18 which comprises exons E4, E5, b, E6 and E7; iv) SEQ ID NO: 19 (encoding a receptor sometimes referred to herein as human d1) which comprises exons E4, E6, E7 and E8; v) SEQ ID NO: 20 (encoding a receptor sometimes referred to herein as human d2), which comprises exons E4, E6 and E7; vi) SEQ ID NO: 21 (encoding a receptor sometimes referred to herein as human d3), which comprises exons E4, E5, b and E7; and vii) SEQ ID NO: 22 (encoding a receptor sometimes referred to herein as “longest HE6 variant”), which comprises exons E4, E5, b, E6, E7 and E8.

The portions of the above polynucleotides that contain the inventive splice variant regions are represented, respectively, by: i) SEQ ID NO: 1; ii) SEQ ID NO: 2; iii) SEQ ID NO: 3; iv) SEQ ID NO: 4; v) SEQ ID NO: 5; vi) SEQ ID NO: 6; and vii) SEQ ID NO: 7.

The sequences identified by SEQ ID NOS:1-15 are partial sequences derived from SEQ ID NOS:16-29. Specifically, SEQ ID NO:1 is a partial human sequence from SEQ ID NO:16. SEQ ID NO:2 is a partial human sequence from SEQ ID NO: 17. SEQ ID NO:3 is a partial human sequence from SEQ ID NO: 18. SEQ ID NO:4 is a partial human sequence from SEQ ID NO: 19. SEQ ID NO:5 is a partial human sequence from SEQ ID NO: 20. SEQ ID NO:6 is a partial human sequence from SEQ ID NO: 21. SEQ ID NO:7 is a partial human sequence from SEQ ID NO: 22. SEQ ID NO:9 is a partial mouse sequence from SEQ ID NO: 23. SEQ ID NO:10 is a partial mouse sequence from SEQ ID NO: 24. SEQ ID NO:11 is a partial mouse sequence from SEQ ID NO: 25. SEQ ID NO:12 is a partial mouse sequence from SEQ ID NO: 26. SEQ ID NO:13 is a partial rat sequence from SEQ ID NO: 27. SEQ ID NO:14 is a partial rat sequence from SEQ ID NO: 28. SEQ ID NO:15 is a partial rat sequence from SEQ ID NO: 29.

A polypeptide encoded by the longest of the HE6 splice variant polynucleotides (SEQ ID NO: 22) is represented by SEQ ID NO: 30. A skilled worker can readily determine the predicted polypeptide sequences encoded by the remaining HE6 splice variant polynucleotides.

The invention also relates to HE6 polypeptides that are translated from the first in phase AUG in the cDNAs (at position 164-167 of, e.g., SEQ ID NO: 22), i.e., a polypeptide which initiates at amino acid 55 of the polypeptide represented by SEQ ID NO: 22 and to comparable polypeptides encoded by the other HE6 splice variants of the invention. A skilled worker can readily determine the sequences of the polypeptides encoded by the remaining HE6 splice variants which initiate at the equivalent AUG.

In another aspect, the invention relates to a 91 nt oligonucleotide sequence at the 5′ end of the HE6 polynucleotide sequences represented by SEQ ID NOs: 16 to 22. This 91 nt sequence is represented by SEQ ID NO: 8. The invention also encompasses an isolated polynucleotide wherein this 91 nt sequence is covalently bound, in phase, to the 5′ end of the polynucleotide encoding the previously reported HE6 polypeptide (Genbank accession number X81892), and to a polypeptide encoded by this polynucleotide (i.e., an isolated polypeptide having the 30 amino acid sequence at its N-terminus).

In another aspect, the invention relates to isolated mouse equivalents of the HE6 polypeptides and polynucleotides. The mouse epididymis-specific receptor-6 (ME6)-encoding polynucleotides are represented by SEQ ID NOs: 23, 24, 25, and 26. As is the case for the human epididymis-specific receptor polynucleotides, each of the mouse polynucleotides is a different splice variant, comprising different arrangements of exons selected from the splice region between exons 4-8. The ME6 splice variants of the invention are:
i) SEQ ID NO: 23 (encoding a receptor sometimes referred to herein as “the longest ME6 variant”), which comprises exons E4, E5, b, E7 and E8;
ii) SEQ ID NO: 24 (encoding a receptor sometimes referred to herein as mouse d1), which comprises exons E4, E7 and E8;
iii) SEQ ID NO: 25 (encoding a receptor sometimes referred to herein as mouse d2), which comprises exons E4, E5, E7 and E8; and
iv) SEQ ID NO: 26 (encoding a receptor sometimes referred to herein as mouse d3), which comprises exons E7 and E8.

The portions of the above polynucleotides which contain the unique splice variant regions are represented, respectively, by:
i) SEQ ID NO: 9;
ii) SEQ ID NO:10;
iii) SEQ ID NO: 11; and
iv) SEQ ID NO: 12. A polypeptide encoded by the longest of the ME6 splice variants polynucleotides (SEQ ID NO: 23) is represented by SEQ ID NO: 31.
   A skilled worker can readily determine the sequences of the comparable polypeptides encoded by the other ME6 polynucleotide splice variant nucleic acids.

The invention also relates to polypeptides encoded by each of the ME6 splice variant nucleic acids, which begin translation at the first in phase AUG of those sequences (located at position 60-62 of the cDNAs). For example, such a protein encoded by the longest ME6 splice variant polynucleotide is represented by a polypeptide beginning at amino acid position 1 of SEQ ID NO: 31.

In another aspect, the invention relates to isolated rat equivalents of the HE6 polypeptides and polynucleotides. The rat epididymis-specific receptor-6 (RE6)-encoding polynucleotides are represented by SEQ ID NOs: 27, 28 and 29. As is the case for the human epididymis-specific receptor polynucleotides, each of the rat polynucleotides is a different splice variant, comprising different arrangements of exons selected from the splice region between exons 4-8. The RE6 splice variants of the invention are:
i) SEQ ID NO: 27 (encoding a receptor sometimes referred to herein as “the longest RE6 variant”), which comprises exons E4, E5, b, E7 and E8;
ii) SEQ ID NO: 28 (encoding a receptor sometimes referred to herein as rat d1), which comprises exons E4, E7 and E8; and
iii) SEQ ID NO: 29 (encoding a receptor sometimes referred to herein as rat d2), which comprises exons E7 and E8.

The portions of the above polynucleotides which contain the unique splice variant regions are represented, respectively, by:
i) SEQ ID NO: 13;
ii) SEQ ID NO:14; and
iii) SEQ ID NO: 15.

A polynucleotide encoded by the longest of the RE6 splice variants (SEQ ID NO: 27) is represented by SEQ ID NO: 32. A skilled worker can readily determine the sequences of the comparable polypeptides encoded by the other RE6 polynucleotide splice variant nucleic acids.

The invention also relates to polypeptides encoded by each of the RE6 splice variant nucleic acids, which begin translation at the first in phase AUG of those sequences (located at position 60-62 of the cDNAs). For example, such a protein encoded by the longest RE6 splice variant polynucleotide is represented by a polypepitde beginning at amino acid 1 of SEQ ID NO: 32.

In one aspect, the present invention relates to an isolated human epididymis-specific receptor protein-6 (HE6) polypeptide comprising amino acids encoded by SEQ ID NO: 1, 2, 3, 4, 5, 6, or 7 or a functional variant or fragment thereof.

In another aspect, the invention relates to an isolated polypeptide comprising an amino acid sequence which has at least 65%, 70-75%, 80-85%, 90-95%, or 97-99% sequence identity to any of the HE6 polypeptides encoded by any of the sequences shown in SEQ ID NOS:1-7.

In another aspect, the invention relates to a polypeptide comprising amino acid sequences encoded by SEQ ID NO: 1, 2, 3, 4, 5, 6, or 7 and further comprises a heterologous sequence.

In another aspect, the invention relates to an isolated polypeptide encoded by SEQ ID NOS: 16-22, starting with AUG at nucleotide position 164-166 and terminating at the stop codon at positions 3101-3103, 3143-3145, 3173-3175, 3167-3169, 3125-3127, or 3149-3151.

In another aspect, the invention relates to an isolated polypeptide encoded by SEQ ID NOS: 16-22, starting at the codon at position 2-4 and terminating at the stop codon at positions 3101-3103, 3143-3145, 3173-3175, 3167-3169, 3125-3127, or 3149-3151.

In another aspect, the invention relates to an isolated polypeptide comprising from N-terminus to G-terminus the polypeptide sequence represented by amino acids 1 through 54 encoded by the nucleotide sequence shown in SEQ ID NO:22 covalently bound to the polypeptide X81892.

In another aspect, the invention relates to an isolated mouse epididymis-specific receptor protein-6 (ME6) comprising amino acid sequences encoded by SEQ ID NO: 9, 10, 11, or 12 or functional fragments or variants thereof.

In another aspect, the invention relates to an isolated polypeptide comprising an amino acid sequence which has at least 65%, 70-75%, 80-85%, 90-95%, or 97-99% sequence identity to any of the ME6 polypeptides encoded by sequences shown in SEQ ID NOS:9-12.

In another aspect, the invention relates to a polypeptide comprising amino acids 1 to 1009 of SEQ ID NO:31.

In another aspect, the invention relates to a polypeptide comprising amino acid sequences encoded by SEQ ID NO: 9, 10, 11, or 12 and further comprises a heterologous sequence.

In another aspect, the invention relates to an isolated polypeptide comprising amino acid sequences encoded by SEQ ID NOS:23-26, starting with AUG at nucleotide position 72-74 and terminating at stop codons 3099-3101, 3051-3053, 3090-3092, and 3018-3020 for SEQ ID NOS:23-26, respectively.

In another aspect, the invention relates to an isolated polypeptide which comprises the sequence beginning with amino acid 1 of the sequence represented by SEQ ID NO:31.

In another aspect, the invention relates to an isolated rat epididymis-specific receptor protein-6 (ME6) comprising amino acid sequences encoded by SEQ ID NO: 13, 14, or 15 or functional fragments or variants thereof.

In another aspect, the invention relates to an isolated polypeptide comprising an amino acid sequence which has at least 65%, 70-75%, 80-85%, 90-95%, or 97-99% sequence identity to any of the RE6 polypeptides encoded by sequences encoded by SEQ ID NOS:13-15.

In another aspect, the invention relates to an isolated polypeptide comprising the amino acid sequences encoded by SEQ ID NOS: 27, 28, or 29, starting with the AUG at nucleotide positions at 60-62 and terminating at the stop codon at positions 3099-3101, 3051-3053, and 3015-3017 for SEQ ID NOS:27-29, respectively.

In another aspect, this invention relates to a recombinant construct comprising the polynucleotides of SEQ ID NO:1, 2, 3, 4, 5, 6, or 7 operatively linked to a regulatory sequence.

In another aspect, this invention relates to a monoclonal or polyclonal antibody or an antigen-specific fragment specific for a human HE6 polypeptide comprising amino acid sequences encoded by SEQ ID NO: 1, 2, 3, 4, 5, 6, or 7.

In another aspect, this invention relates to a monoclonal or polyclonal antibody or an antigen-specific fragment specific for a mouse ME6 polypeptide comprising amino acid sequences encoded by SEQ ID NO: 9, 10, 11, or 12.

In another aspect, this invention relates to a monoclonal or polyclonal antibody or antigen-specific fragment specific for a rat RE6 polypeptide comprising amino acid sequences encoded by SEQ ID NO: 27, 28, or 29.

In another aspect, this invention relates to a pharmaceutical composition comprising an antagonist or inhibitor of a polypeptide comprising amino acid sequences encoded by SEQ ID NO: 1, 2, 3, 4, 5, 6, 7, 8, 16, 17, 18, 19, 20, 21, or 22.

In another aspect, the invention relates to a pharmaceutical composition comprising an antisense oligonucleotide which can bind with any of the nucleotide sequences shown in SEQ ID NO: 1, 2, 3, 4, 5, 6, 7, 8, 16, 17, 18, 19, 20, 21, or 22.

In another aspect, this invention relates to a composition for treating a male reproductive disorder comprising administering an effective amount of a polypeptide comprising amino acid sequences encoded by SEQ ID NO: 1, 2, 3, 4, 5, 6, 7, 8, 16, 17, 18, 19, 20, 21, or 22.

In another aspect, this invention relates to a composition for diagnosing a male reproductive disorder comprising administering an effective amount of a polypeptide comprising amino acid sequences encoded by SEQ ID NO: 1, 2, 3, 4, 5, 6, 7, 12, 13, 14, 15, 27, 28, or 29.

In another aspect, this invention relates to a method for isolating an agent for modulating expression activity of an epididymis-specific receptor comprising incubating said epidiymis-specific receptor comprising an amino acid sequence encoded by SEQ ID NO: 1, 2, 3, 4, 5, 6, 7, 12, 13, 14, 15, 27, 28, or 29 with a putative agent, and measuring the amount of activity of said receptor or polynucleotide.

In another aspect, the invention relates to a method for diagnosing infertility in a male mammal, which is associated with under-expression or over-expression of a polynucleotide comprising a sequence of SEQ ID NO: 1, 2, 3, 4, 5, 6, 7, 8, 16, 17, 18, 19, 20, 21, or 22 comprising contacting a tissue, cell, or polynucleotide from said male with a probe that is specific for said sequence and determining the amount of nucleic acid that hybridizes to the probe wherein said cell or tissue is from a biopsy sample or thin section from the epididymis of said male mammal.

Another aspect of the invention relates to a method for diagnosing infertility in a male mammal comprising measuring antibodies from said male specific for a polypeptide comprising a sequence of SEQ ID NO: 1, 2, 3, 4, 5, 6, 7, 8, 16, 17, 18, 19, 20, 21 or 22.

In another aspect the invention relates to a method for treating infertility in a male mammal comprising administering to said mammal an agonist or antagonist of an epididymis-specific receptor comprising administering an effective amount of a polypeptide comprising amino acid sequence encoded by SEQ ID NO: 1, 2, 3, 4, 5, 6, 7, 8, 16, 17, 18, 19, 20, 21 or 22.

In another aspect, the invention relates to a method for contraception in a male mammal comprising administering to said mammal an antagonist of an epididymis-specific receptor comprising a polypeptide encoded by SEQ ID NO: 1, 2, 3, 4, 5, 6, 7, 8, 16, 17, 18, 19, 20, 21 or 22.

In another aspect, the invention relates to a splice variant which lacks exon 24 of the human HE6 gene (SEQ ID NO: 33). The polypeptide which is translated from the nucleotide sequence shown in SEQ ID NO:33 includes one sequence which includes the upstream open reading frame (SEQ ID NO:34) and the sequence which excludes the upstream open reading frame (SEQ ID NO:35).

In another aspect the invention relates to a recombinant construct comprising the polynucleotide of SEQ ID NO:33 operatively linked to a regulatory sequence.

In another aspect the invention relates to a monoclonal or polyclonal antibody or an antigen-specific fragment specific for human HE6 polypeptide comprising amino acid sequences encoded by SEQ ID NO:33.

In another aspect the invention relates to a pharmaceutical composition comprising an antagonist or inhibitor of a polypeptide comprising amino acid sequences encoded by SEQ ID NOS: 34 or 35.

In another aspect the invention relates to a composition for treating a male reproductive disorder comprising administering an effective amount of a polypeptide comprising an amino acid sequence encoded by SEQ ID NO:33.

In another aspect the invention relates to a composition for diagnosing a male reproductive disorder comprising administering an effective amount of a polypeptide comprising amino acid sequences encoded by SEQ ID NO:33.

In another aspect the invention relates to proteins which are translated from the various splice variants (SEQ ID NOS:16-29) including the sequence which includes the upstream open reading frame (+up) and an alternate sequence which does not include the upstream open reading frame (−up). More specifically, SEQ ID NO:16 is translated into two polypeptide sequences SEQ ID NOS: 36 and 37 wherein the upstream open reading frame is included (SEQ ID NO:36) or excluded (SEQ ID NO:37). SEQ ID NO:17 is translated into two polypeptide sequences SEQ ID NOS: 38 and 39 wherein the upstream open reading frame is included (SEQ ID NO:38) or excluded (SEQ ID NO:39). SEQ ID NO:18 is translated into two polypeptide sequences SEQ ID NOS: 40 and 41 wherein the upstream open reading frame is included (SEQ ID NO:40) or excluded (SEQ ID NO:41). SEQ ID NO:19 is translated into two polypeptide sequences SEQ ID NOS: 42 and 43 wherein the upstream open reading frame is included (SEQ ID NO:42) or excluded (SEQ ID NO:43). SEQ ID NO:20 is translated into two polypeptide sequences SEQ ID NOS: 44 and 45 wherein the upstream open reading frame is included (SEQ ID NO:44) or excluded (SEQ ID NO:45). SEQ ID NO:21 is translated into two polypeptide sequences SEQ ID NOS: 46 and 47 wherein the upstream open reading frame is included (SEQ ID NO:46) or excluded (SEQ ID NO:47). SEQ ID NO:22 is translated into two polypeptide sequences SEQ ID NOS: 48 and 49 wherein the upstream open reading frame is included (SEQ ID NO:48) or excluded (SEQ ID NO:49). SEQ ID NO:23 is translated into a polypeptide sequence SEQ ID NO:49 wherein the upstream open reading frame is included. SEQ ID NO:24 is translated into two polypeptide sequences SEQ ID NOS: 50 and 51 wherein the upstream open reading frame is included (SEQ ID NO:50) or excluded (SEQ ID NO:51). SEQ ID NO:25 is translated into two polypeptide sequences SEQ ID NOS: 52 and 53 wherein the upstream open reading frame is included (SEQ ID NO:52) or excluded (SEQ ID NO:53). SEQ ID NO:26 is translated into two polypeptide sequences SEQ ID NOS: 54 and 55 wherein the upstream open reading frame is included (SEQ ID NO:54) or excluded (SEQ ID NO:55). SEQ ID NO:27 is translated into a polypeptide sequences SEQ ID NO: 56 wherein the upstream open reading frame is included. SEQ ID NO:28 is translated into two polypeptide sequences SEQ ID NOS: 57 and 58 wherein the upstream open reading frame is included (SEQ ID NO:57) or excluded (SEQ ID NO:58). SEQ ID NO:29 is translated into two polypeptide sequences SEQ ID NOS: 59 and 60 wherein the upstream open reading frame is included (SEQ ID NO:59) or excluded (SEQ ID NO:60).

Clones of the human, rat and mouse epididymis-specific HE6-type proteins were obtained and sequenced, using conventional procedures, e.g. as described in Examples 1 and 2. By comparing various cloned cDNAs to one another and to known genomic clones, mini-exons (each having fewer than 50 nts) were identified whose organization is conserved in all three species. Splice variants were identified, which affect the amino acid sequences of the N-terminal-most part of the receptors. FIG. 2 diagramatically shows the map positions of newly-identified exons 4-8, and the structures of the splice variants. Seven new splice variants are shown for the human family (HE6), four for the mouse family (ME6) (plus one identified in an EST library—Genbank accession no: B1155218), and three for the rat family (RE6).

Characterization of the proteins is shown, e.g., in Examples 3 and 4. The proteins, which are tissue-specific seven-membrane receptors of the epididymis, are shown to have a two-subunit structure: an approximately 180 kD hydrophilic ectosubunit, which is highly glycosylated, and a <40 kD hydrophobic endosubunit. Example 5 shows immunological studies, which reveal that both subunits are associated with apical membrane of efferent ductule and proximal epididymal duct epithelia.

Using conventional procedures, it has been shown that: the receptor proteins according to the invention are present in the epididymis in a high abundance; they are highly conserved among mammalian species; and the mRNAs are localized in epithelial cells that line the ductus epididymis.

The polypeptides of the present invention are preferably provided in an isolated form, and may be purified, e.g, to homogeneity. The term “isolated,” when referring, e.g., to a polypeptide or polynucleotide, means that the material is removed from its original environment (e.g., the natural environment if it is naturally occurring), and isolated or separated from at least one other component with which it is naturally associated. For example, a naturally-occurring polypeptide present in its natural living host is not isolated, but the same polypeptide, separated from some or all of the coexisting materials in the natural system, is isolated. Such polypeptides could be part of a composition, and still be isolated in that such composition is not part of its natural environment.

As used herein, the terms “polypeptide” and “protein” are interchangeable.

The invention also relates to functional fragments or functional variants of polypeptides of the invention. By “functional” fragments or variants is meant herein that the fragment or variant exhibits a biological activity and/or immunogenicity of the polypeptide from which it is derived. Discussions herein of fragments or variants of the invention are directed to “functional” fragments or variants. According to the invention, the term “proteins or polypeptides having the same biological activity and/or immunogenicity” designates molecules which have a) the same epididymal specificity and b) the same ligand-binding capacity as the proteins and polypeptides identified according to the invention.

According to the invention, the term “ligand” includes both antibodies against any desired epitopes included in the proteins or polypeptides according to the invention, and other chemical substances or molecules which are capable of binding to one or more of the domains present in the proteins or polypeptides according to the invention. In a preferred embodiment, a ligand interacts specifically (preferentially) with sequences from the extracellular, N-terminal, portion of a receptor protein of the invention that are encoded by a novel splice variant region as noted herein. Alternatively, a ligand may bind to sequences whose secondary and/or tertiary structure is altered by interaction with a novel splice variant peptide sequence of the invention.

In one embodiment, the present invention provides isolated or purified peptides: human N2, mouse N2, human A or mouse A23, as described in Example 1D.

Polypeptide fragments of the invention may be of any size that is compatible with the invention. They may range in size from the smallest specific epitope (e.g., about 6 amino acids) to a nearly full-length gene product (e.g., a single amino acid shorter than a full-length polypeptide).

Fragments of the polypeptides of the present invention may be employed, e.g., for producing the corresponding full-length polypeptide by peptide synthesis, e.g., as intermediates for producing the full-length polypeptides; for inducing the production of antibodies or antigen-binding fragments; as “query sequences” for the probing of public databases, or the like.

A variant of a polypeptide of the invention may be, e.g.,
(i) one in which one or more of the amino acid residues are substituted with a conserved or non-conserved amino acid residue (preferably a conserved amino acid residue) and such substituted amino acid residue may or may not be one encoded by the genetic code, or
(ii) one in which one or more of the amino acid residues includes a substituent group, or
   (iii) one in which the polypeptide is fused with another compound, such as a compound to increase the half-life of the polypeptide (for example, polyethylene glycol), or
(iv) one in which additional amino acids are fused to the polypeptide, such as a leader or secretory sequence or a sequence which is employed for purification of the polypeptide, commonly for the purpose of creating a genetically engineered form of the protein that is susceptible to secretion from a cell, such as a transformed cell. The additional amino acids may be from a heterologous source, or may be endogenous to the natural gene.

Variant polypeptides belonging to type (i) above include, e.g., analogs, muteins and derivatives. A variant polypeptide can differ in amino acid sequence by, e.g., one or more additions, substitutions, deletions, insertions, inversions, fusions, and truncations or a combination of any of these. Naturally occurring allelic variants are included.

Variant polypeptides belonging to type (ii) above include, e.g., modified polypeptides. Known polypeptide modifications include, but are not limited to, glycosylation, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formatin, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in many basic texts, such as Proteins-Structure and Molecular Properties, 2nd ed., T.E. Creighton, W.H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (1990) Meth. Enzymol. 182:626-646 and Rattan et al. (1992) Ann. N.Y. Acad. Sci. 663:48-62.

Variant polypeptides belonging to type (iii) are well-known in the art and include, e.g., PEGylation or other chemical modifications.

Variants polypeptides belonging to type (iv) above include, e.g., preproteins or proproteins which can be activated by cleavage of the proprotein portion to produce an active mature polypeptide. Variants include a variety of hybrid, chimeric or fusion polypeptides. Typical examples of such variants are discussed elsewhere herein.

Many other types of variants are known to those of skill in the art. For example, as is well known, polypeptides are not always entirely linear. For instance, polypeptides may be branched as a result of ubiquitination, and they may be circular, with or without branching, generally as a result of post-translation events, including natural processing events and events brought about by human manipulation which do not occur naturally. Circular, branched and branched circular polypeptides may be synthesized by non-translational natural processes and by synthetic methods.

Modifications or variations can occur anywhere in a polypeptide, including the peptide backbone, the amino acid side-chains and the amino or carboxyl termini. The same type of modification may be present in the same or varying degree at several sites in a given polypeptide. Also, a given polypeptide may contain more than one type of modification. Blockage of the amino or carboxyl group in a polypeptide, or both, by a covalent modification, is common in naturally-occurring and synthetic polypeptides. For instance, the aminoterminal residue of polypeptides made in E. coli, prior to proteolytic processing, is often N-formylmethionine. The modifications can be a function of how the protein is made. For recombinant polypeptides, for example, the modifications are determined by the host cell posttranslational modification capacity and the modification signals in the polypeptide amino acid sequence. Accordingly, when glycosylation is desired, a polypeptide can be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

The polypeptides of the invention also include polypeptides which have varying degrees of sequence homology (identity) to polypeptides as described above.

In the case of human (HE6) polypeptides, such homologues contain a sequence that is substantially homologous, or which shows substantial sequence homology (identity), to a novel peptide encoded by sequences of exons 4-8. Homologues comprising HE6 X81892 are excluded. Thus, polypeptides, and fragments thereof, of the human polypeptides may contain sequences from the unique splice variant region which show at least about 65% sequence homology (identity) to the comparable region of a human splice variant of the invention, preferably about 70-75% or 80-85% sequence homology (identity) thereto, and most preferably about 90-95% or 97-99% sequence homology (identity) thereto. The invention also encompasses polypeptides having a lower degree of sequence identity, but having sufficient similarity so as to perform one or more of the functions or activities exhibited by the receptors of the invention.

Comparable homologues of the mouse and rat polypeptides are also contemplated by the invention. For the rodent homologues, the sequence homology (identity) may be to any portion of the polypeptide. Mouse homologues comprising ME6 BI155218 are excluded.

In accordance with the present invention, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:
Percent Identity=100[1−(C/R)]
wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the amino acid sequences herein having 91 amino acid residues, at least 30, preferably at least 35, more preferably at least 45, even more preferably at least 55, and even more preferably at least 65, 70, 80 and 90 amino acid residues are aligned).

The description herein for percent identity or percent homology is intended to apply equally to nucleotide or amino acid sequences

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

A preferred, non-limiting example of such a mathematical algorithm is described in Karlin et al. (1993) Proc. Natl. Acad. Sci. USA 90:5873-5877. Such an algorithm is incorporated into the NBLAST and XBLAST programs (version 2.0) as described in Altschul et al. (1997) Nucleic Acids Res. 25:3389-3402. When utilizing BLAST and Gapped BLAST programs, the default parameters of the respective programs (e.g., NBLASST) can be used. In one embodiment, parameters for sequence comparison can be set at score=100, wordlength-12, or can be varied (e.g., W=5 or W=20).

In a preferred embodiment, the percent identity between two amino acid sequences is determined using the Needleman et al. (1970) (J. Mol. Biol. 48:444-453) algorithm which has been incorporated into the GAP program in the GCG software package using either a BLOSUM 62 matrix or a PAM250 matrix, and a gap weight of 16, 14, 12, 10, 8, 6, or 4 and a length weight of 1, 2, 3, 4, 5 or 6. In yet another preferred embodiment, the percent identity between two nucleotide sequences is determined using the GAP program I the GCG software package (Devereux et al. (1984) Nucleic Acids Res. 12 (1):387) using a NWSgapdna. CMP matrix and a gap weight of 40, 50, 60, 70, or 80 and a length weight of 1, 2, 3, 4, 5 or 6.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis et al. (1994) Comput. Appl. Biosci. 10:3-5; and FASTA described in Pearson et al. (1988) PNAS 85:2444-8.

In accordance with the present invention, the term substantially homologous,” when referring to a protein sequence, means that the amino acid sequences are at least about 90-95% or 97-99% or more identical. A substantially homologous amino acid sequence of the invention can be encoded by a nucleic acid sequence hybridizing to the nucleic acid sequence, or portion thereof, encoding it under conditions of high stringency. Conditions of “high stringency,” as used herein, means, for example, incubating a blot overnight (e.g., at least 12 hours) with a polynucleotide probe in a hybridization solution containing, e.g., about 5×SSC, 0.5% SDS, 100 μg/ml denatured salmon sperm DNA and 50% formamide, at 42° C. Blots can be washed at high stringency conditions that allow, e.g., for less than 5% bp mismatch (e.g., wash twice in 0.1×SSC and 0.1% SDS for 30 min at 65° C.), thereby selecting sequences having, e.g., 95% or greater sequence identity. Other non-limiting examples of high stringency conditions include a final wash at 65° C. in aqueous buffer containing 30 mM NaC1 and 0.5% SDS. Another example of high stringent conditions is hybridization in 7% SDS, 0.5 M NaPO4, pH 7, 1 mM EDTA at 50° C., e.g., overnight, followed by one or more washes with a 1% SDS solution at 42° C. Whereas high stringency washes can allow for less than 5% mismatch, reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

The invention also relates to polynucleotides that encode the polypeptides, and variants or fragments thereof, of the invention. For the human HE6 splice variants, a polynucleotide of the invention comprises at least a portion of an inventive combination of exons 3-8. For the mouse and rat splice variant polynucleotides, the polynucleotides can encode any portion of the ME6 or RE6 polypeptides of the invention, provided that the polynucleotide sequence is not the mouse EST sequence, BI155218.

Nucleotide sequences which code for the receptor proteins and variants and fragments according to the invention can be transferred into prokaryotic or eukaryotic host cells by conventional methods via suitable vectors and expressed there as protein. The present invention includes vectors and host cells containing these nucleotide sequences as well as host cells transformed by same, and the recombinantly expressed proteins and fragments.

All nucleic acid sequences which, after transformation of suitable prokaryotic and/or eukaryotic host cells, ensure the production of nucleic acids (including fragments) for use as diagnostics, and/or the expression of proteins or polypeptides as noted above which have one or more of the biological and/or immunogenic properties of the receptor proteins according to the invention, are suitable and are included according to the invention. These sequences, in single- or double-stranded form, include, any of the nucleic acid sequences noted above. Sequences that are complementary to any polynucleotide or fragment described herein are also included.

As used herein, the terms “nucleic acid” and “polynucleotide” are interchangeable.

A polynucleotide of the present invention may be a recombinant polynucleotide, a natural polynucleotide, or a synthetic or semi-synthetic polynucleotide, or combinations thereof.

Polynucleotides of the invention may be RNA, PNA, or DNA, e.g., cDNA, and synthetic or semi-synthetic DNA, or combinations thereof. They may code without interruption for a polypeptide of the invention. A polynucleotide which “codes without interruption” refers to a polynucleotide having a continuous open reading frame (“ORF”) as compared to an ORF which is interrupted by introns or other noncoding sequences. The DNA may be triplex, double-stranded or single-stranded, and if single stranded, may be the coding strand or non-coding (anti-sense) strand. It can comprise hairpins or other secondary structures.

The RNA includes oligomers (including those having sense or antisense strands), mRNAs (e.g., having the alternative splices of the epididymis-specific genes of the invention), polyadenylated RNA, total RNA, single strand or double strand RNA, or the like. DNA/RNA duplexes are also encompassed by the invention.

Polynucleotides of the invention may contain one or more modified nucleotides, e.g., nucleotides that include a substituent group. Such modifications include, e.g., the attachment of detectable markers (avidin, biotin, radioactive elements, fluorescent tags and dyes, energy transfer labels, energy-emitting labels, binding partners, etc.) or moieties which improve expression, uptake, cataloging, tagging, hybridization, detection, and/or stability. The polynucleotides can also be attached to solid supports, e.g., nitrocellulose, magnetic or paramagnetic microspheres (e.g., as described in U.S. Pat. No. 5,411,863; U.S. Pat. No. 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967; 5,476,925; 5,478,893.

Polynucleotides of the invention may be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, etc.

Any of a variety of variant nucleic acids are encompassed by the invention. The term “variant” sequence includes all sequences which are derived from the same or a homologous or a similar gene and code for a receptor protein in the context of the invention, or can be used for the preparation of probes. Naturally occurring polynucleotide variants, e.g., allelic variants, SNPs, or the like, are included.

Variants include, for example, sequences which show deviations on the basis of degeneracy of the genetic code. For example, a variant sequence may encode an ESRP (epididymis-specific receptor protein) of the invention, but, instead of having the sequence of the natural nucleic acid, may have a sequence that corresponds to the codon usage of the host organism into which the nucleic acid construct is introduced.

Other suitable modifications are nucleotide substitutions which give rise to a different protein amino acid sequence and therefore, possibly, a different protein structure without, however, impairing the properties (e.g., a biological activity, or immunogenicity) of the native polynucleotide. Suitable variants include, e.g., insertions of one or more nucleotides, transitions, transversions, inversions, addition of one or more nucleotides at either end of the sequence, or deletion of one or more nucleotides at either end or within the sequence.

Polynucleotides of the invention may also be fused with another compound, such as a compound to increase the half-life of the polynucleotide, or may comprise additional nucleotides that are covalently bound to the polynucleotide, such a sequences encoding a leader or secretory sequence or a sequence which is employed for purification of the polypeptide. The additional nucleotides may be from a heterologous source, or may be endogenous to the natural gene. For example, a polynucleotide of the invention may comprise a coding sequence and one or more additional non-naturally occurring or heterologous coding sequences (e.g., sequences coding for leader, signal, secretory, targeting, enzymatic, fluorescent, antibiotic resistance, and other functional or diagnostic peptides); or a coding sequence and non-coding sequences, e.g., untranslated sequences at either a 5′ or 3′ end, or dispersed in the coding sequence, e.g., introns.

More specifically, the present invention includes polynucleotides wherein the coding sequence for the polypeptide is fused in the same reading frame to a polynucleotide sequence (e.g., a heterologous sequence), e.g. one which aids in expression and secretion of a polypeptide from a host cell, such as a leader sequence which functions as a secretory sequence for controlling transport of a polypeptide from the cell and/or a transmembrane anchor which facilitates attachment of the polypeptide to a cellular membrane.

Polynucleotides of the present invention may also have a coding sequence fused in frame to a marker sequence that allows for identification and/or purification of the polypeptide of the present invention. The marker sequence may be, e.g., a hexa-histidine tag (e.g., as supplied by a pQE-9 is vector) to provide for purification of the mature polypeptide fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein (Wilson, I., et al., Cell, 37:767 (1984)).

The polynucleotides of the invention also include polynucleotides which have varying degrees of sequence homology (identity) to the polynucleotides described above.

In the case of human (HE6) polynucleotides, such homologues contain a sequence that is substantially homologous, or which shows substantial sequence homology (identity), to inventive combinations of exons 4-8.

Homologues comprising HE6 X81892 are excluded. Thus, polynucleotides, and fragments thereof, of the human polynucleotides may contain sequences from the unique splice variant region which show at least about 65% sequence homology (identity) to the comparable region of a human splice variant of the invention, preferably about 70-75% or 80-85% sequence homology (identity) thereto, and most preferably about 90-95% or 97-99% sequence homology (identity) thereto. The invention also encompasses polynucleotides having a lower degree of sequence identity, but having sufficient similarity so as to perform one or more of the functions or activities exhibited by the receptors of the invention.

Comparable homologues of the mouse and rat polynucleotides are also contemplated by the invention. For the rodent homologes, the sequence homology (identity) may be to any portion of the polynucleotide. Mouse homologues comprising ME6 BI155218 are excluded. The polynucleotides and fragments thereof of the present invention may be of any size that is compatible with the invention, e.g., of any desired size that is effective to achieve a desired specificity when used as a probe. Polynucleotides may range in size, e.g., from the smallest specific probe (e.g., about 7-8 nucleotides) to greater than a full-length cDNA, e.g., in the case of a fusion polynucleotide or a polynucleotide that is part of a genomic sequence.

A fragment of a polynucleotide according to the invention may be used, e.g., as a hybridization probe. Probes of this type preferably have at least 7 or 8 bases, more preferably about 10, 11, 12, 13, 14 or 15 bases, and most preferably at least about 30 to about 45 bases, and exhibit about 65-100% sequence identity to part or all of the sequence coding for an inventive splice variant region. Such probes may also have 45 or more bases but again contain sequences which exhibit about 65-100% sequence identity to a sequence coding for some or all of a novel splice variant region of the invention, or a variant thereof. Because of the degeneracy of the genetic code, many sequences exist which exhibit a high degree of sequence identity to such sequences. Hybridization probes are specific to, or for, a selected polynucleotide. The phrases “specific for” or “specific to” a polynucleotide have a functional meaning that the probe can be used to identify the presence of one or more target genes in a sample. The probe is specific in the sense that it can be used to detect a polynucleotide above background noise (“non-specific binding”). In general, probes of the invention are specific for the unique splice variant sequences of the invention, and hybridize specifically to them under conditions of high stringency.

The present invention also relates to recombinant constructs that contain vectors plus polynucleotides of the present invention. Such constructs comprise a vector, such as a plasmid or viral vector, into which a polynucleotide sequence of the invention has been inserted, in a forward or reverse orientation.

Large numbers of suitable vectors are known to those of skill in the art, and many are commercially available. The following vectors are provided by way of example; Bacterial: pQE70, pQE60, pQE-9 (Qiagen), pBS, pD10, phagescript, psiX174, pBluescript SK, pBSKS, pNH8A, pNH16a, pNH18A, pNH46A (Stratagene); pTRC99a, pKK223-3, pKK233-3, pDR540, pRIT5 (Pharmacia); Eukaryotic: pWLNEO, pSV2CAT, pOG44, pXT1, pSG (Stratagene) pSVK3, pBPV, pMSG, pSVL (Pharmacia). However, any other plasmid or vector may be used as long as it is replicable and viable in the host.

In a preferred embodiment, the vector is an expression vector, into which a polynucleotide sequence of the invention is inserted so as to be operatively linked to an appropriate expression control (regulatory) sequence(s) (e.g., promoters and/or enhancers) which directs mRNA synthesis. Appropriate expression control sequences, e.g., regulatable promoter or regulatory sequences known to control expression of genes in prokaryotic or eukaryotic cells or their viruses, can be selected for expression in prokaryotes (e.g., bacteria), yeast, plants, mammalian cells or other cells. Preferred expression control sequences are derived from highly-expressed genes, e.g., from operons encoding glycolytic enzymes such as 3-phosphoglycerate kinase (PGK), □-factor, acid phosphatase, or heat shock proteins, among others. Such expression control sequences can be selected from any desired gene, e.g using CAT (chloramphenicol transferase) vectors or other vectors with selectable markers. Two appropriate vectors for such selection are pKK232-8 and pCM7.

Particular named bacterial promoters which can be used include lacI, lacZ, T3, T7, gpt, lambda PR, PL and trp. Eukaryotic promoters include CMV immediate early, HSV thymidine kinase, early and late SV40, adenovirus promoters, LTRs from retrovirus, and mouse metallothionein-I. Selection of the appropriate vector and promoter is well within the level of ordinary skill in the art.

Transcription of the DNA encoding the polypeptides of the present invention by higher eukaryotes can be increased by inserting an enhancer sequence into the expression vector. Enhancers are cis-acting elements of DNA, usually about from 10 to 300 bp that act on a promoter to increase its transcription. Representative examples include the SV40 enhancer on the late side of the replication origin bp 100 to 270, a cytomegalovirus early promoter enhancer, the polyoma enhancer on the late side of the replication origin, and adenovirus enhancers.

Generally, recombinant expression vectors also include origins of replication. An expression vector may contain a ribosome binding site for translation initiation, a transcription termination sequence, a polyadenylation site, splice donor and acceptor sites, and/or 5′ flanking or non-transcribed sequences. DNA sequences derived from the SV40 splice and polyadenylation sites may be used to provide required nontranscribed genetic elements. The vector may also include appropriate sequences for amplifying expression. In addition, expression vectors preferably contain one or more selectable marker genes to provide a phenotypic trait for selection of transformed host cells such as dihydrofolate reductase or neomycin resistance for eukaryotic cell culture, or such as tetracycline or ampi-cillin resistance in E. coli.

Large numbers of suitable expression vectors are known to those of skill in the art, and many are commercially available. Suitable vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, adeno-associated virus, TMV, fowl pox virus, and pseudorabies. However, any other vector may be used as long as it is replicable and viable in a host. Appropriate cloning and expression vectors for use with prokaryotic and eukaryotic hosts are described, e.g., by Sambrook, et al., Molecular Cloning: A Laboratory Manual, Second Edition, Cold Spring Harbor, N.Y., (1989), Wu et al, Methods in Gene Biotechnology (CRC Press, New York, N.Y., 1997), Recombinant Gene Expression Protocols, in Methods in Molecular Biology, Vol. 62, (Tuan, ed., Humana Press, Totowa, N.J., 1997), and Current Protocols in Molecular Biology, (Ausabel et al, Eds.,), John Wiley & Sons, NY (1994-1999).

Appropriate DNA sequences may be inserted into a vector by any of a variety of procedures. In general, the DNA sequence is inserted into an appropriate restriction endonuclease site(s) by procedures known in the art. Such procedures and others are deemed to be within the scope of those skilled in the art. Conventional procedures for this and other molecular biology techniques discussed herein are found in many readily available sources, e.g., Sambrook, et al., Molecular Cloning: A Laboratory Manual, Second Edition, Cold Spring Harbor, N.Y., (1989). If desired, a heterologous structural sequence is assembled in an expression vector in appropriate phase with translation initiation and termination sequences, and preferably, a leader sequence capable of directing secretion of translated protein into the periplasmic space or extracellular medium.

The present invention also relates to host cells which are transformed/transfected/transduced with constructs such as those described above, and to progeny of said cells, especially where such cells result in a stable cell line that can be used for assays of epididymis-specific receptor protein activity, e.g., in order to identify agents which modulate that activity, and/or for production (e.g., preparative production) of the polypeptides of the invention.

As representative examples of appropriate hosts, there may be mentioned: bacterial cells, such as E. coli, Streptomyces, Salmonella typhimurium; fungal cells, such as yeast; insect cells such as Drosophila S2 and Spodoptera Sf9 (and other insect expression systems); animal cells, including mammalian cells such as CHO, COS (e.g., the COS-7 lines of monkey kidney fibroblasts described by Gluzman, Cell, 23:175 (1981)), C127, 3T3, CHO, HeLa, BHK or Bowes melanoma cell lines; plant cells, etc. The selection of an appropriate host is deemed to be within the knowledge of those skilled in the art based on the teachings herein.

Introduction of a construct into a host cell can be effected by, e.g., calcium phosphate transfection, DEAE-Dextran mediated transfection, lipofection a gene gun, or electroporation (Davis, L., Dibner, M., Battey, I., Basic Methods in Molecular Biology, (1986)).

Following transformation of a suitable host strain and growth of the host strain to an appropriate cell density, the selected promoter can be induced by appropriate means (e.g., temperature shift or chemical induction) if desired, and cells cultured for an additional period. The engineered host cells are cultured in conventional nutrient media modified as appropriate for activating promoters (if desired), selecting transformants or amplifying the genes of the present invention. The culture conditions, such as temperature, pH and the like, are those previously used with the host cell selected for expression, and will be apparent to the ordinarily skilled artisan.

Cells are typically harvested by centrifugation, disrupted by physical or chemical means, and the resulting crude extract retained for further purification. Alternatively, when a heterologous polypeptide is secreted from the host cell into the culture fluid, supernatants of the culture fluid can be used as a source of the protein. Microbial cells employed in expression of proteins can be disrupted by any convenient method, including freeze-thaw cycling, sonication, mechanical disruption, or use of cell lysing agents, such methods being well known to those skilled in the art.

The polypeptide can be recovered and purified from recombinant cell cultures by conventional methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography, or the like. Protein refolding steps can be used, as necessary, in completing configuration of the mature protein. High performance liquid chromatography (HPLC) can be employed for final purification steps.

Preferably, a polypeptide of the invention is substantially purified. By “substantially purified” is meant herein that a polypeptide (or polynucleotide) is physically separated and essentially free from other polypeptides (or polynucleotides), i.e., the polypeptide (or polynucleotide) is the primary and active constituent.

In addition to the methods described above for producing polypeptides recombinantly from a prokaryotic or eukaryotic host, polypeptides of the invention can be prepared from natural sources, or can be prepared by chemical synthetic procedures (e.g., synthetic or semi-synthetic), e.g., with conventional peptide synthesizers. The same applies to polypeptides or peptide epitopes having the same immunogenicity which are coded by fragments or variants of the DNA sequences according to the invention. This is also true for oligopeptides, such as human N2, mouse N2, human A and mouse A23 (Example 1D).

Cell-free translation systems can also be employed to produce proteins of the invention, using RNAs derived from the DNA constructs of the present invention. Proteins of the invention can also be expressed in, and isolated and/or purified from, transgenic animals or plants expressing heterologous proteins of the invention. Procedures to make and use such transgenic organisms are conventional in the art.

The polypeptides and proteins according to the invention, the nucleotide sequences which code for these, including their complementary sequences, and antibodies produced on the basis of the polypeptides and proteins offer the possibility of diagnosing and, where appropriate, treating disturbances in the protein metabolism of the epididymal epithelium, and of providing new contraceptive agents.

Thus, for example, the above mentioned nucleotide sequences of the present invention can be provided with markers and used as probes for in situ hybridization in tissue diagnostics of biopsy samples or thin sections, in order to determine the physiological state of the tissue with respect to the presence and concentration of the receptor proteins according to the invention, and to compare it with standard values.

Polyclonal and monoclonal antibodies for use in the immunological detection methods can be produced in a known manner with the aid of the purified polypeptides according to the invention. Such antibodies can be produced on the basis of a complete receptor protein and on the basis of fragments and active derivatives thereof, where these have the same immunogenicity. Antibodies of the invention are specific for one or more of the peptides encoded by the novel splice regions of the invention, or for a region of a polypeptide whose secondary and/or tertiary structure is altered by a interaction with a novel splice variant peptide sequence of the invention. Any of a variety of antibodies or antigen-specific antibody fragments are included, including, e.g., chimeric, recombinant, single chain, partially or fully humanized antibodies, as well as Fab fragments, and fragments thereof.

The antibodies can be marked or labeled and used in vitro or in vivo for detection of the receptor protein according to the invention.

The receptor proteins or peptides according to the invention and biologically active derivatives, or fragments thereof having the same immunogenicity, can furthermore be used in marked or labeled or non-marked or unlabeled form as antigens for identification of autoantibodies in the sera of infertile men. This possibility is of particular importance, since it is assumed that in a large proportion of cases infertility is to be attributed to the presence of autoantibodies against essential components of the reproductive system. However, the test methods available to date measure only antibodies directed against some sperm surface antigens, and a sufficiently high titer of the antibodies must be present to allow sperm agglutination to take place. It is assumed, however, that antibodies are present in far lower titers and can cause infertility. These can be detected as an antigen with a purified or isolated novel is peptide according to the invention.

Starting from the amino acid sequences disclosed according to the invention, at least two different processes are available for isolation of antigens for the production of antibodies.

Firstly, a potentially immunogenic region of a protein sequence of interest (e.g., from a novel splice variant region), which on the one hand is relatively hydrophilic and therefore lies on the outside of the protein molecule, but on the other hand is not impaired in its steric conformation by formation of cysteine disulfide bridges or possible glycosylation sites, can be selected with the aid of a computer.

This peptide region is then synthesized, if appropriate together with flanking amino acids, subsequently coupled to carrier substances and employed as an immunogen for the induction of antibodies.

Alternatively, for example, the nucleotide sequence which codes for a peptide or polypeptide of interest can be cloned into a suitable expression plasmid vector. After subsequent transformation into suitable bacteria, these vectors allow an inducible expression of the coded peptide or polypeptide. The bacteriogenic protein or protein fragment prepared in this manner can be used directly, after purification from the bacterial extract, as an antigen for immunization for induction of antibodies.

The antibodies can be provided with a detectable marker, such as, e.g., a fluorescent molecule (fluorophor) and can be used, for example, in tissue samples to determine the presence of the epididymis-specific receptor protein in the epididymal epithelium with the aid of immunofluorescence.

The identification and characterization of a receptor protein according to the invention as a highly specific mediator molecule which is present exclusively on the cells of the epididymal epithelium of mammals and is capable of transmitting information for control of the cell function within the cells of the epithelium make it an extremely interesting candidate for diagnosis and, if appropriate, influencing of the physiology of the epididymal epithelium.

Physiological functions in which the ESRPs of the invention are involved include the following non-limiting examples:
(a) Development of an orientated forward mobility and capability for hyperactivation of the spermatozoa;
(b) Prevention of premature capacitation, i.e., readiness to perform the acrosome reaction, where decapacitation factors, which are presumably epididymal polypeptides, play a regulating role;
(c) Change in the surface antigens of the spermatozoa in order to promote binding between the spermatozoa and oocyte; and/or
(d) Change in the spermatozoal membrane in order to facilitate fusion with the ovum;
(e) Change in fluid composition and absorbtion/secreton processes within the efferent ducts and the epididymis;
(f) Change in passive spermatozoa transport along the efferent ducts and the epididymis.

The present invention provides therefore, a thereapeutic method and composition to improve the spermatozoa maturation process in the epididymis, for example by administration of an effective amount of a ligand that is lacking or is formed in an inadequate amount in the individual to be treated, in a pharmaceutically acceptable carrier or diluent.

The present invention also provides a contraceptive method and compositions which have a negative influence on the epithelium, which can lead to deterioration of spermatozoa maturation. For example, a composition of the invention may specifically disrupt post-testicular sperm maturation, and/or the epididymal luminal milieu essential for sperm storage and maintenance of the redox state. A synthetic ligand which binds firmly to a receptor protein according to the invention but induces no signal transfer or transmission is suitable, for example, for this purpose. Antibodies directed against the receptor protein (e.g., specific for a splice variant region according to the invention), or active derivatives or fragments thereof having the same immunogenicity, can furthermore be employed in order to impede binding of, and therefore signal inducement by, the ligand or ligands specific to the receptor protein by way of competitive displacement and are also a part of the present invention.

Using certain methods, such as e.g., phage display and peptide display (J. K. Scott and G. P. Smith, Searching for peptide ligand with an epitope library, Science, 249, p. 386-390 (1990)); J. J. Devlin et al., Random peptide libraries: a source of specific protein binding molecules, Science 249, p. 404-406 (1990)), Evolutive Biotechnology (M. Eigen, Self organization of matter and the evolution of biological macromolecules, Die Naturwissenschaften, 58, p. 465-523 (1971); M. Eigen, Automated molecular evolution, Max-Planck Institute of Biophysical Chemistry (1991)), one is able to identify synthetic ligands which have the ability to bind specifically and with a high affinity to such a receptor protein (e.g., to a splice variant region) and to act either agonistically or antagonistically on the signal transfer capability thereof (see, e.g., Example 6). Molecules by means of which the physiology of cells which express the receptor protein can be influenced positively (therapeutically) or negatively (contraceptively) can be provided by this route.

Alternatively, an ESRP of the invention, expressed in a host cell, as described herein, may be expressed to retain the transmembrane and, optionally, the cytoplasmic region of the native variant, to be anchored in the membrane of the host cell, and the cells carrying the ESRP may be used as such in the screening or diagnostic assay. Alternatively, the receptor may be a component of membrane preparations, e.g., in solubilized and/or reconstituted form.

A protein or peptide of the invention (e.g., from a splice variant region), derivative or analogue thereof, may be immobilized on a solid support and may, as such, be used as a reagent in the screening methods of the invention. The ESRP, derivative or analogue may be used in membrane-bound form, i.e., bound to whole cells or as a component of membrane preparations immobilized on a solid support.

The solid support employed in the screening methods of the invention preferably comprises a polymer. The support may in itself be composed of the polymer or may be composed of a matrix coated with the polymer. The matrix may be of any suitable material such as glass, paper or plastic. The polymer may be selected from the group consisting of a plastic (e.g., latex, a polystyrene, polyvinylchloride, polyurethane, polyacrylamide, polyvinylalcohol, nylon, polyvinylacetate, and any suitable copolymer thereof), cellulose (e.g., various types of paper, such as nitrocellulose paper and the like), a silicon polymer (e.g. siloxane), a polysaccharide (e.g. agarose or dectran), an ion exchange resin (e.g. conventional anion or cation exchange resins), a polypeptide such as polylysine, or a ceramic material such as glass (e.g. controlled pore glass).

The physical shape of the solid support is not critical, although some shapes may be more convenient than others for the present purpose. Thus, the solid support may be in the shape of a plate, e.g. a thin layer or microtiter plate, or a film, strip, membrane (e.g. a nylon membrane or a cellulose filter) or solid particles (e.g. latex beads or dextran or agarose beads).

Ligands of the invention can take a variety of forms, e.g., peptides, antibodies or antigen-binding fragments, small molecules (of any of a variety of conventionally recognized types), or the like.

It is furthermore contemplated to locate the ligand-binding site on an ESRP of the invention, for instance by preparing deletion or substitution derivatives of the native ESRP (as described herein) and incubating these with ligands known to bind the full-length ESRP and detecting any binding of the ligand to the ESRP deletion derivative. Once the ligand-binding site has been located, this may be used to acquire further information about the three-dimensional structure of the ligand-binding site. Such three-dimensional structures may, for instance, be established by means of protein engineering, computer modeling, NMR technology and/or crystallographic techniques. Based on the three-dimensional structure of the ligand-binding site, or can design substances which are agonists of antagonist to the ESRP molecule.

Potential antagonists or inhibitors of the invention also include isolated antisense oligonucleotides, or antisense constructs which express antisense oligonucleotides, both of which classes of molecules can be prepared using conventional technology. Antisense technology can be used to control gene expression through methods based on binding of a polynucleotide to DNA or RNA. Without wishing to be bound to any particular mechanism, types of antisense oligonucleotides and proposed mechanisms by which they function include, e.g., the following: An antisense oligonucleotide (e.g., an RNA, DNA, PNA etc. oligonucleotide) of from about 10 to 40 base pairs in length can be designed on the basis of a unique sequence from the splice region of the invention. The antisense oligonucleotide can hybridize to the mRNA and block translation of the mRNA molecule into an ESRP polypeptide (see e.g., Okano, J. Neurochem., 56:560 (1991); Oligodeoxynucleotides as Antisense Inhibitors of Gene Expression, CRC Press, Boca Raton, Fla. (1988)). Alternatively, an oligonucleotide can be designed to be complementary to a region of the gene involved in transcription (see, e.g, Lee et al., Nucl. Acids Res., 6:3073 (1979); Cooney et al, Science, 241:456 (1988); and Dervan et al., Science, 251: 1360 (1991)), thereby preventing transcription of a specific ESRP. For further guidance on administering and designing antisense, see, e.g., U.S. Pat. Nos. 6,200,960, 6,200,807, 6,197,584, 6,190,869, 6,190,661, 6,187,587, 6,168,950, 6,153,595, 6,150,162, 6,133,246, 6,117,847, 6,096,722, 6,087,343, 6,040,296, 6,005,095, 5,998,383, 5,994,230, 5,891,725, 5,885,970, and 5,840,708.

Antisense polynucleotides can comprise modified, nonnaturally-occurring nucleotides and linkages between the nucleotides (e.g., modification of the phosphate-sugar backbone; methyl phosphonate, phosphorothioate, or phosphorodithioate linkages; and 2′-O-methyl ribose sugar units), e.g., to enhance in vivo or in vitro stability, to confer nuclease resistance, to modulate uptake, to modulate cellular distribution and compartmentalization, etc. Any effective nucleotide or modification can be used, including those already mentioned, as known in the art, etc., e.g., disclosed in U.S. Pat. Nos. 6,133,438; 6,127,533; 6,124,445; 6,121,437; 5,218,103 (e.g., nucleoside thiophosphoramidites); U.S. Pat. No. 4,973,679; Sproat et al., “2′-O-Methyloligoribonucleotides: synthesis and applications,” Oligonucleotides and Analogs, A Practical Approach, Eckstein (ed.), IRL Press, Oxford, 1991, 49-86; Iribarren et al., “2′O-Alkyl Oligoribonucleotides as Antisense Probes,” Proc. Natl. Acad. Sci. USA, 1990, 87, 7747-7751; Cotton et al., “2′-O-methyl, 2′-O-ethyl oligoribonucleotides and phosphorothioate oligodeoxyribonucleotides as inhibitors of the in vitro U7 snRNP-dependent mRNA processing event,” Nucl. Acids Res., 1991, 19, 2629-2635. Effective amounts of antisense oligonucleotides as described above can be administered to a patient in need thereof by conventional means.

Antisense oligonucleotides can also be delivered to cells via, e.g., plasmids or other vectors, wherein the antisense sequence is operably linked to an expression control sequence. In this manner, RNA or DNA antisense is expressed in a cell and inhibits production of ESRPs of the invention. A total length of about 36 nucleotides can be used in cell culture with cationic lipisomes to facilitate cellular uptake, but for in vivo use, preferably shorter oligonucleotides are administered, e.g., about 25 nucleotides.

In another embodiment, ribozymes corresponding to specific sequences, e.g., polynucleotides comprising the inventive splice regions or fragments thereof, can be introduced into cells such that they cleave specific ESRP coding sequences. Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. Ribozyme molecules designed to catalytically cleave target gene mRNA transcripts can also be used to prevent translation of target gene mRNA and expression of target gene. (See, e.g., PCT International Publication WO90/11364, published Oct. 4, 1990; Sarver et al., 1990, Science 247:1222-1225). While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. For example, there are hundreds of potential hammerhead ribozyme cleavage sites within the nucleotide sequence of PDE4D7 sequences of the invention. Preferably the ribozyme is engineered so that the cleavage recognition site is located near the 5′ end of the target mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in target gene.

As in the antisense approach, the ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes, unlike antisense molecules, are catalytic, a lower intracellular concentration is required for efficiency.

Any of the agents discussed hereinabove (e.g., antagonists or inhibitors of a receptor polypeptide of the invention, ligands, including antibodies or antigen-binding fragments, antisense oligonucleotides, etc.) can be formulated as a pharmaceutical composition, comprising one or more conventional pharmaceutically acceptable carriers. Such pharmaceutical carriers can be used, e.g., in the diagnosis of a male reproductive disorder, for the treatment of a male reproductive disorder, for male contraception, or the like.

In accordance with the present invention, an antibody or antigen-binding fragment can be present in a kit, where the kit includes, e.g., one or more antibodies or antigen-binding fragments, a desired buffer, detection compositions, proteins to be used as controls, etc. The invention also relates to a kit which includes one or more nucleic acids or oligonucleotide fragments according to the invention, as well as, e.g., a desired buffer, detection compositions, nucleic acids to be used as controls, etc.

### BRIEF DESCRIPTION OF THE DRAWINGS

FIG. 1 shows an alignment of deduced amino acid sequences of the longest HE-6-type polypeptides predicted from human, mouse and rat cDNAs. The ectodomain is presented on the left panel, ending with the predicted proteolysis site; the endodomain is presented on the right. Sequences were aligned for maximal sequence homology. Residues which are identical to the human sequence are shown on a grey background; species differences are highlightened by white and black background. The additional three amino acids (GTP) encoded by a 9 nt mini-exon are inserted at position 6567 in the human sequence. The dotted line marks the 51 amino acid C-terminal truncation observed in the □E24 splice variant. N2 and A/A23 oligopeptide sequences employed for antibody production are boxed. The length of the different domains are indicated above the sequences as black bars (SP=signal peptides; splice region=region of high variability resulting from alternative splicing; STP domain=mucin-like serine/threonine/proline-rich sequence; GPS=G-protein-coupled receptor proteolysis site; TM1-TM7=seven-transmembrane domains).

FIG. 2 illustrates the genomic organization of the 5′ region of the HE6 gene on human X-chromosome (Genbank accession no. AC021139) and alignment of splice variants cloned from human, mouse (ME6), and rat (RE6) epididymal RNA. Differential mRNA splicing is suggested in a 185 nt-spanning sequence directly downstream of exon E3, containing the start codon (ATG, nt 164-166) and encoding the signal peptide. The alignment covers human exons E3-E8; the predicted amino acid sequences are always in frame; note that exon E7 is present in all splice variants identified. The upper panel (grey shaded boxes) shows the exon organization of seven new human HE6 variants obtained by 5′RACE and RT-PCR and of former sequence X81892 (Osterhoff et al. 1997 DNA Cell Biol 16, 379-389). Numbering refers to the longest HE6 variant which was 100 nt longer and comprised the 9 nt micro-exon E5b (acc.no.xxx). Five splice variants of ME6 (middle panel, hatched boxes; B1155218 from EST databases) and three variants of RE6 (lower panel, dotted boxes) all lacked exon E6. Different from the human variants, one ME6 and one RE6 variant also lacked exon E4.

FIG. 3 shows a comparison of the male reproductive tract of HE6 knock out animals (A) with wilde-type (B) animals. The tissues slices are stained with Haematoxilin-Eosin (method according to Romeis 1989: Mikroskopische Technik; Urban und Schwarzenberg, 17th edition). The accumulation of spermatozoa within the distal efferent ducts is well visible. No sperm is found in the parts of the epididymis. (magnification 25×; slides composed of several detail screens).

In the foregoing and in the following examples, all temperatures are set forth uncorrected in degrees Celsius; and, unless otherwise indicated, all parts and percentages are by weight.

### EXAMPLES

### Example 1

### Methods

A. Tissues

Human epididymides were obtained from local hospitals where orchiectomy was carried out for treatment of prostate carcinoma. Rodent tissues were collected from freshly killed laboratory animals (NMRI male mice, aged 5 to 60 days; adult Wistar male rats, body weight approximately 400 g). Tissues were shock frozen in liquid nitrogen and stored at −80° C., or immersion-fixed in Bouin's solution and stored in 70% ethanol.

B. Enzymes and Specific Biochemicals

O-Glycanase from S. pneumoniae was purchased from Glyko, Nr Bicester, UK, (recombinant from E. coli; catalogue no. 80090); N-glycosidase F from F. meningosepticum (recombinant from E. coli) and Neuraminidase from A. ureafaciens were purchased from Roche Molecular Biochemicals, Mannheim, Germany. Protein concentrations were determined employing the Dc-Kit (Bio-Rad, München, Germany). Biotinylated anti-rabbit IgG (Dianova, Hamburg, Germany), peroxidase-antiperoxidase (PAP) complexes from rabbits (Dianova), avidin-biotin (ABC) complexes (Vector Laboratories, Burlingame, Calif., USA), and 3,3′Diaminobenzidine (Dako, Hamburg, Germany) were employed in immunohistochemistry anti-rabbit horse-radish peroxidase-conjugated antibody (Sigma Deisenhofen, Germany) combined with the CL-HRP substrate system (Pierce Chemical Company; Rockford, Ill.) were employed in immunoblotting.

C. RNA Extraction and Northern Blot Analysis

Total RNA was extracted from frozen mouse tissues by the RNAclean method (AGS, Heidelberg, Germany), and poly(A)+RNA was isolated using standard procedures employing oligo(dT) cellulose type 7 (Amersham Pharmacia Biotech, Freiburg, Germany). For Northern blot analysis following standard protocols, 3 μg of poly(A)+RNA per lane were separated on denaturing agarose gels and transferred to Hybond N membrane (Amersham). A cDNA-fragment containing 817 base pairs (nt 797-1613) of the mouse HE6-homologue Me6 (accession no. xxx) and a 917 bp actin cDNA fragment (nt 72-988, accession no. X03765) were 32P-labelled according to Feinberg and Vogelstein, 1983 employing the Prime-It II random primer labelling Kit (Stratagene, Amsterdam, Netherlands), denatured and employed as hybridization probes as described (Kirchhoff et al., 1990). Autoradiograms were exposed to Kodak BioMax MS autoradiography film (Amersham) and developed after two hours to five days of exposure, depending on the probe.

D. cDNA Synthesis and PCR Amplification

For standard RT-PCR, oligo(dT)-primed cDNA was synthesized in a 20 μl reaction from 5 μg total epididymal RNA using 200U of Superscript II reverse transcriptase (Life Technologies, Eggenstein, Germany), 1 mM dNTP, and 0.5 μg oligo(dT)12-18. Incubation was for 60 min at 45° C. For “inverse” PCR, cDNA synthesis with gene specific primers was performed as described by Gebhardt et al., 1999 J. Reprod Fertil 116, 391-402). 3′-RACE was performed essentially as described by Chenchik et al. 1996 Biotechniques 21, 526-534). PCR amplification was performed in a 50 μl volume with 0.5U Biotherm Taq-Polymerase (Genecraft, Munster, Germany), 1 μl single-stranded cDNA, 200 nM dNTP, 20 pMol each of primers in the PCR buffer provided (Genecraft). Sequences of all primers and PCR conditions are listed in Table 1. PCR products were isolated and ligated into the TA-cloning vectors pCRII (Invitrogen, Heidelberg, Germany) or pGEM-T Easy (Promega). Plasmid DNA was sequenced from both strands (MWG, Ebersberg, Germany). Sequence alignments were performed using the DNASTAR software.

E. Oligopeptides and Primary Antibodies

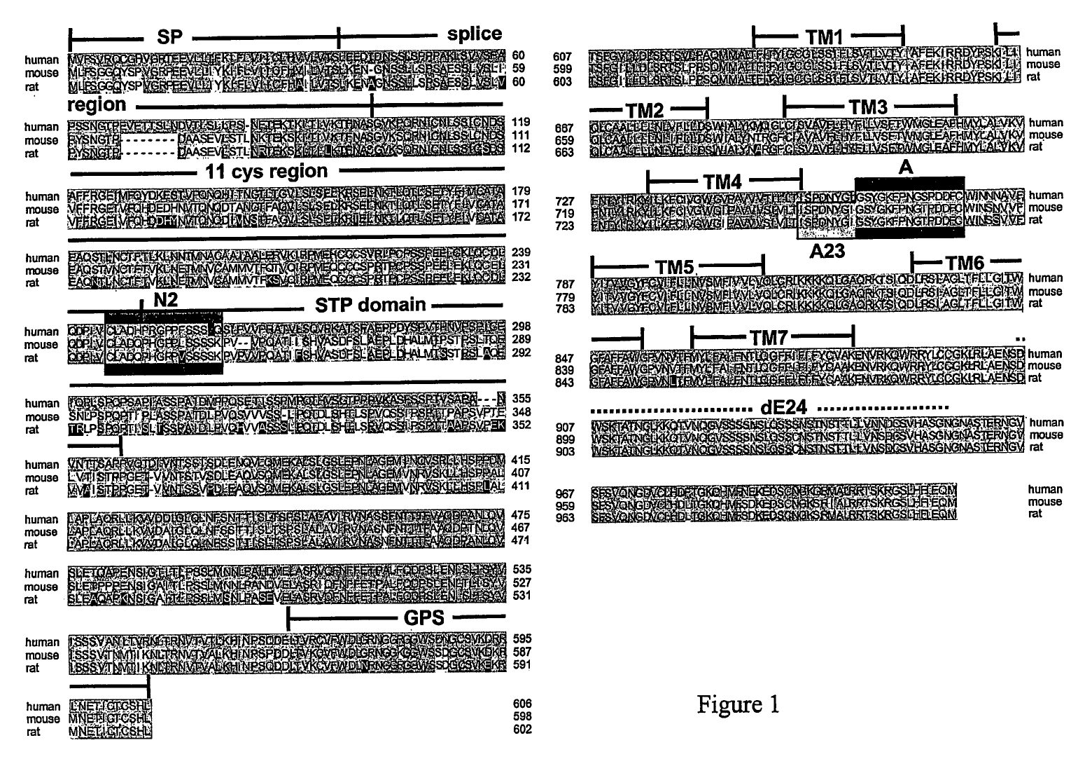

Chemosynthetic oligopeptides were obtained (FZB and Pineda-Antikörper Service, Berlin, Germany) according to the amino acid sequences deduced from human and mouse cDNAs (human HE6 GenBank accession no. X81892). Oligopeptide sequences were species-adjusted to improve epitope recognition of antibodies. N-terminal peptides were as follows. Human N2: 15-mer (242-256) NH2-CLADHPRGPPFSSSQ-COOH and mouse N2: 16-mer (237-252) NH2-CLADQPHGPPL SSSSK-COOH. (FIG. 1). C-terminal peptides were as follows. Human A: 15-mer (761-775) NH2-GSYGKFPNGSPDDFC-COOH; mouse A 23: 23-mer (748-770) NH2-ISPDNYGIGSYGKFPNGTPDDFC-COOH. Peptides were conjugated to keyhole limpet hemocyanin as a carrier using terminal cysteine residues for coupling, and the conjugates employed to immunize female rabbits. Immune sera were obtained after 120 days. Monospecific purification of polyclonal antibodies was performed by affinity chromatography (Pineda-Antikörper-Service).

F. Epididymal Membrane Preparation

Epididymal membrane suspensions were prepared from two individual patient tissues and from pooled rodent epididymides collected from at least four animals per experiment. Membranes were prepared essentially as described by Muller et al., 2000 Brain Res Mol Brain Res 75, 321-329. Briefly, frozen epididymides were pulverized under liquid nitrogen in a dismembrator (Braun, Melsungen, Germany) and suspended in 10× vol. of ice-cold homogenization buffer containing 50 mM Tris-Cl, pH 7.5, 1 mM EDTA, 1 mM dithiothreitol, and “complete” protease inhibitor as suggested by the supplier (Roche). Suspensions were homogenized by ten strokes in a Potter-Elvehjem homogenizer and centrifuged to separate debris (3000×g, 5 min, 4° C.). Supernatant fractions were centrifuged at 100,000×g, 30 min, 4° C. Pellets were resuspended in 5 mM Tris, pH 7.5 containing “complete”, and membrane suspensions stored as aliquots at −80° C. The corresponding supernatants were stored as “cytosolic” fractions.

G. Deglycosylation of Epididymal Membrane Proteins

Membrane proteins were enzymatically deglycosylated as described by Goldberg et al., 2000 J. Biol Chem 275, 24, 622-24629) with the following modifications. 20-40 μg of epididymal membrane proteins were denatured in 0.3% SDS, 0.3% β-mercaptoethanol, 1% NP-40, and complete (Roche) at 95° C. for 5 min. Samples were digested o/n in a 20 μl reaction employing 10 mU neuraminidase, 1.5 mU O-glycanase, and 100 mU N-glycosidase F in 50 mM sodium phosphate, pH 5.0. Enzymes were inactivated at 60° C. for 5 min prior to SDS-PAGE separation of proteins.

H. Western Blot Analysis

For the analysis of the ectosubunits, membrane proteins were denatured in Laemmli sample buffer and separated on 8% or 10% Laemmli polyacrylamide gels. For the analysis of the endosubunits, membrane preparations were solubilized for 1 h at ambient temperature in 7M urea, 2M thiourea, 4% Chaps, 1% Triton X-100, 1% DTT, 20 mM Tris, pH 9.5, and complete (Roche). After addition of β-mercaptoethanol (2% end concentration) samples were separated on 12% urea (7M) gels. Proteins were transferred to PVDF membranes (Millipore, Eschborn, Germany) in a continuous buffer system using a semi-dry blotter (Phase, Lubeck, Germany). Immunodetection of proteins was carried out by standard procedures, employing affinity purified anti-human N2- and A-antibodies at a dilution of 1:300, and anti-mouse N2- and A23-antisera from rabbits at dilutions of 1:35000 and 1:5000, respectively. Immunopositive bands were detected employing anti-rabbit horse-radish peroxidase-conjugated antibody (1:1000) combined with the CL-HRP substrate system at a dilution of 1:5 and exposure to Kodak-X-Omat (XAR) autoradiography film (Amersham). Specificity of antibody binding was confirmed by comparison with preimmune sera and by competition with the oligopeptides as described by Derr et al., 2001 Reproduction 121, 435-446.

I. Immunohistochemistry

Bouin-fixed human and mouse epididymides were embedded in paraffin wax and 5 μm sections prepared for immuno-localization of HE6. Paraffin sections were dewaxed, and finally submersed in phosphate-buffered saline. As first antibodies, species-adjusted N2 and A-antibodies were employed at dilutions of 1:500 in the case of affinity purified antibodies and of 1:2000 in the case of antisera; the corresponding pre-immune sera served as negative controls. A conventional double-PAP-ABC procedure (Davidoff and Schulze, 1990) was adopted with the modifications as described (Balvers et al., 1998). Briefly, as second (bridge) antibodies biotinylated anti-rabbit IgG were used at a dilution of 1:500. In the next steps peroxidase-antiperoxidase complexes from rabbits (1:500), avidin-biotin complexes, and diaminobenzidin reagent were used for detection. Tissue sections were investigated using normal brightfield microscopy (Nikon, Japan) and images captured with a Leica DC 200 digital camera (Leitz, Bensheim, Germany).

### Example 2

### Cloning of Rodent HE6-Homologous cDNAs

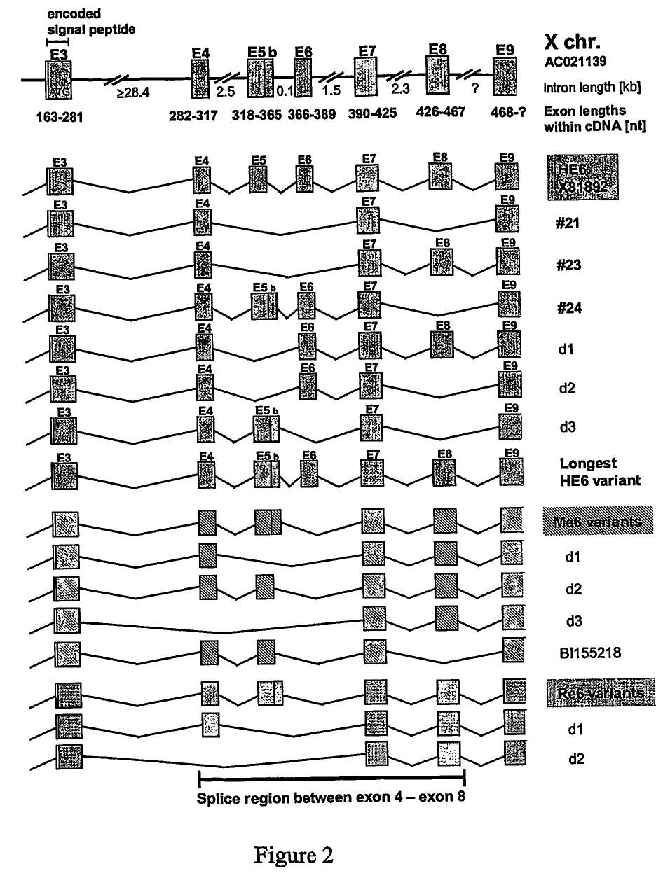

The open reading frame of the HE6 predicted a novel member of the LNB-TM7 (long N-terminal extensions, family B, seven-transmembrane domain) subfamily of GPCRs (G protein-coupled receptors) with no known close similarity to any sequences present in the databases. The highly conserved TM7 domains had been identified earlier (Osterhoff et al., 1997 DNA Cell Biol 16, 379-389). A combined strategy, using an “inverse” RT-PCR technique as well as homology PCR screening, and database screening (see Table 1, EST clones) was employed here to clone the complete rodent cDNAs, including the ectodomain-encoding sequences. Rat and mouse HE6-homologous cDNA sequences were compiled, chimerization of the cDNAs ruled out by amplification of overlapping segments, and the sequences deposited in the EMBL database (accession no. xxx and xxx). The alignment of the deduced amino acid sequences is shown in FIG. 1. A similar cloning strategy was applied to human epididymal cDNA to possibly extend the known 5′-end of the HE6 mRNA. As a result, the published sequence (Osterhoff et al., ibid) was extended by 100 nucleotides (accession no. xxx), including nine additional nucleotides from an alternative splice donor (FIG. 2). The extended sequence contained an open reading frame upstream from the first in-frame ATG triplett.

The amino acid sequences as predicted by the longest cDNAs of each species is shown in FIG. 1. Compared to the highly conserved TM7 and intracellular C-terminal domains (95% overall sequence identity between human and rodents), the conservation within the predicted ectodomains was much lower (<70% overall sequence identity). Nevertheless, upstream of the GPS (GPCR proteolysis site) motif, the following regions could be identified in each species: i) a 37-amino acid signal peptide-coding region (67-70% identity), ii) a region of highly variable length, probably resulting from alternative mRNA splicing between exons 4 and 8 (<50%, nucleotides 282467 of the longest HE6 cDNA sequence; compare FIG. 2), iii) an invariable region encoding 11 cysteines (77% sequence identity), and iv) an STP (serine/threonin/proline)-rich domain predicting 19-25 O-glycosylation sites (which may form a mucin-like stalk). The lowest degree of interspecies conservation (<45%) was observed within this mucin-like domain which, however, showed no extensive length differences. The GPS motif again displayed a high degree of conservation (approximately 85% identity between human and rodents).

The cDNA sequences immediately following the signal peptide-coding sequences were characterized by a considerable length heterogeneity in each species (FIG. 2). We identified eight variants in the human, four in the mouse (plus one in an EST clone), and three in the rat. When aligned with the Homo sapiens genomic contig database (clone AC021139), the 5′-region of the human HE6 cDNA was divided into multiple short exons separated by introns of varying lengths, suggesting that the variants originated from alternative splicing of mini-exons (FIG. 2). All predicted human splice sites were conform to the GT-AG rule, with the exception of the splice donor in exon 5b. Colinear variants were observed in mouse and rat epididymal mRNAs and the open reading frames were preserved in each species (FIGS. 1, 2).

Employing a 3′-RACE procedure to human and mouse cDNAs, an additional splice variant was detected affecting the intracellular C-terminal domains. In the alternative transcripts detected the predicted C-terminal domains were shortened by 51 triplets (FIG. 1). Multiple tissue Northern blot analysis employing mouse poly(A)+RNA extracts (as described in Example 1) revealed a highly restricted expression pattern of the HE6-homologous mRNA. A prominent band of approximately 5 kb was exclusively observed in the proximal epididymis of the mouse, congruent with the patterns previously obtained by Northern blot analysis in the human and the rat (Osterhoff et al., ibid). However, faint signals of approximately 5 kb were also observed in poly(A)+RNA extracts of mouse spleen and brain after prolonged exposure of the blot. Other tissues tested included distal epididymis, heart, liver, kidney, lung, whole embryo and adult testis.

TABLE 1

Oligonucleotide primer sequences

| | | | |
|---|---|---|---|
| gene | PCR | gsp | PCR program |

| | | | |
|---|---|---|---|
| HE6 | PCR-1 | hDa: TCCAGGAGGAAGACCAG (SEQ ID NO:33) | annealing 50° C. |
| | | hA6s: CCTGGAAGAAGATACTG (SEQ ID NO:34) | 40 cycles |
| | 5′RACE-2 | cDNA: hBa: TGGCATTCACTCTGATC (SEQ ID NO.35) | annealing 55° C. |
| | | inverse PCR h5′RACEs: CTTCTTTGGCTCTGGCTG | 34 cycles |
| | | (SEQ ID NO.36) | |
| | | h5′RACEa: ATAGTCGTGTTTGAAAAGTTC | |
| | | (SEQ ID NO:37) | |
| | PCR-2 | hA6s: CCTGGAAGAAGATACTG (SEQ ID NO:38) | annealing 55° C. |
| | | hA3a: AGCAAGACAGACAATGG (SEQ ID NO:39) | 34 cycles |
| Re6 | 5′RACE-1 | cDNA: rK2a: TGATCGGCAAGGCAGACAATG | TD 61-51° C. |
| | | (SEQ ID NO:40) | 40 cycles |
| | | inverse PCR: mR18s: AGTGCTGCTGTTCCCCGAGG | |
| | | (SEQ ID NO:41) | |
| | | mR18a: AGTTTACCGTGCTTTGGGCCTCT | |
| | | (SEQ ID NO:42) | |
| | 5′RACE-2 | cDNA: | TD 61-51° C. |
| | | mK17a: AGACAATGGGATCCTGCAGTTCACAC | 40 cycles |
| | | (SEQ ID NO:43) | |
| | | inverse PCR: mR18s: AGTGCTGCTGTTCCCCGAGG | |
| | | (SEQ ID NO:44) | |
| | | mR18a: AGTTTACCGTGCTTTGGGCCTCT | |
| | | (SEQ ID NO:45) | |
| | PCR-1 | mK23s: ATGCTTTTCTCTGGTGGGCAGTAC | TD 65-55° C. |
| | | (SEQ ID NO:46) | 30 cycles |
| | | rK2a: TGATCGGCAAGGCAGACAATG (SEQ ID NO:47) | |
| | PCR-2 | mK9s: CATGGCCCACCGTTATCGTCT (SEQ ID NO:48) | TD 55-45° C. |
| | | hF1a: TGAAGGCACACATCTCC (SEQ ID NO:49) | 30 cycles |
| | PCR-3 | mME62s: GAATAGTGATTGCTCAGTGC | TD 55-45° C. |
| | | (SEQ ID NO:50) | 30 cycles |
| | | mME62a: CTGGTAAGTTATCACATTAG | |
| | | (SEQ ID NO:51) | |
| | PCR-4 | mK24s: GCCGGATAGCGCTCAGAAG | TD 61-51° C. |
| | | (SEQ ID NO:52) | 30 cycles |
| | | rK1a: ATGCTCTAGGACTCGGAACAATCAG | |
| | | (SEQ ID NO:53) | |
| | PCR-5 | mK25s: TGGCCAGACTTCATCCCTAAT | TD 61-51° C. |
| | | (SEQ ID NO:54) | 30 cycles |
| | | r3′ENDEa: GTGCAAATTTATTTTATTGATTTTATCA | |
| | | (SEQ ID NO:55) | |

ESTs: BF523552, BF388060, AI502386

| | | | |
|---|---|---|---|
| Me6 | 5′RACE-1 | cDNA: mK17a: | TD 61-51° C. |
| | | AGACAATGGGATCCTGCAGTTCACAC | 40 cycles |
| | | (SEQ ID NO:56) | |
| | | inverse PCR: | |
| | | mR18s: AGTGCTGCTGTTCCCCGAGG | |
| | | (SEQ ID NO:57) | |
| | | mR18a: AGTTTACCGTGCTTTGGGCCTCT | |
| | | (SEQ ID NO:58) | |
| | 5′RACE-2 | cDNA: mR0a: GGAGGTGTGCTGATGGTGAC | TD 65-55° C. |
| | | (SEQ ID NO:59) | 40 cycles |
| | | inverse PCR: | |
| | | mR3s: TCTCCTCAGCCTACGATCCCCCT | |
| | | (SEQ ID NO:60) | |
| | | mR13a: AAGACGATAACGGTGGGCCATGC | |
| | | (SEQ ID NO:62) | |
| | PCR-1 | mK20s: AGTTTCCTCCCTATTTCCTCTGA | TD 61-51° C. |
| | | (SEQ ID NO:63) | 30 cycles |
| | | mR1a: GATAACGGTGGGCCATGC (SEQ ID NO:64) | |
| | PCR-2 | mK1s: GCATGGCCCACCGTTATC (SEQ ID NO:65) | TD 55-45° C. |
| | | hF1a: TGAAGGCACACATCTCC (SEQ ID NO:66) | 30 cycles |
| | PCR-3 | mK6s: ACTCCACTAACTCCACCACACTCC | TD 55-45° C. |
| | | (SEQ ID NO:67) | 30 cycles |
| | | m3′ENDEa: TTTTAACTGCAAATTGATTTTT | |
| | | (SEQ ID NO:68) | |

ESTs: AW211574, AW212196, AV242919
gsp: gene specific primer, h, m, r: primers derived from human, mouse, rat cDNA sequences, s, a: sense, antisense, TD: touch down

### Example 3

### Western Blot Analysis of Human and Rodent HE6 Ectosubunits

Membrane proteins and “cytosolic” fractions were prepared from human and rodent epididymides to identify the HE6 receptor proteins. By analogy to the CIRL (calcium-independent receptor of alpha-latrotoxin) receptor, HE6 was assumed to consist of two subunits which would separate as two protein bands under standard denaturing SDS-PAGE conditions. A 15-mer epitope, located N-terminally of the STP region (N-2-epitope, FIG. 1), was chosen to raise antibodies against the predicted ectosubunits of human and rodent epididymides. To optimize antibody binding, the oligopeptides had been “species-adjusted”, based on the predicted human and mouse amino acid sequences, respectively (see Example 1 and FIG. 1). A prominent immunopositive band of approximately 180 kDa was observed in epididymal membrane protein preparations of all three species. Heat denaturation of the protein samples had no detectable effect on the migration pattern of this band. A band of the same apparent mass was observed in the corresponding cytosolic fractions, albeit at much lower concentrations. Specificity of antibody binding was shown by comparison with the corresponding preimmune serum and by its competition employing the N2 oligopeptides.

The apparent molecular mass of 180 kDa by far exceeded the predicted mass of the entire HE6 receptors, and even more so that of the ectosubunits alone. The diffuse appearance of the bands was indicative of a degree of micro-heterogeneity caused by protein glycosylation. Indeed, the 180 kDa immunopositive bands of each of the three species were highly sensitive to enzymatic protein deglycosylation, the removal of Asn-linked carbohydrates by PNGase F-digestion resulting in a significant increase of electrophoretic mobility. Also, these smaller bands were stained much more intensely the N2 antibodies, suggesting that the deglycosylation procedure had unmasked the corresponding epitopes.

A detailed analysis employing combinations of three different glycohydrolases followed by Western blot analysis was performed in the human. Neuraminidase treatment alone resulted in a marked reduction of the apparent molecular mass, predicting a high degree of peripheral sialylation. A combination of neuraminidase and O-glycanase suitable to cleave O-linked carbohydrates, resulted in a shift to an approximately 150 kDa band. PNGase F-digestion alone revealed an immunopositive band of approximately 100 kDa. A combination of all three glycohydrolases reduced the apparent mass of the ectosubunit by more than 100 kDa, resulting in an immunopositive band of approximately 70 kDa. This apparent mass was in good agreement with the caluclated mass of the ectosubunit assuming that the HE6 peptide backbone was cleaved within the GPS motif.

### Example 4

### Analysis of the Human and Rodent HE6-endosubunits

Antibodies were raised against several epitopes of the predicted endosubunits, including the so-called A- and A23-epitopes located within the second extracellular loop of the TM7 domains (FIG. 1). None of these antibodies reacted with the 180 kDa ectosubunits in any of the three species. Instead, under standard SDS-PAGE conditions, including a 95° C. denaturation step, they reacted with very large aggregates >250 kDa, not entering the separation gels. Extensive protein solubilization at ambient temperature and separation in 7M urea gels, however, resulted in an approximately 40 kDa band in the human and in an approximately 25 kDa band in rodents. These bands were absent from parallel blots employing the corresponding preimmune sera, and were competed for by the A oligopeptides. Although from their predicted amino acid sequences the human and rodent HE6 endosubunits were expected to be nearly identical (compare FIG. 1), the immunopositive bands of rodent membrane protein preparations migrated significantly faster than the human, suggesting that they represented translations products of two different HE6 splice variants (compare FIG. 1).

The intracellular C-terminus of these proteins contains all the structural prerequisites for G-protein coupling, consistent with the role of these proteins in a signal transduction pathway controlling epididymal function and male fertility.

### Example 5

### Immunohistochemical Localization of HE6 in Human and Mouse Epididymis

Sagittal cross-sections of human and mouse epididymis were employed to localize the HE6 receptor. The majority of ductular cross-sections in the human caput region which represented ductuli efferentes were intensely stained, although immunonegative tubuli were occasionally observed directly adjacent to the positive tubuli, and a mosaic-type staining pattern was sometimes seen even within the same ductular cross-section. Cross-sections of the human distal caput and corpus were immunopositive as well, however, the staining intensity was gradually decreased. In the mouse epididymis, maximum immunostaining was observed in the initial segment of the caput region where it appeared to be restricted to the long stereocilia protruding into the lumen. The epithelia of the efferent ducts appeared immuno-positive as well. However, all epididymal segments located distally of the mouse initial segment appeared unstained.

In both species, HE6-related immunoreactivity was associated with the apical, adluminal pole of efferent ducts and proximal epididymal duct. In the human proximal caput region, at a higher magnification, the HE6-related immunoreactivity was observed only at the apical border of the epithelial cells. The cilia protruding into the lumen of the human efferent ductules (“kinocilia) appeared to be free of any immunopositive material. In the ductular cross-sections of the epididymal duct proper, on the other hand, the long “stereocilia” were strongly immunopositive. Anti-N2- and anti-A-antibodies revealed identical staining patterns in both species, indicative of a spatial coincidence of both epitopes.

### Example 6

### Use of a Receptor Protein of the Invention or of a cDNA Encoding for a Receptor Protein of the Invention for Isolating Specific Ligands

To isolate specific ligands for a receptor protein according to the invention, the N-terminal extracellular domain of the amino acid sequence of HE6 according to SEQ ID NO: 30 of a receptor protein is prepared in a eukaryotic expression system such as the cell lines COS-7, HEK 393 and MDCK. For this purpose, the cDNA region which codes for this domain (corresponding to position 91-3211 of the nucleotide sequence according to SEQ ID NO: 22 is provided on the 3′ end with a flag sequence, i.e., an oligonucleotide sequence, which codes for a known, highly specific peptide epitope, and cloned into, e.g., the expression vector pcDNA3.1, pRc/CMV or pTracer-CMV (Invitrogen, San Diego, Calif., USA). After transfection of the cell lines with the expression vector described, the fusion product is obtained by known processes and purified by affinity chromatography using immobilized antibodies directed against the flag eptitope.

The fusion product is then employed as a probe in a conventional protein screening process (cf. J. Sambrook, E. F. Fritsch and T. Maniatis, Molecular Cloning: A Laboratory Manual, Cold Spring Harbor Laboratory, Cold Spring Harbor, N.Y., chapter 2 (1989)) using a cDNA expression library from the human testis prepared in a lambda baceteriophage. For this, about 1 million independent cDNA bacterial clones which express testicular products and therefore potential ligands by IPTG induction are transferred to nylon membrane filters and incubated with the recombinant receptor-binding domain under suitable conditions (see above). The receptor fragments which are not bound specifically are then removed under stringent washing conditions, so that only specific binding complexes which can be rendered visual via the flag epitope with conventional antibody detection systems (system using alkaline phosphatase, Sigma, Deisenhofen) are present on the filters. The phage colonies identified in this manner are isolated and purified and subjected to a sequence determination.

In order to ascertain whether the ligands discovered can induce a signal transduction by the receptor according to the invention and are therefore suitable for simulating maturation of spermatozoa in subfertile mammals, cultures of the cell lines mentioned above transfected with the total cDNA construct with and without a flag epitope on the 3′ end are incubated separately with the positive ligands, and their change in the intracellular cAMP level and/or the Ca2+ content caused by the specific binding is determined. Methods to perform such assays are conventional. Positive ligands which are not capable of causing such changes are suitable, for example, as antagonists for inhibition of maturation of spermatozoa and can be employed for the preparation of contraceptive agents.

Alternatively, assays can be carried out, in the presence or absence of ligands, for any of the ESRP-related physiological functions discussed hereinabove.

The use of such ligands for therapeutic purposes is particularly advantageous, since as a result of the tissue-specific expression of the receptor proteins according to the invention, this type of tissue is influenced selectively and no side effects, or only minor side effects, are therefore to be expected. From the foregoing description, one skilled in the art can easily ascertain the essential characteristics of this invention, and without departing from the spirit and scope thereof, can make changes and modifications of the invention to adapt it to various usage and conditions.

### Example 7

### Function and Phenotyping of HE6 Knock Out Animals

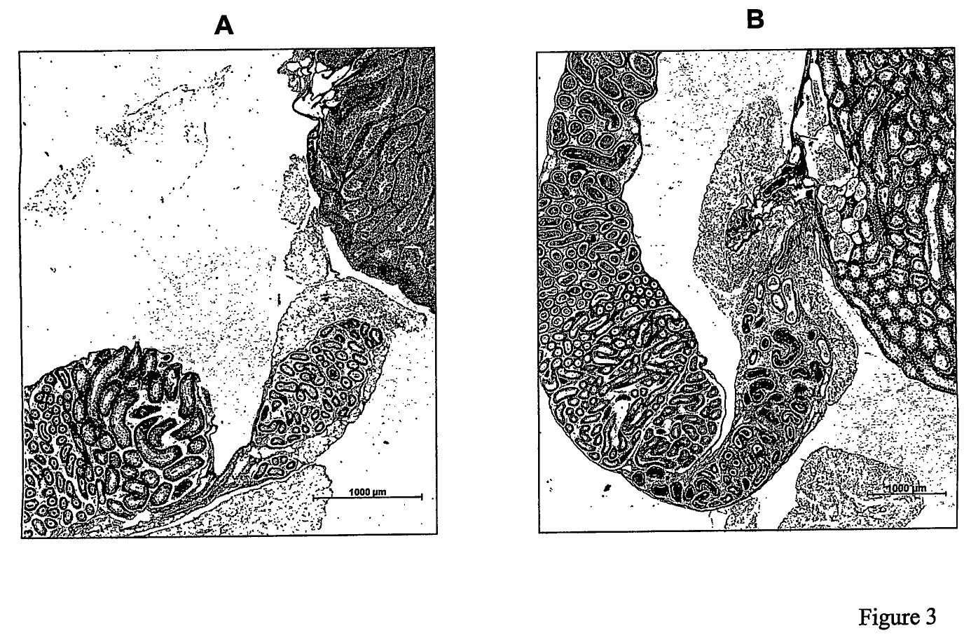

Through the generation of mouse harboring a targeted disruption of the murine HE6 gene the function of this receptor can be analysed: Testis, efferent and epididymal ducts from wild type and knock out mice (hemizygous male mice) are systematically compared using histological sections. Histological analysis from 6 weeks old and older mice revealed dilated ducts where HE6 is found to be highly expressed. In all examined mice an accumulation of spermatozoa within the distal part of the efferent ducts is observed (FIG. 3A). In mice of 8 weeks and older the accumulation of spermatozoa results in an obstruction so that the male mice become infertile.

The efferent ducts are mainly responsible for the absorption of testicular fluids transporting spermatozoa passively out of the testis. It is known from the ERα knock out mouse that reduced fluid resorbtion within the efferent ducts results in expanding rete testis caused by increased back pressure (Hess et al., Nature 390:509-512, (1997)). Histological examination of the rete testis from HE6 knock out mice shows drastically increased volumina of the rete testis (FIG. 3B). Therefore a defect or miss function in the fluid re-uptake caused directly or indirectly by the loss of HE6 appears to be probably.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of all applications, patents and publications, cited above and in the figures are hereby incorporated in their entirety by reference.

1. An isolated human epididymis-specific receptor protein-6 (HE6) polypeptide, comprising
i) the amino acid sequence encoded by SEQ ID NO: 1;
ii) the amino acid sequence encoded by SEQ ID NO: 2;
iii) the amino acid sequence encoded by SEQ ID NO: 3;
iv) the amino acid sequence encoded by SEQ ID NO: 4;
v) the amino acid sequence encoded by SEQ ID NO: 5;
vi) the amino acid sequence encoded by SEQ ID NO: 6; or
vii) the amino acid sequence encoded by SEQ ID NO: 7,
or comprising a functional variant or functional fragment of an amino acid sequence of i) through vii).

2. An isolated polypeptide that comprises an amino acid sequence which has a sequence identity of at least 65%, 70-75%, 80-85%, 90-95% or 97-99% to one or more of the amino acid sequences i) through vii) of claim 1.

3. An isolated polypeptide of claim 1, which further comprises a heterologous sequence.

4. An isolated polypeptide of claim 1, which comprises a polypeptide encoded by SEQ ID NO: 22 comprising the amino acid sequence as depicted in SEQ ID NO: 30.

5. An isolated polypeptide of claim 1, which comprises a polypeptide encoded by SEQ ID NO: 16-22, starting with AUG at nucleotide position 164-166 and terminating at stop codon at positions 3101-3103, 3143-3145, 3173-3175, 3167-3169, 3125-3127, or 3149-3151.

6. An isolated polypeptide encoded by SEQ ID NO: 16-22, starting at the codon at 2-4 and terminating at stop codon at positions 3101-3103, 3143-3145, 3173-3175, 3167-3169, 3125-3127, or 3149-3151.

7. An isolated polypeptide comprising, from N-terminus to C-terminus, the polypeptide sequence represented by amino acids 1 through 54 of SEQ ID NO: 22, covalently bound to the polypeptide X81892.

8. An isolated mouse epididymis-specific receptor protein-6 (ME6) polypeptide, comprising
i) the amino acid sequence encoded by SEQ ID NO: 9;
ii) the amino acid sequence encoded by SEQ ID NO: 10;
iii) the amino acid sequence encoded by SEQ ID NO: 11; or
iv) the amino acid sequence encoded by SEQ ID NO: 12;
or comprising a functional variant or functional fragment of an amino acid sequence of i) through iv).

9. An isolated polypeptide that comprises an amino acid sequence which has a sequence identity of at least 65%, 70-75%, 80-85%, 90-95% or 97-99% to one or more of the amino acid sequences i) through iv) of claim 8.

10. An isolated polypeptide consisting essentially of amino acids 1 to 1009 of SEQ ID NO: 31.

11. An isolated polypeptide of claim 8 which comprises amino acid sequences encoded by SEQ ID NO: 9, 10, 11, 12 or 13, and further comprises a heterologous sequence.

12. An isolated polypeptide of claim 8, which comprises a polypeptide encoded by SEQ ID NO: 23, 24, 25, or 26, starting with AUG at nucleotide position 72-74 and terminating at stop codons 3099-3101, 3051-3053, 3090-3092, and 3018-3020.

13. An isolated polypeptide of claim 8, which comprises the sequence beginning with amino acid 1 of the sequence represented by SEQ ID NO: 31.

14. An isolated rat epididymis-specific receptor protein-6 (RE6) polypeptide, comprising
i) the amino acid sequence encoded by SEQ ID NO: 13;
ii) the amino acid sequence encoded by SEQ ID NO: 14; or
iii) the amino acid sequence encoded by SEQ ID NO: 15
or comprising a functional variant or functional fragment of an amino acid sequence of i) through iii).

15. An isolated polypeptide that comprises an amino acid sequence which has a sequence identity of at least 65%, 70-75%, 80-85%, 90-95% or 97-99% to one or more of the amino acid sequences i) through iii) of claim 14.

16. An isolated polypeptide of claim 14 which comprises amino acid sequences encoded by SEQ ID NO: 13, 14, or 15, and further comprises a heterologous sequence.

17. An isolated polypeptide of claim 14, which comprises a polypeptide encoded by SEQ ID NO: 27, 28, or 29, starting with AUG at nucleotide position 60-62 and terminating at stop codons 3099-3101, and 3015-3017.

18. An isolated polypeptide of claim 14, which comprises the sequence beginning with amino acid 1 of the sequence represented by SEQ ID NO: 32.

19. An isolated polynucleotide which encodes an HE6-polypeptide, or which encodes a functional variant or a functional fragment thereof, of claim 1.

20. An isolated polynucleotide which comprises the nucleotide sequence of
i) SEQ ID NO: 1,
ii) SEQ ID NO: 2,
iii) SEQ ID NO: 3,
iv) SEQ ID NO: 4,
v) SEQ ID NO: 5,
vi) SEQ ID NO: 6, or
vii) SEQ ID NO: 7,
or a functional fragment or variant thereof, provided that said fragment comprises either SQ ID NO: 1, 2, 3, 4, 5, 6, or 7.

21. An isolated polynucleotide which comprises the nucleotide sequence of
i) SEQ ID NO: 9
ii) SEQ ID NO: 10,
iii) SEQ ID NO: 11, or
iv) SEQ ID NO: 12
or a functional fragment or variant thereof, provided that said fragment comprises either SQ ID NO: 9, 10, 11, or 12.

22. An isolated polynucleotide which comprises the nucleotide sequence of
i) SEQ ID NO: 13,
ii) SEQ ID NO: 14, or
iii) SEQ ID NO: 15
or a functional fragment or variant thereof, provided that said fragment comprises either SQ ID NO: 13, 14, or 15.

23. Isolated polynucleotides of claim 20 which further comprise heterologous sequences.

24. An isolated polynucleotide comprising from the 5′ to the 3′ terminus, an oligonucleotide consisting essentially of nucleotides 1-91 of SEQ ID NO: 16 covalently bound, in phase, to the polynucleotide X81892.

25. An isolated polynucleotide of claim 20, which comprises SEQ ID NOS:16, 17, 18, 19, 20, 21 or 22.

26. An isolated polynucleotide of claim 21, which comprises SEQ ID NOS: 23, 24, 25, or 26.

27. An isolated polynucleotide of claim 22, which comprises SEQ ID NOS:27, 28, or 29.

28. An isolated polynucleotide which comprises a nucleotide sequence that codes without interruption for the polypeptide of SEQ ID NO: 30, or which comprises a nucleotide sequence that codes without interruption for a fragment or variant of the polypeptide of SEQ ID NO: 30, or a complement thereof.

29. Recombinant constructs comprising the polynucleotides of claim 20 operatively linked to a regulatory sequence.

30. A cell comprising a polynucleotide of claim 20 which expresses an HE-6 polypeptide.

31. A cell comprising a polynucleotide of claim 21 which expresses an ME-6 polypeptide.

32. A cell comprising a polynucleotide of claim 22 which expresses an RE-6 polypeptide.

33. A method of making epididymis-specific receptor-6 polypeptides, or functional fragments or variants thereof, comprising incubating a cell of claim 35 under conditions which allow expression of said polypeptides, fragments or variants, and recovering the polypeptides, fragments or variants.

34. An antibody or antigen-specific fragment specific for a polypeptide comprising amino acid sequences of claim 1.

35. An antibody according to claim 34, wherein said antibody is a monoclonal antibody.

36. A pharmaceutical composition comprising an antagonist or inhibitor of a polypeptide comprising amino acid sequences encoded by SEQ ID NO: 1, 2, 3, 4, 5, 6, 7, 8, 16, 17, 18, 19, 20, 21, or 22 and a pharmaceutically acceptable carrier.

37. A pharmaceutical composition comprising an antisense nucleotide which can bind with any of the nucleotide sequences shown in SEQ ID NO: 1, 2, 3, 4, 5, 6, 7, 8, 16, 17, 18, 19, 20, 21, or 22 and a pharmaceutically acceptable carrier.

38. A pharmaceutical composition comprising an antibody of claim 34 and a pharmaceutically acceptable carrier.

39. A pharmaceutical composition for treating a male reproductive disorder comprising administering an effective amount of a polypeptide comprising amino acid sequences encoded by SEQ ID NO: 1, 2, 3, 4, 5, 6, 7, 8, 16, 17, 18, 19, 20, 21, or 22.

40. A pharmaceutical composition for diagnosing a male reproductive disorder comprising administering an effective amount of a polypeptide comprising amino acid sequences encoded by SEQ ID NO: 1, 2, 3, 4, 5, 6, 7, 12, 13, 14, 15, 27, 28, or 29.

41. A method of isolating an agent which modulates expression activity of an epididymis-specific receptor of claim 1, or of a polynucleotide which encodes it, comprising incubating said epididymis-specific receptor or polynucleotide with a putative agent, and measuring the amount of activity of said receptor or polynucleotide.

42. The method of claim 41 wherein said agent is an antisense oligonucleotide of this invention.

43. The method of claim 41 wherein said agent is a ligand of said receptor.

44. A method for isolating a ligand specific for an epididymis-specific receptor of claim 1, comprising contacting the epididymis-specific receptor with a substance suspected to be a ligand of said receptor and detecting binding of said receptor to said ligand.

45. The method of claim 41, wherein the said agent or ligand is an agonist of the epididymis-specific receptor.

46. The method of claim 41, wherein the said agent or ligand is an antagonist of the epididymis-specific receptor.

47. The method of claim 41, wherein the said agent or ligand is an antibody or polypeptide.

48. The method of claim 41, wherein the said agent or ligand is a small molecule which binds to the epididymis-specific receptor.

49. A method for diagnosing infertility in a male mammal, which is associated with under-expression or over-expression of a polynucleotide comprising a sequence of SEQ ID NO: 1, 2, 3, 4, 5, 6, 7, 8, 16, 17, 18, 19, 20, 21, or 22, comprising contacting a tissue, cell, or polynucleotide from said male with a probe that is specific for said SEQ ID and determining the amount of nucleic acid that hybridizes to the probe wherein said cell or tissue is from a biopsy sample or thin section from the epididymis of said male mammal.

50. A method for diagnosing infertility in a male mammal comprising measuring antibodies from said male specific for a polypeptide comprising a sequence of SEQ ID NO: 1, 2, 3, 4, 5, 6, 7, 8, 16, 17, 18, 19, 20, 21 or 22.

51. A method for treating infertility in a male mammal comprising administering to said mammal an agonist or antagonist of an epididymis-specific receptor comprising administering an effective amount of a polypeptide comprising amino acid sequence encoded by SEQ ID NO: 1, 2, 3, 4, 5, 6, 7, 8, 16, 17, 18, 19, 20, 21 or 22.

52. A method for contraception in a male mammal comprising administering to said mammal an antagonist of an epididymis-specific receptor comprising a polypeptide encoded by SEQ ID NO: 1, 2, 3, 4, 5, 6, 7, 8, 16, 17, 18, 19, 20, 21 or 22.

53. A recombinant construct comprising the polynucleotide of SEQ ID NO:33 operatively linked to a regulatory sequence.

54. An antibody or an antigen-specific fragment specific for human HE6 polypeptide comprising amino acid sequences encoded by SEQ ID NO:33.

55. A pharmaceutical composition comprising an antagonist or inhibitor of a polypeptide comprising amino acid sequences encoded by SEQ ID NOS: 34 or 35.

56. A pharmaceutical composition for treating a male reproductive disorder comprising administering an effective amount of a polypeptide comprising an amino acid sequence encoded by SEQ ID NO:33.

57. A composition for diagnosing a male reproductive disorder comprising administering an effective amount of a polypeptide comprising amino acid sequences encoded by SEQ ID NO:33.

58. Isolated proteins encoded by DNA of claim 25, comprising the amino acid sequences of SEQ ID NO 36-60.
